(19) United States
(12) Patent Application Publication
(10) Pub. No.: US 2026/0152498 A1
(43) Pub. Date: Jun. 4, 2026

(54) 4,5,6,7-TETRAHYDRO-1H-PYRAZOLO[4,3-c]PYRIDINE COMPOUNDS AND DERIVATIVES AS USP1 INHIBITORS

(71) Applicant: EXELIXIS, INC., Alameda, CA (US)

(72) Inventors: Chandrasekhar ABBINENI, Hyderabad (IN); Susanta SAMAJDAR, Bangalore (IN); Madhu AELURI, Bangalore (IN); Scott SIMONOVICH, Alameda, CA (US)

(73) Assignee: EXELIXIS, INC., Alameda, CA (US)

(21) Appl. No.: 19/122,606

(22) PCT Filed: Oct. 20, 2023
(86) PCT No.: PCT/US2023/077411

§ 371 Date: Apr. 18, 2025

(30) Foreign Application Priority Data
Oct. 21, 2022 (IN) 202241060218

(51) Int. Cl. C07D 471/04; A61K 31/437; A61K 31/444; A61K 31/506; A61P 35/00

(52) U.S. Cl. CPC C07D 471/04; A61K 31/437; A61K 31/444; A61K 31/506; A61P 35/00

(57) ABSTRACT

The present disclosure provides 4,5,6,7-tetrahydro-1H-pyrazolo[4,3-c]pyridine compounds of formula (I), which are therapeutically useful as USP1 inhibitors. These compounds are useful in the treatment and/or prevention of diseases and/or disorders responsive to the inhibition of USP1 proteins and USP1 activity. Compounds of the present disclosure are especially useful for treating cancer. The present disclosure also provides processes for preparation of the compounds and pharmaceutical formulations comprising at least one of the compounds of formula (I) or a pharmaceutically acceptable salt or a stereoisomer thereof.

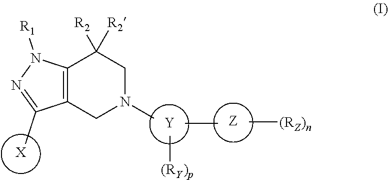

### CROSS-REFERENCES TO RELATED APPLICATIONS

The present application claims priority to Indian Pat. Appl. No. 202241060218, filed on Oct. 21, 2022, which application is incorporated herein by reference in its entirety.

### TECHNICAL FIELD

The present application is directed to 4,5,6,7-tetrahydro-1H-pyrazolo[4,3-c]pyridine compounds of formula (I) as USP1 inhibitors, useful for the treatment of cancer and inflammatory diseases or disorders. The disclosure also provides pharmaceutically acceptable compositions comprising compounds of the present application and methods of using said compounds and compositions in the treatment of diseases associated with USP1.

### BACKGROUND

Deubiquitinases (DUBs) are a class of enzymes that act on ubiquitinated substrates to catalyze the removal of ubiquitin moieties. The human genome contains approximately 100 genes that encode DUBs. Human DUBs are classified into five different families (Nijman, S. M. et al. (2005) Cell 123, 773-86, Nalepa, G. et al. (2006) Nat Rev Drug Discov 5, 596-613). USP1 (ubiquitin specific protease 1) belongs to the USP subfamily of DUBs. The USP1 gene, encodes a 785 amino acid protein that constitutes a conserved USP domain amino-terminal Cys box motif and a carboxy-terminal His box motif (Nijman, S. M. et al. (2005) Mol Cell 17, 331-9). The interaction of USP1 with UAF1, a WD40 repeat-containing protein, leads to formation of an activated USP1/UAF1 complex, which is required for the deubiquitinase activity of USP1 (Cohn, M. A. et al. (2007) Mol Cell 28, 786-97, Cohn, M. A. et al. (2009) J Biol Chem 284, 5343-51). USP1 gene transcription is regulated in a cell cycle-dependent manner. mRNA levels of USP-1 remain low during Gi phase and reach a peak during S phase (Nijman, S. M. et al. (2005) Mol Cell 17, 331-9). The expression of USP1 is also regulated at the protein level by proteasomal degradation (Cataldo F, Mol Cell Biol (2013). 33(12):2485-2496).

USP1 is a nuclear protein and localizes to chromatin where it is specifically associated with Fanconi anemia protein FANCD2. USP1 acts as a regulator and governs several important steps in the DNA damage response pathway, which include the Fanconi anemia (FA) pathway and the process of translesion synthesis (TLS). USP1 deubiquitinates monoubiquitinated FANCD2, which plays an important role in DNA damage repair (Nijman, S. M. et al. (2005) Mol Cell 17, 331-9, Guervilly, J. H. et al. (2011) Hum Mol Genet). While DNA-dependent mono-ubiquitination of FANCD2 facilitates DNA repair, it is deubiquitinated by USP1 to block the DNA-repairing response. USP1 is also critical for the deubiquitination of monoubiquitinated PCNA and thus negatively regulates PCNA-mediated TLS during DNA repair (Huang T T. et al. Nat Cell Biol (2006), 8(4):339-347). The expression of USP1 is significantly increased in several cancers (Das D S. et al. C/in Cancer Res. (2017) 23:4280-9. Xin Xu, et al. Front Oncol. (2019) 9:1406). Inhibition of USP1 inhibited DNA repair and induced cell death in multiple myeloma cells (Das D S. Et al. Clin Cancer Res. (2017) 23:4280-9). Lung cancer cells are sensitized to cisplatin upon inhibition of USP1 activity (Chen J, et al. Chem Biol. (2011) 18:1390-400). The results from these studies indicate that USP1 is a promising anti-cancer target.

Only a few USP1 inhibitors are reported in the literature including those described in WO2007149484, WO2011137320, WO2014105952, WO2017087837, WO2019089216, WO2020132269, WO2020139988, WO2021163530, WO2022174184 and WO2021247606.

Inhibition of USP1 with small molecule inhibitors therefore has the potential to be a treatment for cancers and other disorders. It is, therefore, an object of this disclosure to provide compounds useful in the treatment of such diseases and/or disorders responsive to the inhibition of USP1 proteins and USP1 activity.

### SUMMARY

Provided herein are 4,5,6,7-tetrahydro-1H-pyrazolo[4,3-c]pyridine compounds of formula (I) and pharmaceutical compositions thereof, which are capable of inhibiting USP1.

In one aspect, the present disclosure comprises compounds of formula (I):

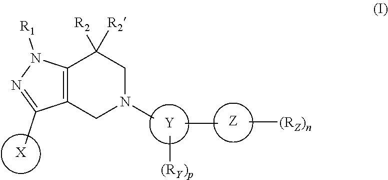

or a pharmaceutically acceptable salt thereof or a stereoisomer thereof, wherein:
ring X is

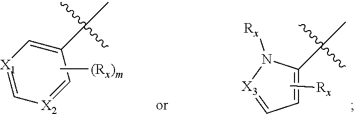

ring Y is

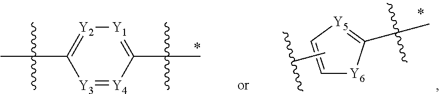

wherein the asterisk marks the point of attachment to ring Z;
ring Z is heterocyclyl;
each X₁, X₂ and X₃ independently is N or C;
each Y₁ to Y₄ independently is N or C; wherein 0-2 of Y₁ to Y₄ are N;
each Y₅ and Y₆ is independently N, C or S;
R_{X} at each occurrence independently is alkyl, alkoxy or cycloalkyl;
R_{Y} is halo, alkyl, alkoxy or alkylamino;
R_{Z} at each occurrence is independently selected from hydrogen, halo, alkyl, haloalkyl, alkoxy, cycloalkyl, alkylaminoalkyl, alkoxyalkyl and unsubstituted or alkyl-substituted heterocycloalkyl;
R₁ is selected from hydrogen, alkyl, cycloalkyl, alkylaminoalkyl, unsubstituted or alkyl-substituted heterocycloalkyl, unsubstituted or substituted arylalkyl, and unsubstituted or substituted heteroarylalkyl, wherein the arylalkyl and heteroarylalkyl substituents are selected from halo, alkyl and alkoxy;
R₂ and R_{2′} each independently is hydrogen or alkyl;
m is 1, 2 or 3;
n is 1, 2 or 3; and
p is 0 or 1.

Also provided are pharmaceutical compositions comprising a compound of formula (I) or a pharmaceutically acceptable salt thereof and at least one pharmaceutically acceptable excipient (such as a pharmaceutically acceptable carrier or diluent).

Also provided are processes for the preparation of compounds of formula (I).

Compounds of formula (I) are useful for the inhibition of USP1, and therapeutic use of such compounds is also provided.

### DETAILED DESCRIPTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated to facilitate the understanding of the present disclosure.

As used herein, unless otherwise defined the term “alkyl,” alone or in combination with other term(s), means saturated aliphatic hydrocarbon chains, including C₁-C₁₀ straight or C₁-C₁₀ branched alkyl chains, more preferably, C₁-C₆ straight or branched alkyl chains. Examples of “alkyl” include but are not limited to methyl, ethyl, propyl, isopropyl, butyl, isobutyl, sec-butyl, tert-butyl, isopentyl or neopentyl and the like.

As used herein, the term “alkenyl,” alone or in combination with other term(s), refers to an alkyl group as described above having at least one carbon-carbon double bond. The term “alkynyl,” alone or in combination with other term(s), refers to an alkyl group as described above having at least one carbon-carbon triple bond.

As used herein, the term “halo” or “halogen,” alone or in combination with other term(s), means fluorine, chlorine, bromine or iodine.

As used herein, the term “haloalkyl,” alone or in combination with other term(s), means alkyl substituted with one or more halogen atoms, wherein the alkyl groups are as defined above. The term “halo” is used herein interchangeably with the term “halogen” means F, Cl, Br or I. Examples of “haloalkyl” include but are not limited to fluoromethyl, difluoromethyl, chloromethyl, trifluoromethyl. 2,2,2-trifluoroethyl and the like.

As used herein, the term “alkoxy,” alone or in combination with other term(s), refers to the group alkyl-O— or —O-alkyl, where alkyl groups are as defined above. Exemplary C₁-C₁₀ alkoxy- groups include but are not limited to methoxy, ethoxy, n-propoxy, n-butoxy, t-butoxy and the like. An alkoxy group can be unsubstituted or substituted with one or more suitable groups.

As used herein, the term “alkoxyalkyl,” alone or in combination with other term(s), means the “alkoxy” as defined above linked to the rest of the molecule via an alkyl moiety. Examples of “alkoxyalkyl” include but not limited to —CH₂—OCH₃, —C₂H₅—OCH₃, —CH₂—OCH₂CH₃, —CH₂—OC₃H₇, —C₂H₅—OCH₂CH₃ and the like.

The term “amino” or “amine,” alone or in combination with other term(s), refers to a primary amine (—NH₂), secondary amine

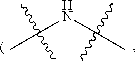 [SMILES: *N*]

wherein ‘N’ is substituted with two substituents other than hydrogen) or tertiary amine

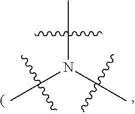 [SMILES: *N(*)*]

wherein ‘N’ is substituted with three substituents other than hydrogen) group.

As used herein, the term “alkylamino,” alone or in combination with other term(s), means an amino group as defined above, substituted with one or more “alkyl” group, wherein the alkyl group and amino group is as defined above. Examples of “alkylamino” groups include but are not limited to —NHCH₃, —NHCH₂CH₃, —N(CH₃)₂, —N(CH₃)(CH₂CH₃) and the like.

As used herein, the term “alkylaminoalkyl,” alone or in combination with other term(s), means the “alkylamino” as defined above linked to the rest of the molecule via an alkyl moiety. Examples of “alkylaminoalkyl” include but not limited to —CH₂—NHCH₃, —C₂H₅—NHCH₃, —CH₂—NHCH₂CH₃, —CH₂—N(CH₃)₂, —CH₂—N(CH₃)(CH₂CH₃) and the like.

As used herein the term “cycloalkyl,” alone or in combination with other term(s), means —C₃-C₁₀ saturated cyclic hydrocarbon ring. A cycloalkyl may be a single ring, which typically contains from 3 to 7 carbon ring atoms. Examples of single-ring cycloalkyls include but are not limited to cyclopropyl, cyclobutyl, cyclopentyl, cyclohexyl, cycloheptyl and the like. A cycloalkyl may alternatively be polycyclic or contain more than one ring. Examples of polycyclic cycloalkyls include bridged, fused and spirocyclic carbocyclyls and the like.

As used herein, the term “aryl,” alone or in combination with other term(s), is unsubstituted or substituted monocyclic, bicyclic or polycyclic aromatic hydrocarbon ring system of about 6 to 14 carbon atoms. Examples of a C₆-C₁₄ aryl group include, but are not limited to phenyl, naphthyl, anthryl, biphenylenyl and acenaphthyl. An aryl group may be unsubstituted or substituted with one or more suitable groups.

As used herein, the term “arylalkyl,” alone or in combination with other term(s), means the aryl as defined above is linked to the rest of the molecule via an alkyl moiety. Examples for arylalkyl include but are not limited to (phenyl)alkyl-, (naphthyl)alkyl-, (anthryl)alkyl- and the like.

As used herein, the term “carbocyclyl,” alone or in combination with other term(s), includes both “cycloalkyl” and “aryl” groups which are as defined above. Examples of “carbocyclyl” include, but are not limited to cyclopropyl, cyclobutyl, cyclopentyl, cyclohexyl, phenyl and naphthyl.

The term “heterocycloalkyl,” alone or in combination with other term(s), refers to a non-aromatic, saturated or partially saturated monocyclic or polycyclic ring system of 3 to 15 members having at least one heteroatom or heterogroup selected from O, N, S, S(O), S(O)₂, and NH with the remaining ring atoms being independently selected from the group consisting of carbon, oxygen, nitrogen and sulfur. A monocyclic heterocycloalkyl may typically contain 4 to 7 ring atoms. Examples of “heterocycloalkyl” include, but are not limited to azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, oxapiperazinyl, oxapiperidinyl, tetrahydrothiophenyl, dihydropyranyl, indolinyl, azepanyl and N-oxides thereof. Attachment of a heterocycloalkyl substituent can occur via either a carbon atom or a heteroatom. A heterocycloalkyl group can be unsubstituted or substituted with one or more suitable groups.

As used herein, the term “heteroaryl,” alone or in combination with other term(s), means a completely unsaturated ring system containing a total of 5 to 14 ring atoms. At least one of the ring atoms is a heteroatom (oxygen, nitrogen, or sulfur), with the remaining ring atoms/groups being independently selected from the group consisting of carbon, oxygen, nitrogen and sulfur. A heteroaryl may be a single-ring (monocyclic) or polycyclic ring system. Examples of “heteroaryl” include but are not limited to pyridyl, indolyl, benzimidazolyl, benzothiazolyl, pyrrolyl, pyrazolyl, imidazolyl, pyrimidinyl, pyrazinyl, pyridazinyl and the like.

As used herein, the term “heteroarylalkyl,” alone or in combination with other term(s), means the heteroaryl as defined above is linked to the rest of the molecule via an alkyl moiety. Examples of heteroarylalkyl include but not limited to (pyridine)alkyl-, (indole)alkyl-, (benzimidazole)alkyl-, (pyrrole)alkyl-, (pyrazole)alkyl-. (imidazole)alkyl-, (pyrimidine)alkyl-, (pyrazine)alkyl-, (pyridazine)alkyl- and the like.

As used herein, the term “heterocyclyl,” alone or in combination with other term(s), includes both “heterocycloalkyl” and “heteroaryl” groups which are as defined above. Examples of “Heterocyclyl” include, but are not limited to azetidinyl, pyrrolidinyl, piperidinyl, pyridyl, indolyl, benzimidazolyl, benzothiazolyl and the like.

The term “heteroatom” as used herein designates a sulfur, nitrogen or oxygen atom.

As used in the above definitions, the term “optionally substituted” or “substituted” or “optionally substituted with suitable groups” refers to replacement of one or more hydrogen radicals in a given structure with a radical of a specified substituent including, but not limited to: halo, alkyl, alkenyl, alkynyl, aryl, heterocycloalkyl, heteroaryl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl (e.g., trifluoromethyl), amino, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, arylalkoxycarbonyl, carboxylic acid, sulfonic acid, sulfonyl, and phosphonic acid. It is understood that any substituent may be further substituted.

As used herein, the term “compound(s)” comprises the compounds disclosed in the present disclosure.

As used herein, the terms “comprise” and “comprising” are generally used in the sense of “include” and “including,” that is to say permitting the presence of one or more additional features or components. The term “including” as well as other forms thereof, such as “include”, “includes” and “included” is not limiting.

As used herein, the term “or” means “and/or” unless stated otherwise.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the phrase “pharmaceutically acceptable excipient” refers to pharmaceutically substances such as a liquid or solid filler, diluent, solvent, or encapsulating material. By “pharmaceutically acceptable” it is meant that the substance is compatible with the other ingredients of a composition or formulation and not deleterious to the recipient thereof. Excipients are generally safe, non-toxic and neither biologically nor otherwise undesirable, including those which are acceptable for human pharmaceutical use as well as veterinary use. See, e.g., Remington: The Science and Practice of Pharmacy, 23rd Ed. (Academic Press, 2020); Handbook of Pharmaceutical Excipients, 9th ed., Sheskey et al, Eds. (Pharmaceutical Press; 2020); Handbook of Pharmaceutical Additives, 3rd ed.; Ash and Ash Eds.: Gower Publishing Company: 2007; Pharmaceutical Preformulation and Formulation, 2nd ed.; Gibson Ed.; CRC Press LLC: Boca Raton, Fla., 2009.

As used herein, “pharmaceutically acceptable salt(s)” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include non-toxic salts of the parent compound formed, for example, from non-toxic inorganic or organic acids. The pharmaceutically acceptable salts of the present disclosure can be synthesized from the parent compound which contains a basic or acidic moiety by various chemical methods. Generally, such salts can be prepared by reacting the free acid or base forms of these compounds with a stoichiometric amount of the appropriate base or acid in water or in an organic solvent, or in a mixture of the two.

As used herein, the terms “treat”, “treating” and “treatment” refer to a method of alleviating or abrogating a disease and/or its attendant symptoms.

As used herein, the terms “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “therapeutically effective amount” refers to the amount of the compound being administered sufficient to prevent development of, or to partially or completely alleviate one or more of the symptoms of, the condition or disorder being treated.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

As used herein, the phrase “pharmaceutically acceptable excipient” refers to a pharmaceutically acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In some embodiments, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al, Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, “pharmaceutically acceptable salt(s)” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols or acetonitrile (ACN) are preferred.

The term “stereoisomers” refers to any enantiomers, diastereoisomers, or geometrical isomers of the compounds of Formula (I), (IA), (IB), (IC), (ID), (IE), (IF), (IG), (IH), (U), and (IK), and Formula (II), (III), (IV), (V), (VI), and (VII) wherever they are chiral or when they bear one or more double bonds. When the compounds of the formula (I), (IA), (IB), (IC), (ID), (IE), (IF), (IG), (IH), (IJ), and (IK), and Formula (II), (III), (IV), (V), (VI), and (VII) are chiral, they can exist in racemic or in optically active form. It should be understood that the disclosure encompasses all stereochemical isomeric forms, including diastereomeric, enantiomeric and epimeric forms, as well as d-isomers and l-isomers and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation via recrystallization, chromatographic techniques (e.g., direct separation of enantiomers on chiral chromatographic columns), or any other appropriate method known in the art. Starting compounds of particular stereochemistry are either commercially available or can be made and resolved by techniques known in the art. Additionally, the compounds of the present disclosure may exist as geometric isomers. The present disclosure includes all cis and trans, syn and anti, (R) and (S), entgegen (E) and zusammen (Z) isomers as well as mixtures thereof.

The present disclosure provides 4,5,6,7-tetrahydro-1H-pyrazolo[4,3-c]pyridine compounds of formula (I), which are useful for the inhibition of USP1.

The present disclosure further provides pharmaceutical compositions comprising the said compounds of formula (I), and their derivatives as therapeutic agents.

It will be apparent to those skilled in the art that various modifications and variations can be made to the compounds, compositions, and methods described herein without departing from the scope or spirit of various embodiments disclosed herein. For instance, features illustrated or described as part of one embodiment can be applied to another embodiment to yield a still further embodiment. Thus, it is intended that the present disclosure includes such modifications and variations and their equivalents. It is to be understood by one of ordinary skill in the art that the present discussion is a description of exemplary embodiments and is not to be construed as limiting the broader aspects of the present disclosure.

The embodiments below are illustrative of the present disclosure and are not intended to limit the claims to the specific embodiments exemplified.

In some embodiments, 4,5,6,7-tetrahydro-1H-pyrazolo[4,3-c]pyridine compounds of formula (I) are provided:

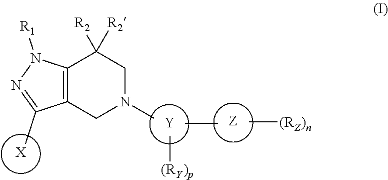

as well as pharmaceutically acceptable salts thereof or stereoisomers thereof, wherein:
ring X is

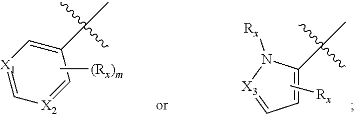

ring Y is

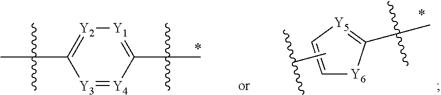

wherein the asterisk marks the point of attachment to ring Z;
ring Z is heterocyclyl;
each X₁, X₂ and X₃ independently is N or C;
each Y₁ to Y₄ independently is N or C; wherein 0-2 of Y₁ to Y₄ are N;
each Y₅ and Y₆ is independently N, C or S;
R_{X} at each occurrence independently is alkyl, alkoxy or cycloalkyl;
R_{Y} is halo, alkyl, alkoxy or alkylamino;
R_{Z} at each occurrence is independently selected from hydrogen, halo, alkyl, haloalkyl, alkoxy, cycloalkyl, alkylaminoalkyl, alkoxyalkyl, and unsubstituted or alkyl-substituted heterocycloalkyl;
R₁ is selected from hydrogen, alkyl, cycloalkyl, alkylaminoalkyl, unsubstituted or alkyl-substituted heterocycloalkyl, unsubstituted or substituted arylalkyl, and unsubstituted or substituted heteroarylalkyl, wherein the arylalkyl and heteroarylalkyl substituents are selected from halo, alkyl and alkoxy;
R₂ and R_{2′} each independently is hydrogen or alkyl;
m is 1, 2 or 3;
n is 1, 2 or 3; and
p is 0 or 1.

In some embodiments, compounds of formula (IA),

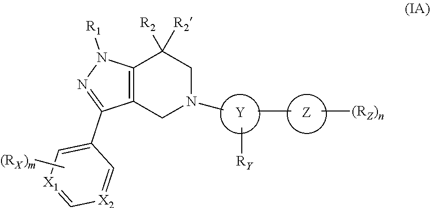

or pharmaceutically acceptable salts thereof or stereoisomers thereof, are provided.

In some embodiments, compounds of formula (IB),

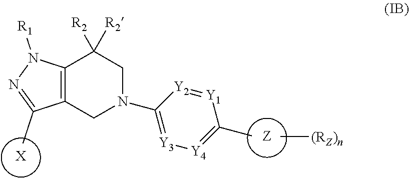

or pharmaceutically acceptable salts thereof or stereoisomers thereof, are provided.

In some embodiments, compounds of formula (IC),

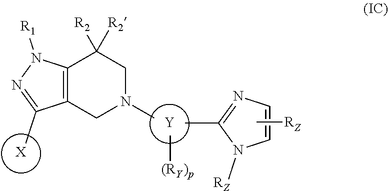

or pharmaceutically acceptable salts thereof or stereoisomers thereof, are provided.

In some embodiments, compounds of formula (ID),

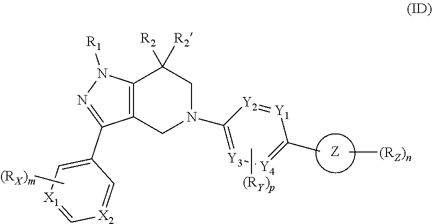

or pharmaceutically acceptable salts thereof or stereoisomers thereof, are provided.

In some embodiments, compounds of formula (IE),

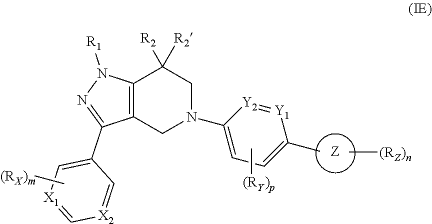

or pharmaceutically acceptable salts thereof or stereoisomers thereof, are provided.

In some embodiments, compounds of formula (IF),

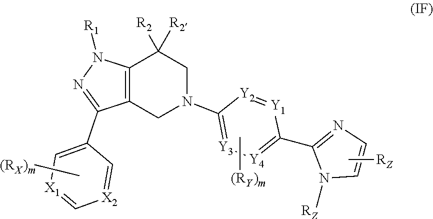

or pharmaceutically acceptable salts thereof or stereoisomers thereof, are provided.

In some embodiments, compounds of formula (IG),

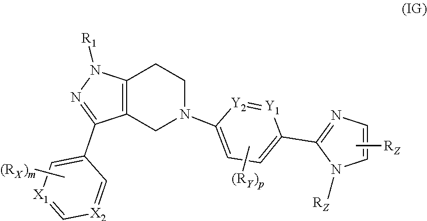

or pharmaceutically acceptable salts thereof or stereoisomers thereof, are provided.

In some embodiments, compounds of formula (IH),

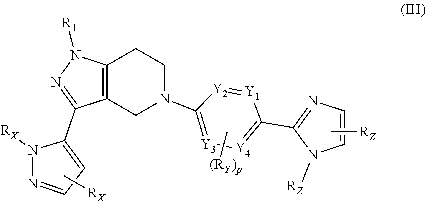

or pharmaceutically acceptable salts thereof or stereoisomers thereof, are provided.

In some embodiments, compounds of formula (IJ),

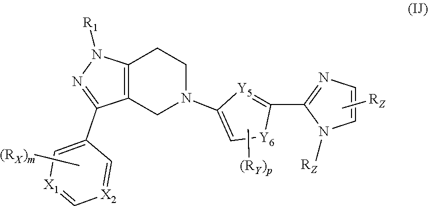

or pharmaceutically acceptable salts thereof or stereoisomers thereof, are provided.

In some embodiments of the compound of formula (IJ),
each X₁ and X₂ independently is N or C;
each Y₅ and Y₆ independently is N, C or S;
R_{X} at each occurrence independently is alkyl, alkoxy or cycloalkyl;
R_{Y} is halo, alkyl, alkoxy or alkylamino;
R_{Z} at each occurrence is independently selected from hydrogen, halo, alkyl, haloalkyl, alkoxy, cycloalkyl, alkylaminoalkyl, alkoxyalkyl, and unsubstituted or alkyl-substituted heterocycloalkyl;
R₁ is selected from hydrogen, alkyl, cycloalkyl, alkylaminoalkyl, unsubstituted or alkyl-substituted heterocycloalkyl, unsubstituted or substituted arylalkyl, and unsubstituted or substituted heteroarylalkyl, wherein the arylalkyl and heteroarylalkyl substituents are selected from halo, alkyl and alkoxy;
m is 1, 2 or 3; and
p is 0 or 1.

In some embodiments, the compound of formula (IJ) is selected from the group consisting of:
3-(4-cyclopropyl-6-methoxypyrimidin-5-yl)-1-methyl-5-(5-(1-methyl-4-(trifluoromethyl)-1H-imidazol-2-yl)thiophen-3-yl)-4,5,6,7-tetrahydro-1H-pyrazolo[4,3-c]pyridine; and
4-(3-(4-cyclopropyl-6-methoxypyrimidin-5-yl)-1-methyl-1,4,6,7-tetrahydro-5H-pyrazolo[4,3-c]pyridin-5-yl)-2-(1-methyl-4-(trifluoromethyl)-1H-imidazol-2-yl)thiazole;
or a pharmaceutically acceptable salt or a stereoisomer thereof.

In some embodiments, compounds of formula (IK),

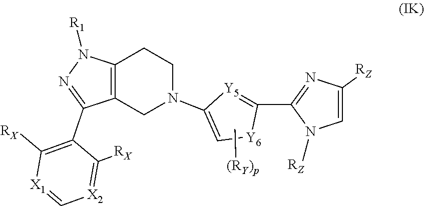

or pharmaceutically acceptable salts thereof or stereoisomers thereof, are provided.

In some embodiments, 4,5,6,7-tetrahydro-IH-pyrazolo[4,3-c]pyridine compounds of formula (I),

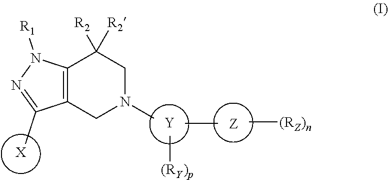

or pharmaceutically acceptable salts thereof or stereoisomers thereof, are provided, wherein:
ring X is

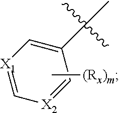

ring Y is

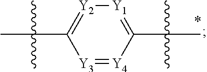

wherein the asterisk marks the point of attachment to ring Z;
ring Z is heterocyclyl;
each X₁ and X₂ independently is N or C;
each Y₁ to Y₄ independently is N or C; wherein 0-2 of Y₁ to Y₄ are N; each Y₅ and Y₆ is independently N, C or S;
R_{X} at each occurrence independently is alkyl, alkoxy or cycloalkyl;
R_{Y} is halo, alkyl, alkoxy or alkylamino;
R_{Z} at each occurrence is independently selected from hydrogen, halo, alkyl, haloalkyl, alkoxy, cycloalkyl, alkylaminoalkyl and, unsubstituted or alkyl-substituted heterocycloalkyl;
R₁ is selected from hydrogen, alkyl, cycloalkyl, alkylaminoalkyl unsubstituted or alkyl substituted heterocycloalkyl, unsubstituted or substituted arylalkyl, and unsubstituted or substituted heteroarylalkyl, wherein the arylalkyl and heteroarylalkyl substituents are selected from halo, alkyl and alkoxy;
R₂ and R_{2′} each independently is hydrogen or alkyl;
m is 1, 2 or 3; and
n is 1, 2 or 3; and
p is 0 or 1.

In some embodiments, the ring X is

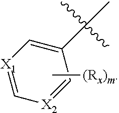

In some embodiments, the ring

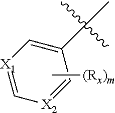

is,

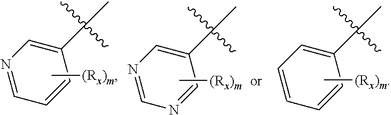

In some embodiments, ring X is

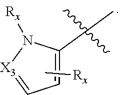

In some embodiments, the ring Y is

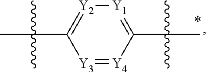

wherein the asterisk marks the point of attachment to ring Z.

In some embodiments, the ring

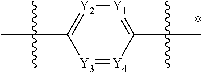

is

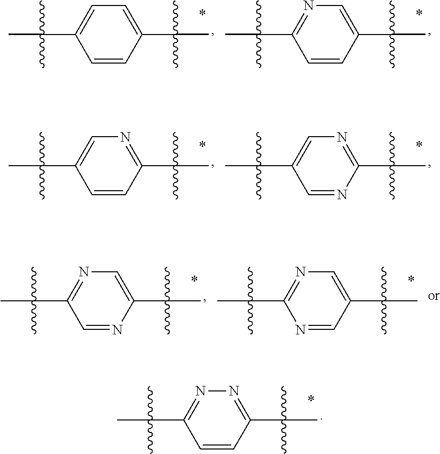

In some embodiments, the ring

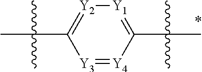

is

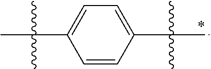 [SMILES: *c1ccc(*)cc1]

In some embodiments, the ring

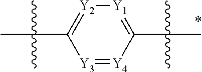

is

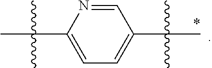 [SMILES: *c1ccc(*)nc1]

In some embodiments, the ring

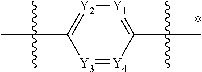

is.

In some embodiments, the ring Y is

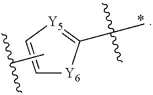

In some embodiments, the ring

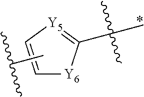

is

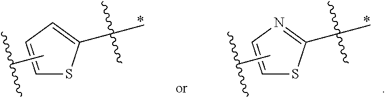

In some embodiments, the ring Z is 5 to 6 membered heteroaryl or 3 to 12 membered heterocycloalkyl.

In some embodiments, the ring Z is 5 to 6 membered heteroaryl. In some embodiments, the 5 to 6 membered heteroaryl is

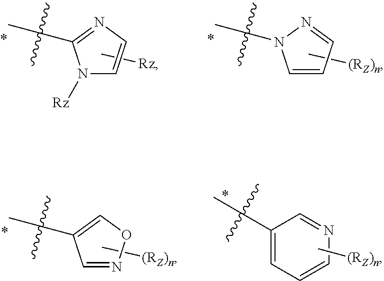

wherein the asterisk marks the point of attachment to ring Y.

In some embodiments, the ring Z is a heterocycloalkyl ring. In some embodiments, the ring Z is a partially saturated bicyclic heterocycloalkyl ring. In some embodiments, the heterocycloalkyl is

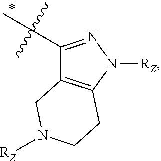

wherein the asterisk marks the point of attachment to ring Y.

In some embodiments, when the ring Z is a partially saturated bicyclic heterocycloalkyl ring, the substituent Rz can be present on any of the ring atoms.

In some embodiments, the substituent R_{Z} can be present on any of the ring atoms.

In some embodiments, the substituent R_{X} can be present on any of the ring atoms.

In some embodiments, the substituent R_{Y} can be present on any of the ring atoms.

In some embodiments, the substituents R_{X}, R_{Y} and R_{Z} can be present on either carbon or any heteroatom of the ring atom.

In some embodiments, R₁ is alkyl.

In some embodiments, R₁ is cycloalkyl.

In some embodiments, R₁ is alkyl or cycloalkyl.

In some embodiments, R₁ is C₃-C₈ cycloalkyl.

In some embodiments, R₁ is unsubstituted or substituted arylalkyl, wherein the substituent is selected from halo and alkoxy.

In some embodiments, R₁ is heteroarylalkyl.

In some embodiments, R₂ and R_{2′} are each hydrogen.

In some embodiments, R₂ and R_{2′} are each alkyl.

In some embodiments, R₂ and R_{2′} are each independently hydrogen or alkyl.

In some embodiments, R_{X} is alkyl.

In some embodiments, R_{X} is methyl or isopropyl.

In some embodiments, R_{X} is alkoxy.

In some embodiments, R_{X} is methoxy or ethoxy.

In some embodiments, R_{X} is cycloalkyl.

In some embodiments, R_{X} is cyclopropyl.

In some embodiments, R_{X} at each occurrence independently is alkoxy or cycloalkyl.

In some embodiments, R_{X} at each occurrence independently is methoxy or cyclopropyl.

In some embodiments, R_{Y} is halo.

In some embodiments, R_{Y} is alkoxy.

In some embodiments, R_{Y} is alkylamino.

In some embodiments, R_{Z} is alkyl.

In some embodiments, R_{Z} is haloalkyl.

In some embodiments, R_{Z} is halo.

In some embodiments, R_{Z} is alkoxy.

In some embodiments, R_{Z} is cycloalkyl.

In some embodiments, R_{Z} at each occurrence independently is alkyl or haloalkyl.

In some embodiments, R_{Z} at each occurrence independently is methyl, ethyl, isopropyl, or trifluoromethyl.

In some embodiments. R_{Z} at each occurrence is independently cycloalkyl or haloalkyl.

In some embodiments, compounds of formula (I) are provided, wherein R_{X} is alkoxy or cycloalkyl, p is 0, R_{Z} is alkyl or haloalkyl, R₁ is alkyl and, R₂ and R_{2′} each hydrogen.

In some embodiments, compounds of formula (I) are provided, wherein ring X is pyrimidinyl, ring Y is pyridinyl and ring Z is imidazolyl.

In some embodiments, m is 1.

In some embodiments, m is 2.

In some embodiments, m is 1 or 2.

In some embodiments, n is 2.

In some embodiments, p is 0.

In some embodiments, p is 1.

In some embodiments, the present disclosure provides a compound selected from the group consisting of

| Comp. No. | IUPAC Name |
|---|---|
| 1. | 3-(4-cyclopropyl-6-methoxypyrimidin-5-yl)-1-methyl-5-(4-(1-methyl- |
| | 4-(trifluoromethyl)-1H-imidazol-2-yl)phenyl)-4,5,6,7-tetrahydro-1H- |
| | pyrazolo[4,3-c]pyridine |
| 2. | 3-(2-cyclopropyl-6-methoxyphenyl)-1-methyl-5-(4-(1-methyl-4- |
| | (trifluoromethyl)-1H-imidazol-2-yl)phenyl)-4,5,6,7-tetrahydro-1H- |
| | pyrazolo[4,3-c]pyridine |
| 3. | 3-(2-isopropylpyridin-3-yl)-1-methyl-5-(4-(1-methyl-4- |
| | (trifluoromethyl)-1H-imidazol-2-yl)phenyl)-4,5,6,7-tetrahydro-1H- |
| | pyrazolo[4,3-c]pyridine |
| 4. | 3-(2-isopropylpyridin-3-yl)-1-methyl-5-(6-(1-methyl-4- |
| | (trifluoromethyl)-1H-imidazol-2-yl)pyridin-3-yl)-4,5,6,7-tetrahydro- |
| | 1H-pyrazolo[4,3-c]pyridine |
| 5. | 3-(4-cyclopropyl-6-methoxypyrimidin-5-yl)-5-(4-(1-ethyl-4- |
| | (trifluoromethyl)-1H-imidazol-2-yl)phenyl)-1-methyl-4,5,6,7- |
| | tetrahydro-1H-pyrazolo[4,3-c]pyridine |
| 6. | 3-(4-cyclopropyl-6-methoxypyrimidin-5-yl)-5-(4-(1-isopropyl-4- |
| | (trifluoromethyl)-1H-imidazol-2-yl)phenyl)-1-methyl-4,5,6,7- |
| | tetrahydro-1H-pyrazolo[4,3-c]pyridine |
| 7. | 5-(4-(1-cyclopropyl-4-(trifluoromethyl)-1H-imidazol-2-yl)phenyl)-3- |
| | (4-cyclopropyl-6-methoxypyrimidin-5-yl)-1-methyl-4,5,6,7-tetrahydro- |
| | 1H-pyrazolo[4,3-c]pyridine |
| 8. | 5-(4-(1-cyclobutyl-4-(trifluoromethyl)-1H-imidazol-2-yl)phenyl)-3-(4- |
| | cyclopropyl-6-methoxypyrimidin-5-yl)-1-methyl-4,5,6,7-tetrahydro- |
| | 1H-pyrazolo[4,3-c]pyridine |
| 9. | 3-(4-cyclopropyl-6-methoxypyrimidin-5-yl)-5-(4-(1-(2-methoxyethyl)- |
| | 4-(trifluoromethyl)-1H-imidazol-2-yl)phenyl)-1-methyl-4,5,6,7- |
| | tetrahydro-1H-pyrazolo[4,3-c]pyridine |
| 10. | 2-(2-(4-(3-(4-cyclopropyl-6-methoxypyrimidin-5-yl)-1-methyl-1,4,6,7- |
| | tetrahydro-5H-pyrazolo[4,3-c]pyridin-5-yl)phenyl)-4-(trifluoromethyl)- |
| | 1H-imidazol-1-yl)-N,N-dimethylethan-1-amine |
| 11. | 3-(4-cyclopropyl-6-methoxypyrimidin-5-yl)-1-methyl-5-(4-(1-(1- |
| | methylazetidin-3-yl)-4-(trifluoromethyl)-1H-imidazol-2-yl)phenyl)- |
| | 4,5,6,7-tetrahydro-1H-pyrazolo[4,3-c]pyridine |
| 12. | 3-(4-cyclopropyl-6-methoxypyrimidin-5-yl)-1-methyl-5-(6-(1-methyl- |
| | 4-(trifluoromethyl)-1H-imidazol-2-yl)pyridin-3-yl)-4,5,6,7-tetrahydro- |
| | 1H-pyrazolo[4,3-c]pyridine |
| 13. | 3-(4-cyclopropyl-6-methoxypyrimidin-5-yl)-1-methyl-5-(5-(1-methyl- |
| | 4-(trifluoromethyl)-1H-imidazol-2-yl)pyridin-2-yl)-4,5,6,7-tetrahydro- |
| | 1H-pyrazolo[4,3-c]pyridine |
| 14. | 3-(4-cyclopropyl-6-methoxypyrimidin-5-yl)-5-(5-(1-ethyl-4- |
| | (trifluoromethyl)-1H-imidazol-2-yl)pyridin-2-yl)-1-methyl-4,5,6,7- |
| | tetrahydro-1H-pyrazolo[4,3-c]pyridine |
| 15. | 3-(4-cyclopropyl-6-methoxypyrimidin-5-yl)-5-(5-(1-isopropyl-4- |
| | (trifluoromethyl)-1H-imidazol-2-yl)pyridin-2-yl)-1-methyl-4,5,6,7- |
| | tetrahydro-1H-pyrazolo[4,3-c]pyridine |
| 16. | 5-(5-(1-cyclobutyl-4-(trifluoromethyl)-1H-imidazol-2-yl)pyridin-2-yl)- |
| | 3-(4-cyclopropyl-6-methoxypyrimidin-5-yl)-1-methyl-4,5,6,7- |
| | tetrahydro-1H-pyrazolo[4,3-c]pyridine |
| 17. | 3-(4-cyclopropyl-6-methoxypyrimidin-5-yl)-1-methyl-5-(4-(5-methyl- |
| | 3-(trifluoromethyl)-1H-pyrazol-1-yl)phenyl)-4,5,6,7-tetrahydro-1H- |
| | pyrazolo[4,3-c]pyridine |
| 18. | 3-(4-cyclopropyl-6-methoxypyrimidin-5-yl)-5-(4-(3,5-dimethyl-1H- |
| | pyrazol-1-yl)phenyl)-1-methyl-4,5,6,7-tetrahydro-1H-pyrazolo[4,3- |
| | c]pyridine |
| 19. | 3-(4-cyclopropyl-6-methoxypyrimidin-5-yl)-5-(4-(5-methoxy-3- |
| | (trifluoromethyl)-1H-pyrazol-1-yl)phenyl)-1-methyl-4,5,6,7- |
| | tetrahydro-1H-pyrazolo[4,3-c]pyridine |
| 20. | 3-(4-cyclopropyl-6-methoxypyrimidin-5-yl)-5-(4-(5-ethoxy-3- |
| | (trifluoromethyl)-1H-pyrazol-1-yl)phenyl)-1-methyl-4,5,6,7- |
| | tetrahydro-1H-pyrazolo[4,3-c]pyridine |
| 21. | 5-(4-(4-chloro-1-methyl-1H-imidazol-2-yl)phenyl)-3-(4-cyclopropyl-6- |
| | methoxypyrimidin-5-yl)-1-methyl-4,5,6,7-tetrahydro-1H-pyrazolo[4,3- |
| | c]pyridine |
| 22. | 3-(4-cyclopropyl-6-methoxypyrimidin-5-yl)-5-(2'-methoxy-[3,3′- |
| | bipyridin]-6-yl)-1-methyl-4,5,6,7-tetrahydro-1H-pyrazolo[4,3- |
| | c]pyridine |
| 23 | 4-(6-(3-(4-cyclopropyl-6-methoxypyrimidin-5-yl)-1-methyl-1,4,6,7- |
| | tetrahydro-5H-pyrazolo[4,3-c]pyridin-5-yl)pyridin-3-yl)-3,5- |
| | dimethylisoxazole |
| 24. | 3-(4-cyclopropyl-6-methoxypyrimidin-5-yl)-1-ethyl-5-(4-(1-methyl-4- |
| | (trifluoromethyl)-1H-imidazol-2-yl)phenyl)-4,5,6,7-tetrahydro-1H- |
| | pyrazolo[4,3-c]pyridine |
| 25. | 3-(4-cyclopropyl-6-methoxypyrimidin-5-yl)-1-ethyl-5-(6-(1-methyl-4- |
| | (trifluoromethyl)-1H-imidazol-2-yl)pyridin-3-yl)-4,5,6,7-tetrahydro- |
| | 1H-pyrazolo[4,3-c]pyridine |
| 26. | 3-(4-cyclopropyl-6-methoxypyrimidin-5-yl)-1-ethyl-5-(5-(1-methyl-4- |
| | (trifluoromethyl)-1H-imidazol-2-yl)pyridin-2-yl)-4,5,6,7-tetrahydro- |
| | 1H-pyrazolo[4,3-c]pyridine |
| 27. | 3-(4-cyclopropyl-6-methoxypyrimidin-5-yl)-1-isopropyl-5-(4-(1- |
| | methyl-4-(trifluoromethyl)-1H-imidazol-2-yl)phenyl)-4,5,6,7- |
| | tetrahydro-1H-pyrazolo[4,3-c]pyridine |
| 28. | 3-(4-cyclopropyl-6-methoxypyrimidin-5-yl)-1-isopropyl-5-(6-(1- |
| | methyl-4-(trifluoromethyl)-1H-imidazol-2-yl)pyridin-3-yl)-4,5,6,7- |
| | tetrahydro-1H-pyrazolo[4,3-c]pyridine |
| 29. | 3-(4-cyclopropyl-6-methoxypyrimidin-5-yl)-1-isopropyl-5-(5-(1- |
| | methyl-4-(trifluoromethyl)-1H-imidazol-2-yl)pyridin-2-yl)-4,5,6,7- |
| | tetrahydro-1H-pyrazolo[4,3-c]pyridine |
| 30. | 1-cyclopropyl-3-(4-cyclopropyl-6-methoxypyrimidin-5-yl)-5-(4-(1- |
| | methyl-4-(trifluoromethyl)-1H-imidazol-2-yl)phenyl)-4,5,6,7- |
| | tetrahydro-1H-pyrazolo[4,3-c]pyridine |
| 31. | 1-cyclopropyl-3-(4-cyclopropyl-6-methoxypyrimidin-5-yl)-5-(6-(1- |
| | methyl-4-(trifluoromethyl)-1H-imidazol-2-yl)pyridin-3-yl)-4,5,6,7- |
| | tetrahydro-1H-pyrazolo[4,3-c]pyridine |
| 32. | 1-cyclopropyl-3-(4-cyclopropyl-6-methoxypyrimidin-5-yl)-5-(5-(1- |
| | methyl-4-(trifluoromethyl)-1H-imidazol-2-yl)pyridin-2-yl)-4,5,6,7- |
| | tetrahydro-1H-pyrazolo[4,3-c]pyridine |
| 33. | 1-cyclobutyl-3-(4-cyclopropyl-6-methoxypyrimidin-5-yl)-5-(4-(1- |
| | methyl-4-(trifluoromethyl)-1H-imidazol-2-yl)phenyl)-4,5,6,7- |
| | tetrahydro-1 H-pyrazolo[4,3-c]pyridine |
| 34. | 1-cyclobutyl-3-(4-cyclopropyl-6-methoxypyrimidin-5-yl)-5-(6-(1- |
| | methyl-4-(trifluoromethyl)-1H-imidazol-2-yl)pyridin-3-yl)-4,5,6,7- |
| | tetrahydro-1H-pyrazolo[4,3-c]pyridine |
| 35. | 1-cyclobutyl-3-(4-cyclopropyl-6-methoxypyrimidin-5-yl)-5-(5-(1- |
| | methyl-4-(trifluoromethyl)-1H-imidazol-2-yl)pyridin-2-yl)-4,5,6,7- |
| | tetrahydro-1H-pyrazolo[4,3-c]pyridine |
| 36. | 1-benzyl-3-(4-cyclopropyl-6-methoxypyrimidin-5-yl)-5-(4-(1-methyl- |
| | 4-(trifluoromethyl)-1H-imidazol-2-yl)phenyl)-4,5,6,7-tetrahydro-1H- |
| | pyrazolo[4,3-c]pyridine |
| 37. | 1-benzyl-3-(4-cyclopropyl-6-methoxypyrimidin-5-yl)-5-(5-(1-methyl- |
| | 4-(trifluoromethyl)-1H-imidazol-2-yl)pyridin-2-yl)-4,5,6,7-tetrahydro- |
| | 1H-pyrazolo[4,3-c]pyridine |
| 38. | 3-(4-cyclopropyl-6-methoxypyrimidin-5-yl)-1,7-dimethyl-5-(4-(1- |
| | methyl-4-(trifluoromethyl)-1H-imidazol-2-yl)phenyl)-4,5,6,7- |
| | tetrahydro-1H-pyrazolo[4,3-c]pyridine |
| 39. | 3-(4-cyclopropyl-6-methoxypyrimidin-5-yl)-1,7-dimethyl-5-(6-(1- |
| | methyl-4-(trifluoromethyl)-1H-imidazol-2-yl)pyridin-3-yl)-4,5,6,7- |
| | tetrahydro-1H-pyrazolo[4,3-c]pyridine |
| 40. | 3-(4-cyclopropyl-6-methoxypyrimidin-5-yl)-1,7-dimethyl-5-(5-(1- |
| | methyl-4-(trifluoromethyl)-1H-imidazol-2-yl)pyridin-2-yl)-4,5,6,7- |
| | tetrahydro-1H-pyrazolo[4,3-c]pyridine |
| 41. | 3-(4-cyclopropyl-6-methoxypyrimidin-5-yl)-1,7,7-trimethyl-5-(4-(1- |
| | methyl-4-(trifluoromethyl)-1H-imidazol-2-yl)phenyl)-4,5,6,7- |
| | tetrahydro-1H-pyrazolo[4,3-c]pyridine |
| 42. | 3-(4-cyclopropyl-6-methoxypyrimidin-5-yl)-1,7,7-trimethyl-5-(6-(1- |
| | methyl-4-(trifluoromethyl)-1H-imidazol-2-yl)pyridin-3-yl)-4,5,6,7- |
| | tetrahydro-1H-pyrazolo[4,3-c]pyridine |
| 43. | 3-(4-cyclopropyl-6-methoxypyrimidin-5-yl)-1,7,7-trimethyl-5-(5-(1- |
| | methyl-4-(trifluoromethyl)-1H-imidazol-2-yl)pyridin-2-yl)-4,5,6,7- |
| | tetrahydro-1H-pyrazolo[4,3-c]pyridine |
| 44. | 3-(4-cyclopropyl-6-methoxypyrimidin-5-yl)-5-(3-fluoro-4-(1-methyl-4- |
| | (trifluoromethyl)-1H-imidazol-2-yl)phenyl)-1-methyl-4,5,6,7- |
| | tetrahydro-1H-pyrazolo[4,3-c]pyridine |
| 45. | 5-(3-chloro-4-(1-methyl-4-(trifluoromethyl)-1H-imidazol-2-yl)phenyl)- |
| | 3-(4-cyclopropyl-6-methoxypyrimidin-5-yl)-1-methyl-4,5,6,7- |
| | tetrahydro-1H-pyrazolo[4,3-c]pyridine |
| 46. | 3-(4-cyclopropyl-6-methoxypyrimidin-5-yl)-5-(3-methoxy-4-(1- |
| | methyl-4-(trifluoromethyl)-1H-imidazol-2-yl)phenyl)-1-methyl- |
| | 4,5,6,7-tetrahydro-1H-pyrazolo[4,3-c]pyridine |
| 47. | 3-(4-cyclopropyl-6-methoxypyrimidin-5-yl)-5-(3-ethoxy-4-(1-methyl- |
| | 4-(trifluoromethyl)-1H-imidazol-2-yl)phenyl)-1-methyl-4,5,6,7- |
| | tetrahydro-1H-pyrazolo[4,3-c]pyridine |
| 48. | 5-(3-(4-cyclopropyl-6-methoxypyrimidin-5-yl)-1-methyl-1,4,6,7- |
| | tetrahydro-5H-pyrazolo[4,3-c]pyridin-5-yl)-N,N-dimethyl-2-(1-methyl- |
| | 4-(trifluoromethyl)-1H-imidazol-2-yl)aniline |
| 49. | 3-(4-cyclopropyl-6-methoxypyrimidin-5-yl)-1-methyl-5-(5-(1-methyl- |
| | 4-(trifluoromethyl)-1H-imidazol-2-yl)thiophen-3-yl)-4,5,6,7- |
| | tetrahydro-1H-pyrazolo[4,3-c]pyridine |
| 50. | 4-(3-(4-cyclopropyl-6-methoxypyrimidin-5-yl)-1-methyl-1,4,6,7- |
| | tetrahydro-5H-pyrazolo[4,3-c]pyridin-5-yl)-2-(1-methyl-4- |
| | (trifluoromethyl)-1H-imidazol-2-yl)thiazole |
| 51. | 3-(4-cyclopropyl-6-methoxypyrimidin-5-yl)-1-methyl-5-(2-(5-methyl- |
| | 3-(trifluoromethyl)-1H-pyrazol-1-yl)pyrimidin-5-yl)-4,5,6,7- |
| | tetrahydro-1H-pyrazolo[4,3-c]pyridine |
| 52. | 3-(4-cyclopropyl-6-methoxypyrimidin-5-yl)-1-methyl-5-(5-(1-methyl- |
| | 4-(trifluoromethyl)-1H-imidazol-2-yl)pyrazin-2-yl)-4,5,6,7-tetrahydro- |
| | 1H-pyrazolo[4,3-c]pyridine |
| 53. | 3-(4-cyclopropyl-6-methoxypyrimidin-5-yl)-5-(4-(1,5-dimethyl- |
| | 4,5,6,7-tetrahydro-1H-pyrazolo[4,3-c]pyridin-3-yl)phenyl)-1-methyl- |
| | 4,5,6,7-tetrahydro-1H-pyrazolo[4,3-c]pyridine |
| 54. | 3-(4-cyclopropyl-6-methoxypyrimidin-5-yl)-5-(4-(1-methyl-4- |
| | (trifluoromethyl)-1H-imidazol-2-yl)phenyl)-4,5,6,7-tetrahydro-1H- |
| | pyrazolo[4,3-c]pyridine |
| 55. | 5-(4-(1-(azetidin-3-yl)-4-(trifluoromethyl)-1H-imidazol-2-yl)phenyl)- |
| | 3-(4-cyclopropyl-6-methoxypyrimidin-5-yl)-1-methyl-4,5,6,7- |
| | tetrahydro-1H-pyrazolo[4,3-c]pyridine |
| 56. | 3-(4-cyclopropyl-6-methoxypyrimidin-5-yl)-1-methyl-5-(4-(1-methyl- |
| | 4,5,6,7-tetrahydro-1H-pyrazolo[4,3-c]pyridin-3-yl)phenyl)-4,5,6,7- |
| | tetrahydro-1H-pyrazolo[4,3-c]pyridine |
| 57. | 3-(4-cyclopropyl-6-methoxypyrimidin-5-yl)-5-(3-ethyl-4-(1-methyl-4- |
| | (trifluoromethyl)-1H-imidazol-2-yl)phenyl)-1-methyl-4,5,6,7- |
| | tetrahydro-1H-pyrazolo[4,3-c]pyridine |
| 58. | 3-(4-cyclopropyl-6-ethoxypyrimidin-5-yl)-1-methyl-5-(4-(1-methyl-4- |
| | (trifluoromethyl)-1H-imidazol-2-yl)phenyl)-4,5,6,7-tetrahydro-1H- |
| | pyrazolo[4,3-c]pyridine |
| 59. | 3-(4-cyclopropyl-6-methoxypyrimidin-5-yl)-1-methyl-5-(5-(1-methyl- |
| | 4-(trifluoromethyl)-1H-imidazol-2-yl)pyrimidin-2-yl)-4,5,6,7- |
| | tetrahydro-1H-pyrazolo[4,3-c]pyridine |
| 60. | 3-(4-cyclopropyl-2-methoxypyridin-3-yl)-1-methyl-5-(4-(1-methyl-4- |
| | (trifluoromethyl)-1H-imidazol-2-yl)phenyl)-4,5,6,7-tetrahydro-1H- |
| | pyrazolo[4,3-c]pyridine |
| 61. | 3-(1-isopropyl-4-methyl-1H-pyrazol-5-yl)-1-methyl-5-(4-(1-methyl-4- |
| | (trifluoromethyl)-1H-imidazol-2-yl)phenyl)-4,5,6,7-tetrahydro-1H- |
| | pyrazolo[4,3-c]pyridine |
| 62. | 3-(1-isopropyl-4-methyl-1H-pyrazol-5-yl)-1-methyl-5-(6-(1-methyl-4- |
| | (trifluoromethyl)-1H-imidazol-2-yl)pyridin-3-yl)-4,5,6,7-tetrahydro- |
| | 1H-pyrazolo[4,3-c]pyridine |
| 63. | 3-(4-cyclopropyl-6-methoxypyrimidin-5-yl)-1-methyl-5-(6-(1-methyl- |
| | 4-(trifluoromethyl)-1H-imidazol-2-yl)pyridazin-3-yl)-4,5,6,7- |
| | tetrahydro-1H-pyrazolo[4,3-c]pyridine |
| 64. | 3-(4-cyclopropyl-6-methoxypyrimidin-5-yl)-1-methyl-5-(2-(1-methyl- |
| | 4-(trifluoromethyl)-1H-imidazol-2-yl)pyrimidin-5-yl)-4,5,6,7- |
| | tetrahydro-1H-pyrazolo[4,3-c]pyridine |
| 65. | 2-(3-(4-cyclopropyl-6-methoxypyrimidin-5-yl)-5-(5-(1-methyl-4- |
| | (trifluoromethyl)-1H-imidazol-2-yl)pyridin-2-yl)-4,5,6,7-tetrahydro- |
| | 1H-pyrazolo[4,3-c]pyridin-1-yl)-N,N-dimethylethan-1-amine |
| 66. | 3-(4-cyclopropyl-6-methoxypyrimidin-5-yl)-5-(5-(1-methyl-4- |
| | (trifluoromethyl)-1H-imidazol-2-yl)pyridin-2-yl)-1-(1-methylazetidin- |
| | 3-yl)-4,5,6,7-tetrahydro-1H-pyrazolo[4,3-c]pyridine |
| 67. | 3-(4-cyclopropyl-6-methoxypyrimidin-5-yl)-5-(5-(1-methyl-4- |
| | (trifluoromethyl)-1H-imidazol-2-yl)pyridin-2-yl)-1-(oxetan-3-yl)- |
| | 4,5,6,7-tetrahydro-1H-pyrazolo[4,3-c]pyridine |
| 68. | 3-(4-cyclopropyl-6-methoxypyrimidin-5-yl)-5-(5-(1-methyl-4- |
| | (trifluoromethyl)-1H-imidazol-2-yl)pyridin-2-yl)-1-(pyridin-2- |
| | ylmethyl)-4,5,6,7-tetrahydro-1H-pyrazolo[4,3-c]pyridine |
| 69. | 3-(4-cyclopropyl-6-methoxypyrimidin-5-yl)-5-(5-(1-methyl-4- |
| | (trifluoromethyl)-1H-imidazol-2-yl)pyridin-2-yl)-1-(pyridin-3- |
| | ylmethyl)-4,5,6,7-tetrahydro-1H-pyrazolo[4,3-c]pyridine |
| 70. | 3-(4-cyclopropyl-6-methoxypyrimidin-5-yl)-5-(5-(1-methyl-4- |
| | (trifluoromethyl)-1H-imidazol-2-yl)pyridin-2-yl)-1-(pyrimidin-2- |
| | ylmethyl)-4,5,6,7-tetrahydro-1H-pyrazolo[4,3-c]pyridine |
| 71. | 3-(4-cyclopropyl-6-methoxypyrimidin-5-yl)-5-(5-(1-methyl-4- |
| | (trifluoromethyl)-1H-imidazol-2-yl)pyridin-2-yl)-1-(pyrazin-2- |
| | ylmethyl)-4,5,6,7-tetrahydro-1H-pyrazolo[4,3-c]pyridine |
| 72. | 3-(4-cyclopropyl-6-methoxypyrimidin-5-yl)-5-(5-(1-methyl-4- |
| | (trifluoromethyl)-1H-imidazol-2-yl)pyridin-2-yl)-1-(pyridazin-3- |
| | ylmethyl)-4,5,6,7-tetrahydro-1H-pyrazolo[4,3-c]pyridine |
| 73. | 3-(4-cyclopropyl-6-methoxypyrimidin-5-yl)-1-(4-fluorobenzyl)-5-(5- |
| | (1-methyl-4-(trifluoromethyl)-1H-imidazol-2-yl)pyridin-2-yl)-4,5,6,7- |
| | tetrahydro-1H-pyrazolo[4,3-c]pyridine |
| 74. | 3-(4-cyclopropyl-6-methoxypyrimidin-5-yl)-1-(3-fluorobenzyl)-5-(5- |
| | (1-methyl-4-(trifluoromethyl)-1H-imidazol-2-yl)pyridin-2-yl)-4,5,6,7- |
| | tetrahydro-1H-pyrazolo[4,3-c]pyridine |
| 75. | 3-(4-cyclopropyl-6-methoxypyrimidin-5-yl)-1-(2-fluorobenzyl)-5-(5- |
| | (1-methyl-4-(trifluoromethyl)-1H-imidazol-2-yl)pyridin-2-yl)-4,5,6,7- |
| | tetrahydro-1H-pyrazolo[4,3-c]pyridine |
| 76. | 3-(4-cyclopropyl-6-methoxypyrimidin-5-yl)-1-(4-chlorobenzyl)-5-(5- |
| | (1-methyl-4-(trifluoromethyl)-1H-imidazol-2-yl)pyridin-2-yl)-4,5,6,7- |
| | tetrahydro-1H-pyrazolo[4,3-c]pyridine |
| 77. | 3-(4-cyclopropyl-6-methoxypyrimidin-5-yl)-1-(3-chlorobenzyl)-5-(5- |
| | (1-methyl-4-(trifluoromethyl)-1H-imidazol-2-yl)pyridin-2-yl)-4,5,6,7- |
| | tetrahydro-1H-pyrazolo[4,3-c]pyridine |
| 78. | 3-(4-cyclopropyl-6-methoxypyrimidin-5-yl)-1-(2-chlorobenzyl)-5-(5- |
| | (1-methyl-4-(trifluoromethyl)-1H-imidazol-2-yl)pyridin-2-yl)-4,5,6,7- |
| | tetrahydro-1H-pyrazolo[4,3-c]pyridine |
| 79. | 3-(4-cyclopropyl-6-methoxypyrimidin-5-yl)-1-(4-methoxybenzyl)-5- |
| | (5-(1-methyl-4-(trifluoromethyl)-1H-imidazol-2-yl)pyridin-2-yl)- |
| | 4,5,6,7-tetrahydro-1H-pyrazolo[4,3-c]pyridine |
| 80. | 3-(4-cyclopropyl-6-methoxypyrimidin-5-yl)-1-(3-methoxybenzyl)-5- |
| | (5-(1-methyl-4-(trifluoromethyl)-1H-imidazol-2-yl)pyridin-2-yl)- |
| | 4,5,6,7-tetrahydro-1H-pyrazolo[4,3-c]pyridine |
| 81. | 3-(4-cyclopropyl-6-methoxypyrimidin-5-yl)-1-(2-methoxybenzyl)-5- |
| | (5-(1-methyl-4-(trifluoromethyl)-1H-imidazol-2-yl)pyridin-2-yl)- |
| | 4,5,6,7-tetrahydro-1H-pyrazolo[4,3-c]pyridine |

or a pharmaceutically acceptable salt or a stereoisomer thereof.

In some embodiments, a compound of formula (II) is provided:

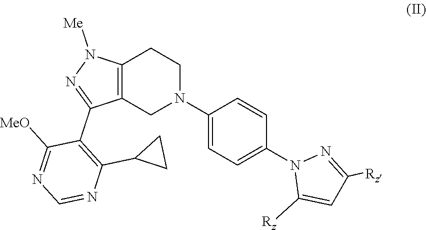

or a pharmaceutically acceptable salt thereof or a stereoisomer thereof, wherein:
R_{z} is selected from the group consisting of alkyl, alkoxy and dialkylamino; and
R_{z′} is CH₃ or CF₃.

In some embodiments of the compound of formula (II), R_{z′} is CF₃.

In some embodiments, the compound of formula (II) is selected from the group consisting of:

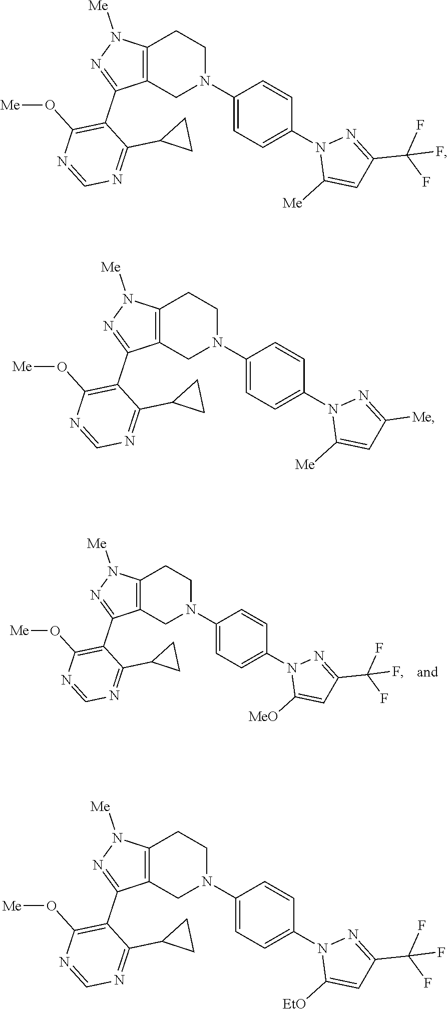 [SMILES: COc1ncnc(C2CC2)c1-c1nn(C)c2c1CN(c1ccc(-n3nc(C(F)(F)F)cc3C)cc1)CC2 ; COc1ncnc(C2CC2)c1-c1nn(C)c2c1CN(c1ccc(-n3nc(C)cc3C)cc1)CC2 ; COc1ncnc(C2CC2)c1-c1nn(C)c2c1CN(c1ccc(-n3nc(C(F)(F)F)cc3OC)cc1)CC2 ; "and" ; CCOc1cc(C(F)(F)F)nn1-c1ccc(N2CCc3c(c(-c4c(OC)ncnc4C4CC4)nn3C)C2)cc1]

or a pharmaceutically acceptable salt thereof or a stereoisomer thereof.

In some embodiments, a compound of formula (III) is provided:

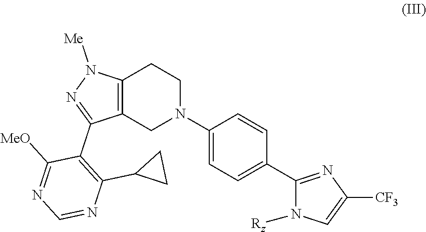

or a pharmaceutically acceptable salt thereof or a stereoisomer thereof, wherein:
R_{z} is selected from the group consisting of ethyl, isopropyl, C₃-C₅ cycloalkyl, alkoxyalkyl, dialkylaminoalkyl, and C₃-C₅ heterocycloalkyl optionally substituted with methyl.

In some embodiments, the compound of formula (III) is selected from the group consisting of:

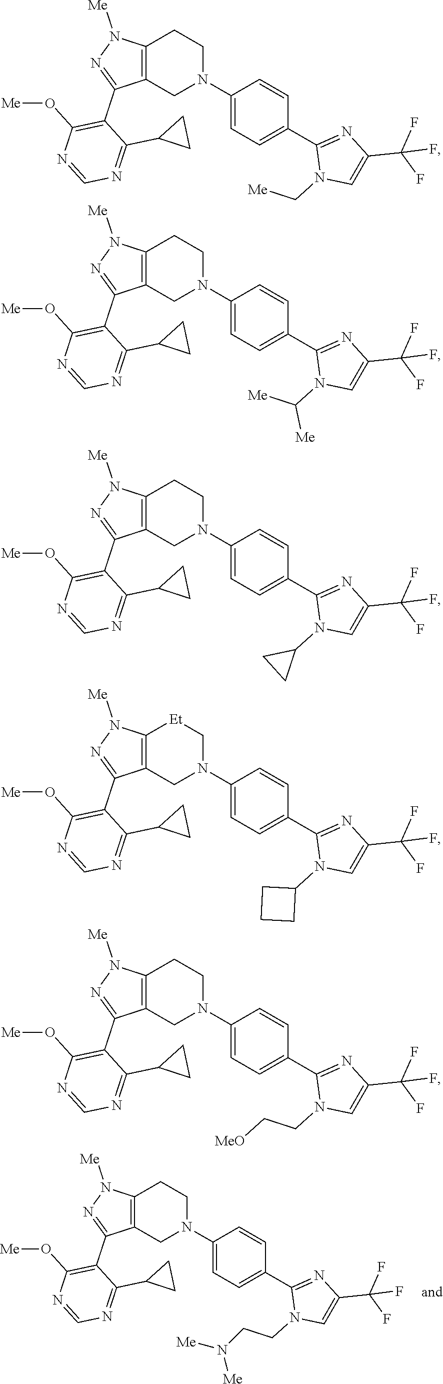

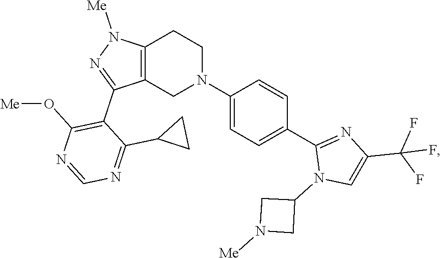 [SMILES: COc1ncnc(C2CC2)c1-c1nn(C)c2c1CN(c1ccc(-c3nc(C(F)(F)F)cn3C3CN(C)C3)cc1)CC2]

or a pharmaceutically acceptable salt thereof or a stereoisomer thereof.

In some embodiments, a compound of formula (IV) is provided:

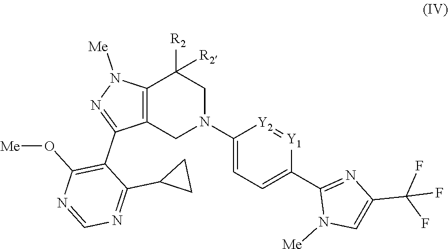

or a pharmaceutically acceptable salt thereof or a stereoisomer thereof, wherein:
R₂ and R_{2′} are independently selected from H, methyl, and ethyl, and at least one of R₂ and R_{2′} is methyl or ethyl; and
Y₁ and Y₂ are CH or N, wherein at least one of Y₁ and Y₂ is CH.

In some embodiments, the compound of formula (IV) is selected from the group consisting of:

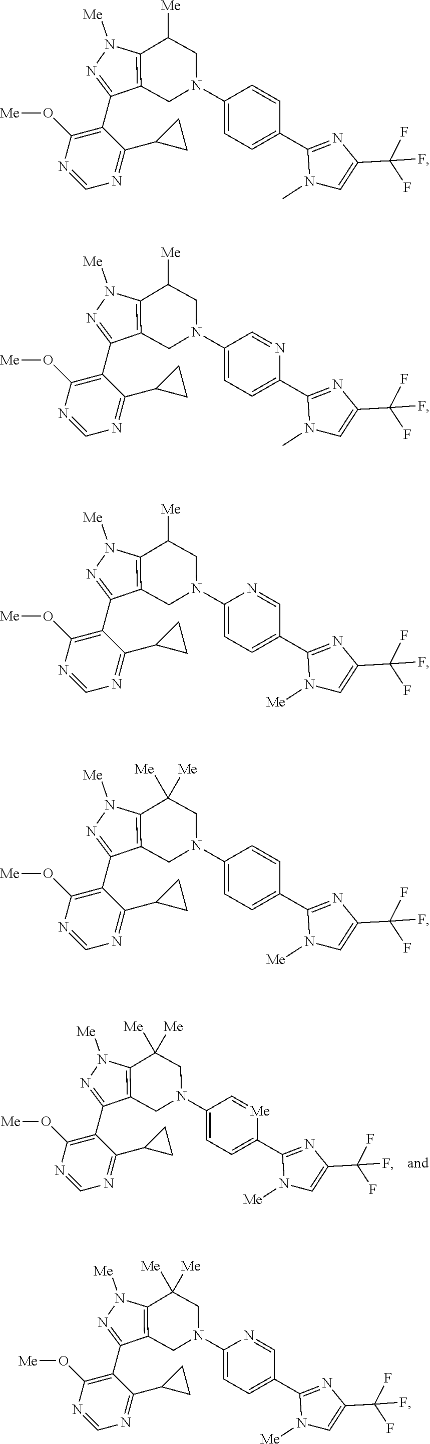

or a pharmaceutically acceptable salt thereof or a stereoisomer thereof.

In some embodiments, a compound of formula (V) is provided:

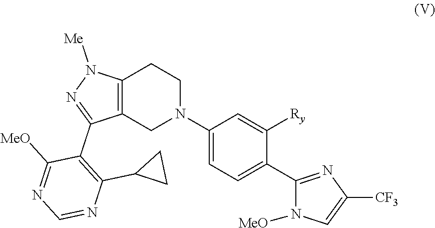

or a pharmaceutically acceptable salt thereof or a stereoisomer thereof, wherein:
R_{y} is selected from the group consisting of Cl, Br, alkoxy, and dialkylamino.

In some embodiments, the compound of formula (V) is selected from the group consisting of:

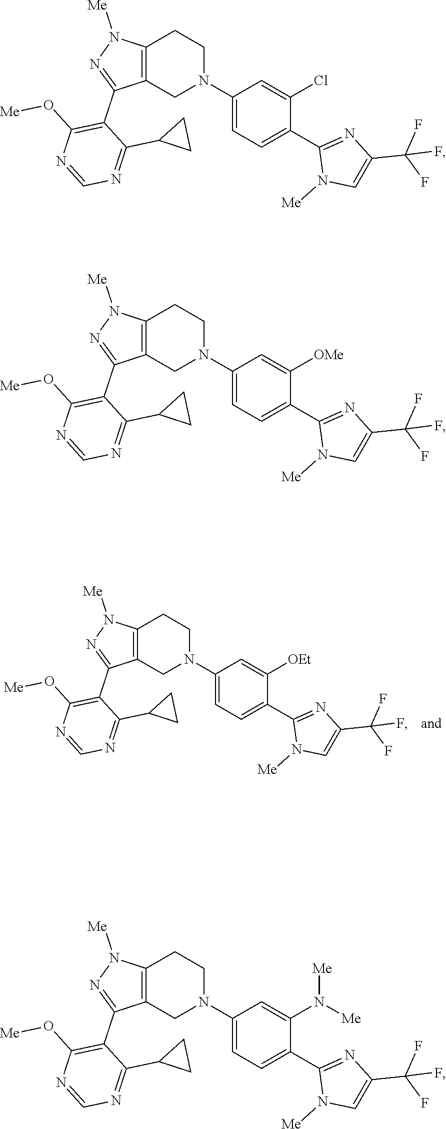 [SMILES: COc1ncnc(C2CC2)c1-c1nn(C)c2c1CN(c1ccc(-c3nc(C(F)(F)F)cn3C)c(Cl)c1)CC2 ; COc1cc(N2CCc3c(c(-c4c(OC)ncnc4C4CC4)nn3C)C2)ccc1-c1nc(C(F)(F)F)cn1C ; CCOc1cc(N2CCc3c(c(-c4c(OC)ncnc4C4CC4)nn3C)C2)ccc1-c1nc(C(F)(F)F)cn1C ; "and" ; COc1ncnc(C2CC2)c1-c1nn(C)c2c1CN(c1ccc(-c3nc(C(F)(F)F)cn3C)c(N(C)C)c1)CC2]

or a pharmaceutically acceptable salt thereof or a stereoisomer thereof.

In some embodiments, a compound of formula (VI) is provided:

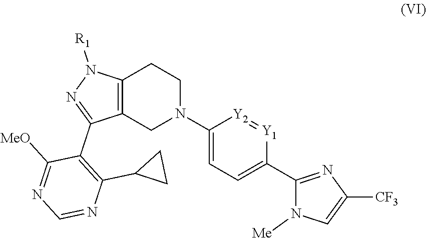

or a pharmaceutically acceptable salt thereof or a stereoisomer thereof, wherein:
R₁ is selected from the group consisting of C₂-C₄ alkyl, C₃-C₅ cycloalkyl, and arylalkyl; and
Y₁ and Y₂ are CH or N, wherein at least one of Y₁ and Y₂ is CH;
with the proviso that the compound of formula (VI) is not 3-(4-cyclopropyl-6-methoxypyrimidin-5-yl)-1-ethyl-5-(5-(1-methyl-4-(trifluoromethyl)-1H-imidazol-2-yl)pyridin-2-yl)-4,5,6,7-tetrahydro-1H-pyrazolo[4,3-c]pyridine.

In some embodiments, the compound of formula (VI) is selected from the group consisting of:

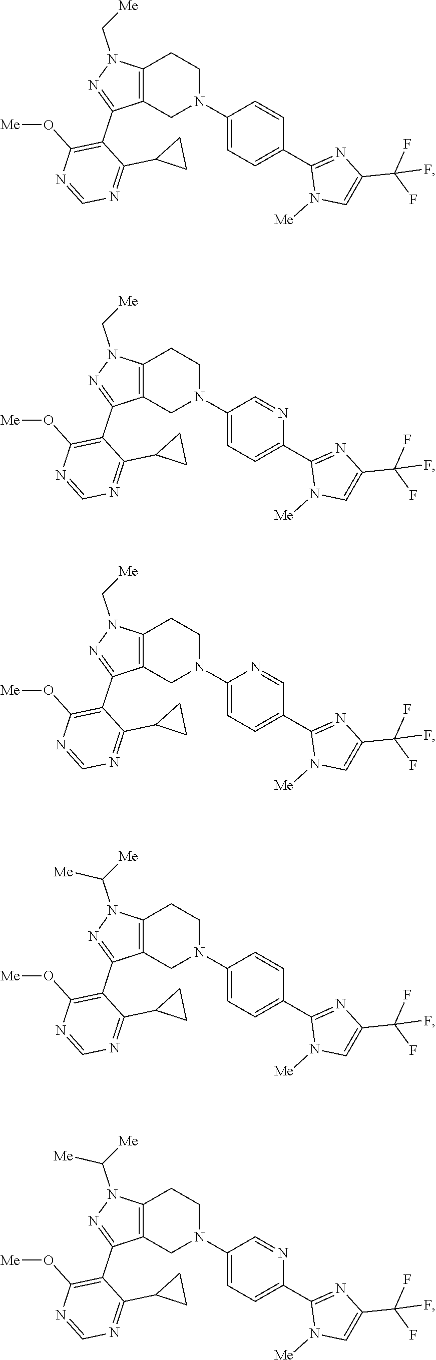 [SMILES: CCn1nc(-c2c(OC)ncnc2C2CC2)c2c1CCN(c1ccc(-c3nc(C(F)(F)F)cn3C)cc1)C2 ; CCn1nc(-c2c(OC)ncnc2C2CC2)c2c1CCN(c1ccc(-c3nc(C(F)(F)F)cn3C)nc1)C2 ; CCn1nc(-c2c(OC)ncnc2C2CC2)c2c1CCN(c1ccc(-c3nc(C(F)(F)F)cn3C)cn1)C2 ; COc1ncnc(C2CC2)c1-c1nn(C(C)C)c2c1CN(c1ccc(-c3nc(C(F)(F)F)cn3C)cc1)CC2 ; COc1ncnc(C2CC2)c1-c1nn(C(C)C)c2c1CN(c1ccc(-c3nc(C(F)(F)F)cn3C)nc1)CC2]

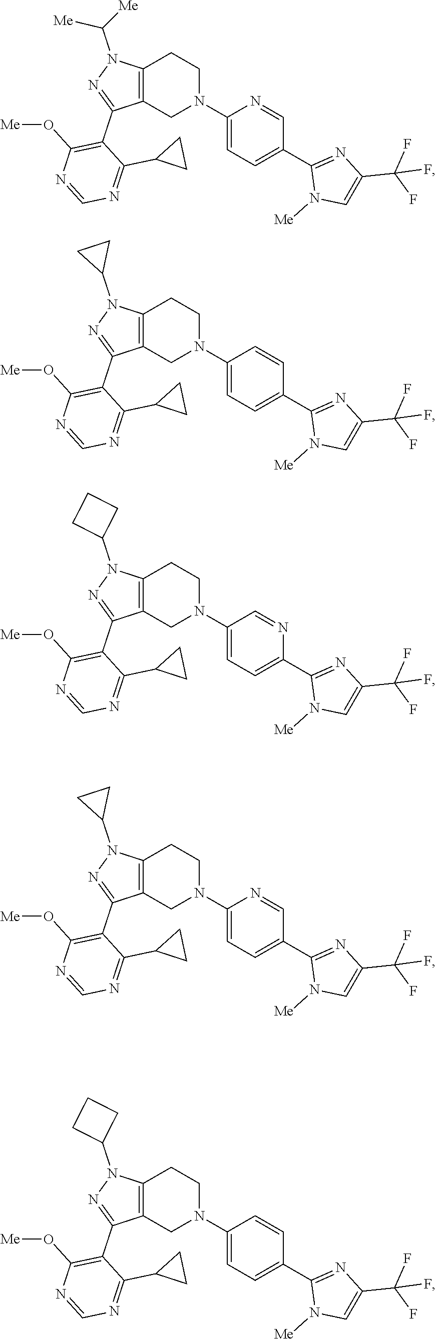

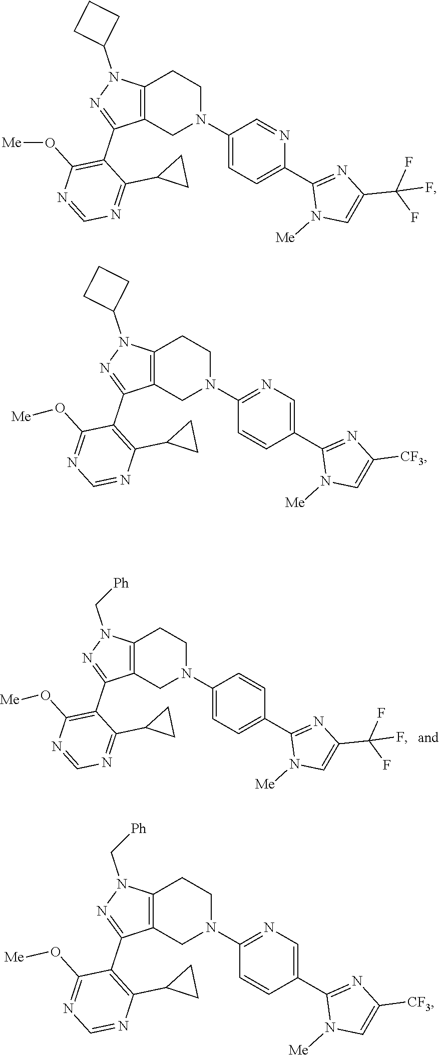 [SMILES: COc1ncnc(C2CC2)c1-c1nn(C2CCC2)c2c1CN(c1ccc(-c3nc(C(F)(F)F)cn3C)nc1)CC2 ; COc1ncnc(C2CC2)c1-c1nn(C2CCC2)c2c1CN(c1ccc(-c3nc(C(F)(F)F)cn3C)cn1)CC2 ; COc1ncnc(C2CC2)c1-c1nn(Cc2ccccc2)c2c1CN(c1ccc(-c3nc(C(F)(F)F)cn3C)cc1)CC2 ; COc1ncnc(C2CC2)c1-c1nn(Cc2ccccc2)c2c1CN(c1ccc(-c3nc(C(F)(F)F)cn3C)cn1)CC2]

or a pharmaceutically acceptable salt thereof or a stereoisomer thereof.

In some embodiments, a compound of formula (VII) is provided:

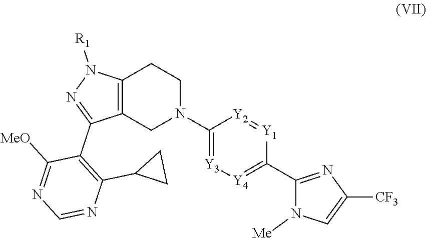

or a pharmaceutically acceptable salt thereof or a stereoisomer thereof, wherein:
R₁ is selected from the group consisting of alkoxyalkyl, dialkylaminoalkyl, cycloalkyl, heterocycloalkyl, arylalkyl substituted by one or more halogen, arylalkyl substituted by one or more alkoxy, and —CH₂-heteroaryl; and
Y₁, Y₂, Y₃, and Y₄ are CH or N.

In some embodiments, the present disclosure provides a pharmaceutical composition comprising a compound of formula (I), or a pharmaceutically acceptable salt thereof or a stereoisomer thereof as described herein and at least one pharmaceutically acceptable excipient (such as a pharmaceutically acceptable carrier or diluent). Preferably, the pharmaceutical composition comprises a therapeutically effective amount of at least one compound described herein (e.g., in unit dose form). The compounds described in the present disclosure may be associated with a pharmaceutically acceptable excipient (such as a carrier or a diluent) or be diluted by a carrier or enclosed within a carrier which can be in the form of a capsule, sachet, paper or other container.

The compounds of the disclosure are typically administered in the form of a pharmaceutical composition. Such compositions can be prepared using procedures well known in the pharmaceutical art and comprise at least one compound of the present disclosure. The pharmaceutical composition of the present disclosure comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents, wetting agents, suspending agents, solvents, sweetening agents, flavoring agents, colorants and the like.

Examples of suitable carriers include, but are not limited to, water, salt solutions, alcohols, polyethylene glycols/polyoxyethylenes, peanut oil, olive oil, gelatin, sugars (e.g., lactose, sucrose, and the like) terra alba, sucrose, dextrin, magnesium carbonate, amylose, talc, agar, pectin, acacia, lower alkyl ethers of cellulose, silicic acid, fatty acids and salts thereof (e.g., stearic acid, magnesium stearate, and the like), fatty acid amines, fatty acid monoglycerides and diglycerides, and fatty acid esters.

Administration of the compounds of the disclosure, in pure form or in an appropriate pharmaceutical composition, can be carried out using any of the accepted routes of administration of pharmaceutical compositions. The route of administration may be any route which effectively transports the active compound of the present disclosure to the appropriate or desired site of action. Suitable routes of administration include, but are not limited to, oral, parenteral, and inhalation routes, e.g., nasal, buccal, dermal, intradermal, transdermal, percutaneous, transmucosal, transnasal, transpulmonary, rectal, subcutaneous, intravenous, intraurethral, intramuscular or topical.

The pharmaceutical compositions may be in conventional forms, for example, tablets, capsules, solutions, suspensions, injectables or products for topical application. Further, the pharmaceutical composition of the present disclosure may be formulated so as to provide a desired release profile.

Solid oral formulations include, but are not limited to, tablets, capsules (soft or hard gelatin), dragees (containing the active ingredient in powder or pellet form), troches and lozenges.

Liquid formulations include, but are not limited to, syrups, emulsions and sterile injectable liquids, such as suspensions or solutions.

Topical dosage forms of the compounds include ointments, pastes, creams, lotions, powders, solutions, eye or ear drops, impregnated dressings and may contain appropriate conventional additives such as preservatives, solvents to assist drug penetration.

The pharmaceutical compositions of the present disclosure may be prepared by conventional techniques known in literature.

Suitable doses of the compounds for use in treating the diseases or disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms and suitable pharmaceutical excipients can also be well used and adjusted by those skilled in the art. All changes and modifications are envisioned within the scope of the present disclosure.

Compounds of the present disclosure are (e.g., compounds of formula (I)) are useful as USP1 inhibitors. In some embodiments, the present disclosure provides a method of inhibiting USP1 in a subject, comprising administering to the subject in need thereof an effective amount (e.g., a therapeutically effective amount) of a compound of the present disclosure. In some embodiments, the pharmaceutical composition comprising the compound of formula (I) is for use in treating a subject suffering from a disease or condition associated with USP1. In some embodiments, the present disclosure provides pharmaceutical composition for use in treating and/or preventing a disease and/or disorder responsive to the inhibition of USP1 proteins and USP1 activity.

In some embodiments, the disclosure provides the use of the compounds as described above in the treatment and prevention of diseases and/or disorder responsive to the inhibition of USP1 proteins and USP1 activity. In some embodiments, use of the compound or a pharmaceutically acceptable salt thereof, in treating and/or preventing a disease is provided, for which the symptoms thereof are treated, improved, diminished and/or prevented by inhibition of USP1. In some embodiments, the USP1 mediated disorder and/or disease or condition is cancer. Accordingly, compounds of formula (I) for use in the treatment of cancer are also provided provided, as well as use of the compounds of the present disclosure in the manufacture of medicaments (e.g., a medicament for the treatment of diseases and/or disorder responsive to the inhibition of USP1 proteins and USP1 activity such as cancer).

In some embodiments, the disclosure provides a method of treating a disease or disorder mediated by USP1 in a subject comprising administering to the subject a therapeutically effective amount of a compound of formula (I) or a pharmaceutically acceptable salt or a stereoisomer thereof. In some embodiments, the disease or disorder is cancer.

In some embodiments, the subject is a mammal including human.

In some embodiments, the cancer is selected from the group consisting of a hematological cancer, a lymphatic cancer, a DNA damage repair pathway deficient cancer, a homologous-recombination deficient cancer, a cancer comprising cancer cells with a mutation in a gene encoding p53, and a cancer comprising cancer cells with a loss of function mutation in a gene encoding p53.

In some embodiments, the cancer is a hematological cancer or a lymphatic cancer.

In some embodiments, the cancer is a DNA damage repair pathway deficient cancer and/or a homologous-recombination deficient cancer.

In some embodiments, the cancer is a DNA damage repair pathway deficient cancer.

In some embodiments, the cancer is a homologous-recombination deficient cancer.

In some embodiments, the cancer is selected from the group consisting of a hematological cancer, a lymphatic cancer, a cancer comprising cancer cells with a mutation in a gene encoding p53, and a cancer comprising cancer cells with a loss of function mutation in a gene encoding p53.

In some embodiments, the cancer comprises cancer cells with a mutation in a gene encoding p53.

In some embodiments, the mutation in the gene encoding p53 is a loss of function mutation.

In some embodiments, the disclosure comprises administering to the subject in need thereof a therapeutically effective amount of a compound of the present disclosure along with one or more additional chemotherapeutic agents independently selected from anti-proliferative agents, anti-cancer agents, immunosuppressant agents and pain-relieving agents.

Methods of treatment according to the present disclosure general include administering a safe and effective amount of a compound according to formula (I) or a pharmaceutically acceptable salt thereof to a patient (particularly a human) in need thereof.

Compounds of the disclosure are indicated both in the therapeutic and/or prophylactic treatment of the above-mentioned conditions. For the above-mentioned therapeutic uses the dosage administered will typically vary with the compound employed, the mode of administration, the treatment desired and the disorder or disease indicated.

The compounds of the present disclosure may be used as single drug or as a pharmaceutical composition in which the compound is mixed with various pharmacologically acceptable materials.

In some embodiments, the compounds of the present disclosure can also contain unnatural proportions of atomic isotopes at one or more of the atoms that constitute such compounds. For example, the present disclosure also embraces isotopically-labeled variants of the present disclosure which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the disclosure and their uses. Exemplary isotopes that can be incorporated in to compounds of the disclosure include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds of the present disclosure can generally be prepared by following procedures analogous to those disclosed in the schemes and/or in the examples herein below, by substituting an isotopically labeled reagent for a non-isotopically labeled reagent.

### EXPERIMENTAL SECTION

Also provided are methods for the preparation of compound of formula (I) according to the description provided herein using appropriate methods and/or materials. It is to be understood by those skilled in the art that known variations of the conditions and processes of the following procedures can be used to prepare these intermediates and compounds. Moreover, by utilizing the procedures described in detail, one of ordinary skill in the art can prepare additional compounds of the present disclosure.

Following general guidelines apply to all experimental procedures described here. Unless otherwise stated, experiments are performed under positive pressure of nitrogen, temperature described are the external temperature (oil bath temperature). Reagents and solvents received from vendors are used as such without any further drying or purification. Molarities mentioned here for reagents in solutions are approximate as it was not verified by a prior titration with a standard. All reactions are stirred under magnetic stir bar. Cooling to minus temperature was done by acetone/dry ice or wet ice/salts. Magnesium sulfate and sodium sulfate were used as solvent drying agent after reaction work up and are interchangeable. Removing of solvents under reduced pressure or under vacuum means distilling of solvents in rotary evaporator.

Compounds of this disclosure may be made by synthetic chemical processes, examples of which are shown herein. It is meant to be understood that the order of the steps in the processes may be varied, that reagents, solvents and reaction conditions may be substituted for those specifically mentioned and that vulnerable moieties may be protected and deprotected, as necessary.

The specifics of the process for preparing compounds of the present disclosure are detailed in the experimental section.

The present disclosure shall be illustrated by means of some examples, which are not construed to be viewed as limiting the scope of the disclosure.

Unless otherwise stated, work-up includes distribution of the reaction mixture between the organic and aqueous phases, separation of layers and drying the organic layer over anhydrous sodium sulphate, filtration and evaporation of the solvent. Purification, unless otherwise mentioned, includes purification by silica gel chromatographic techniques, generally using ethyl acetate/petroleum ether mixture of a suitable polarity as the mobile phase.

Analysis for the compounds of the present disclosure unless mentioned, was conducted in general methods well known to a person skilled in the art. Having described the disclosure with reference to certain preferred embodiments, other embodiments will become apparent to one skilled in the art from consideration of the specification. The disclosure is further defined by reference to the following examples, describing in detail the analysis of the compounds of the disclosure.

It will be apparent to those skilled in the art that many modifications, both to materials and methods, may be practiced without departing from the scope of the disclosure. Some of the intermediates were taken to next step based on TLC results, without further characterization, unless otherwise specified.

### Abbreviations

| Abbreviation | Name |
|---|---|
| DMF-N,N- | N,N-Dimethylformamide; |
| Dimethylformamide | |
| ACN | Acetonitrile |
| EtOAc | Ethyl acetate |
| THF | Tetrahydrofuran |
| DCM | Dichloromethane |
| MeOH | Methanol |
| EtOH | Ethanol |
| DMSO | Dimethyl solfoxide |
| Mel | Methyl iodide |
| EtI | Ethyl iodide |
| TEA (or) Et₃N | Triethylamine |
| DIPEA | N,N-Diisopropylethylamine |
| K₂CO₃ | Potassium carbonate |
| NaOAc | Sodium acetate |
| KOAc | Potassium acetate |
| NH₄OH | Ammonium hydroxide |
| NH₄Cl | Ammonium chloride |
| BnBr | Benzyl bromide |
| h | Hour |
| i-PrI | Isopropyl iodide |
| DHP | 3,4-Dihydropyran |
| PTSA | p-Toluenesulfonic acid |
| mmol | Millimoles |
| mL | Milliliters |
| M | Molar |
| ° C. | Degree Celsius |
| Min | Minutes |
| equiv. | Equivalents |
| HATU | 1-[Bis(dimethylamino)methylene]-1H-1,2,3-triazolo[4,5- |
| | b]pyridinium 3-oxide hexafluorophosphate |
| RuPhos | 2-Dicyclohexylphosphino-2′,6′-diisopropoxybiphenyl |
| TFA | Trifluoroacetic acid |
| AcOH | Acetic acid |
| Pd(dppf)Cl₂ · DCM | [1,1′- |
| | Bis(diphenylphosphino)ferrocene]dichloropalladium(II), |
| | complex with dichloromethane |
| Pd(OAc)₂ | Palladium(II) acetate |
| Pd₂(dba)₃ | Tris(dibenzylideneacetone)dipalladium(0) |
| RT/rt | Room temperature |
| TLC | Thin layer chromatography |
| LCMS | Liquid chromatography-mass spectrometry |
| HPLC | High performance liquid chromatography |
| DMSO-d₆ | Deuterated dimethyl sulfoxide |
| Chloroform-d | Deuterated chloroform |
| NMR | Nuclear magnetic resonance |
| s | Singlet |
| d | Doublet |
| t | Triplet |
| q | quartet |
| app. quint. | Apparent quintet |
| dd | doublet of doublets |
| td | Triplet of doublets |
| SM | Starting material |
| Int | Intermediate |

### General Synthetic Schemes:

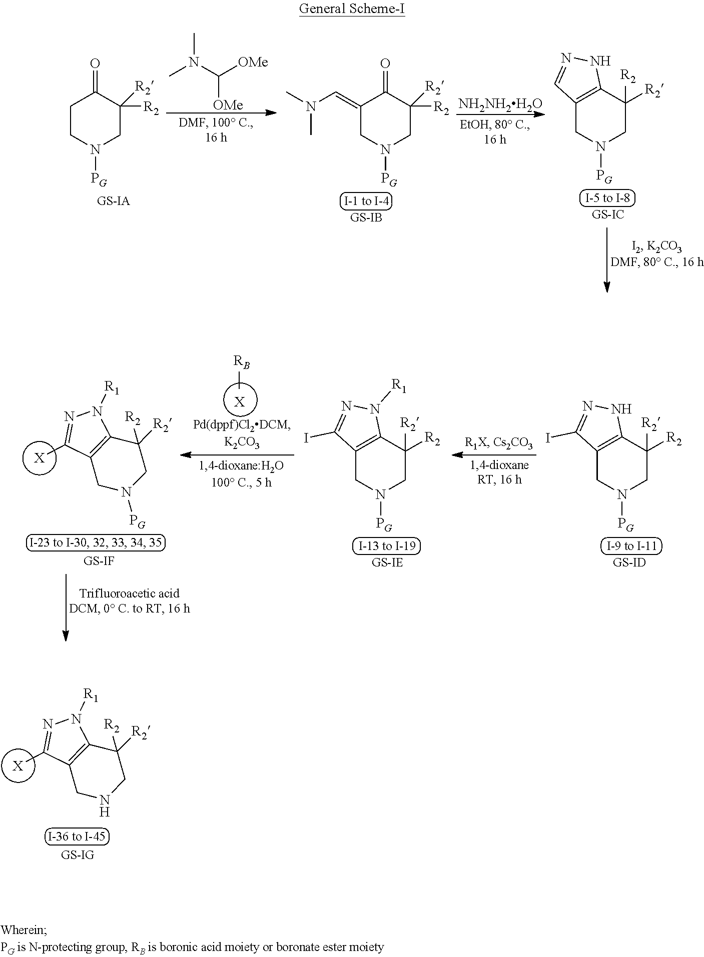

Some intermediates may be generally synthesized utilizing the process outlined in General Scheme-I. The commercially available or synthesized GS-IA was reacted with dimethylformamide dimethyl acetal in presence of suitable reagents and solvents (DMF, 100° C., 16 h) to obtain GS-IB which upon reacting with hydrazine hydrate in presence of suitable reagents and solvents (EtOH, 80° C., 16 h) afforded GS-IC. Treatment of GS-IC with iodine in presence of suitable reagents and solvent(s) (K₂CO₃, DMF, 80° C., 16 h) gave GS-ID. This GS-ID upon reacting with R₁-halide (R₁X) in presence of base such as cesium carbonate and solvent 1,4-dioxane affords GS-IE. This upon coupling with appropriate boronic acid or boronate ester in presence of suitable catalyst, base and solvent (Pd(dppf)Cl₂·DCM, K₂CO₃, 1,4-dioxane:H₂O, 100° C., 5 h) affords GS-IF. This GS-IF on deprotection using trifluoroacetic acid in appropriate solvent (DCM) affords intermediate GS-IG.

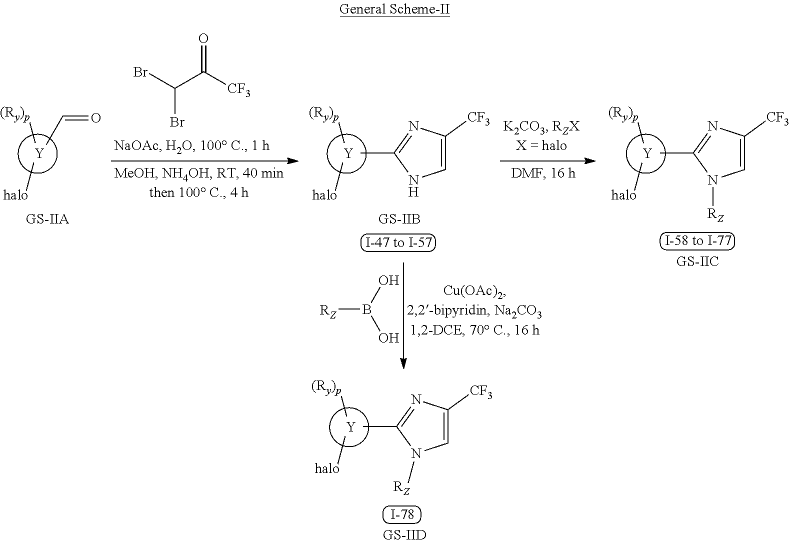

Some intermediates may be generally synthesized utilizing the process outlined in General Scheme-II. The commercially available or synthesized GS-IIA was reacted with 3,3-dibromo-1,1,1-trifluoropropan-2-one in presence of suitable reagents and solvents (NaOAc, H₂O, 100° C., 1 h; MeOH, NH₄OH, RT, 40 min then 100° C., 4 h) to obtain GS-LIB which upon reacting with R_{Z} halide in presence of suitable reagents and solvents (K₂CO₃, DMF) afforded GS-LIC. Alternatively, treatment of GS-IIB with appropriate RZ-boronic acid in presence of suitable reagents and solvent(s) (Cu(OAc)₂, O₂, 2,2′-bipyridin, Na₂CO₃, 1,2-DCE, 70° C., 16 h) gave GS-IID.

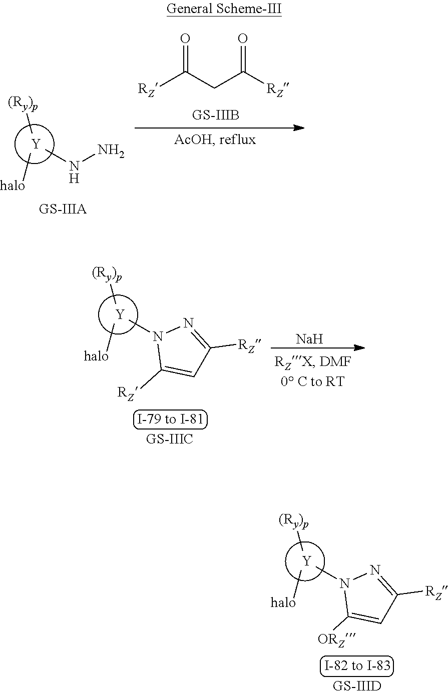

Some intermediates may be generally synthesized utilizing the process outlined in General Scheme-III. The commercially available or synthesized GS-IIIA was reacted with GS-IIIB in presence of suitable reagents and solvents (AcOH, reflux) to obtain GS-IIIC which upon reacting with R_{Z′″}-halide in presence of suitable reagents and solvents (NaH, DMF) afforded intermediate GS-IIID.

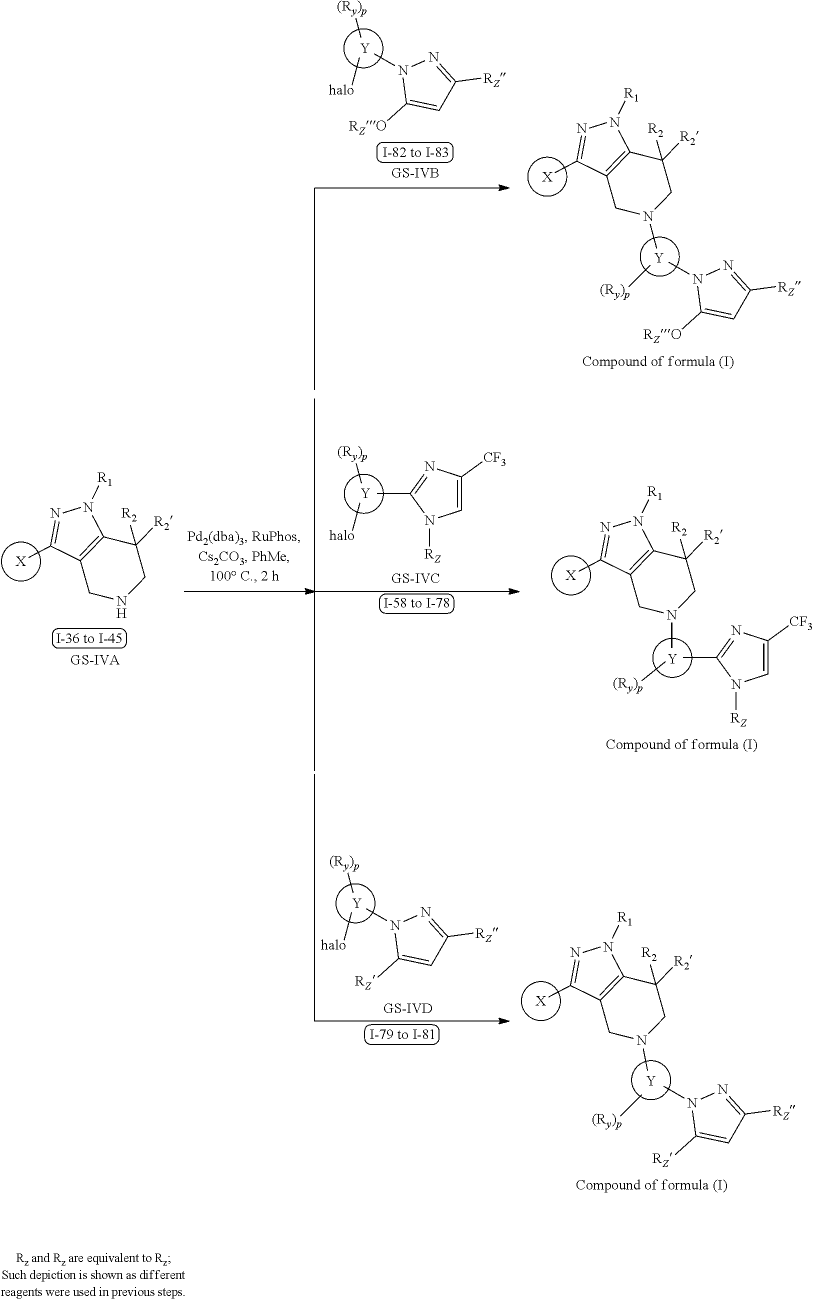

Some compounds of the present disclosure may be generally synthesized utilizing the process outlined in General Scheme-IV. The commercially available or synthesized GS-IVA was reacted with appropriate intermediates GS-IVB or GS-IVC or GS-IVD, in presence of suitable reagents and solvents (Pd₂(dba)₃, RuPhos, Cs₂CO₃, PhMe, 100° C., 2 h) afford respective compound of formula (I).

### Synthesis of Intermediates

### Synthesis of tert-butyl (E)-3-((dimethylamino)methylene)-4-oxopiperidine-1-carboxylate (I-1)

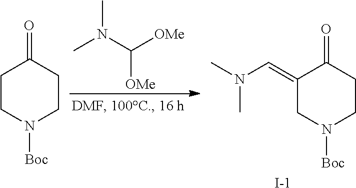

A mixture of tert-butyl 4-oxopiperidine-1-carboxylate (100.0 g, 501.88 mmol, 1.0 equiv.) and dimethylformamide dimethyl acetal (72.37 g, 602.25 mmol, 1.3 equiv.) in DMF (400 mL) was stirred at 100° C. for 16 hours. After that, the reaction mixture was cooled to room temperature and concentrated under reduced pressure to give crude enamine I-1 (120.0 g) as a brown oil, which was subjected to the next step without further purification.

¹H-NMR (400 MHz, DMSO-d₆): δ 7.30 (s, 1H), 4.47 (s, 2H), 3.47 (t, J=5.6 Hz, 2H), 3.07 (s, 6H), 2.26 (t, J=5.6 Hz, 2H), 1.40 (s, 9H).

The intermediates in Table 1 were prepared according to the procedure described in I-1 and using the appropriate starting material as given here.

TABLE 1

| Int. No. | SM | Int. Structure | Analytical Data |
|---|---|---|---|
| I-2 | 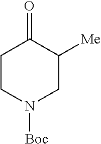 | 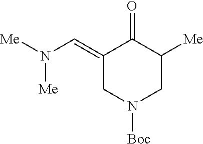 [SMILES: CC1CN(C(=O)OC(C)(C)C)C/C(=C\N(C)C)C1=O] | LC-MS: m/z 269.1 (M + H)⁺ |
| I-3 | 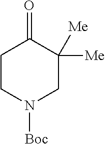 | 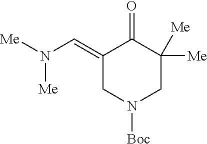 [SMILES: CN(C)/C=C1\CN(C(=O)OC(C)(C)C)CC(C)(C)C1=O] | ¹H NMR (400 MHZ, DMSO-d₆): δ 7.27 (s, IH), 4.52 (s, 2H), 3.32 (s, 2H), 3.05 (s, 6H), 1.41 (s, 9H), 0.95 (s, 6H) |
| I-4 | 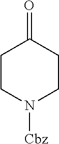 | 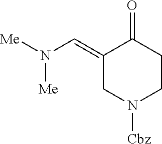 [SMILES: CN(C)/C=C1\CN(C(=O)OCc2ccccc2)CCC1=O] | LC-MS: m/z 289.2 (M + H)⁺ |

### Synthesis of tert-butyl 1,4,6,7-tetrahydro-5H-pyrazolo[4,3-c]pyridine-5-carboxylate (I-5)

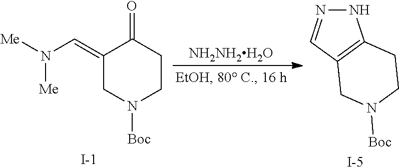

To a stirred solution of intermediate I-1 (6.0 g, 23.59 mmol, 1.0 equiv.) in ethanol (60 mL) was added hydrazine hydrate (2.36 g, 50.06 mmol, 2.0 equiv.) and the mixture was stirred at 80° C. for 16 hours. The mixture was concentrated under reduced pressure to give crude product, which was purified by combi-flash column chromatography using ethyl acetate in hexane to afford the pure product pyrazole I-5 (3.0 g) as an off-white solid.

¹H NMR (400 MHz, DMSO-d₆): δ 12.50 (bs, 1H), 7.49 (s, 0.6H)*, 7.27 (s, 0.4H)*, 4.40-4.32 (m, 2H), 3.58 (t, J=5.6 Hz, 2H), 2.67-2.58 (m, 2H), 1.41 (s, 9H); LC-MS: m/z 224.1 (M+H)⁺* This hydrogen was split into two peaks because of 3:2 ratio of pyrazole tautomers.

The intermediates in Table 2 were prepared according to the procedure described in I-5, using the appropriate starting material as given here.

TABLE 2

| Int. No. | SM | Int. Structure | Analytical Data |
|---|---|---|---|
| I-6 | I-2 | 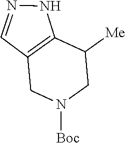 [SMILES: CC1CN(C(=O)OC(C)(C)C)Cc2cn[nH]c21] | LC-MS: 238.1 m/z (M + H)⁺ |
| I-7 | I-3 | 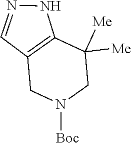 [SMILES: CC(C)(C)OC(=O)N1Cc2cn[nH]c2C(C)(C)C1] | LC-MS: 252.1 m/z (M + H)⁺ |
| I-8 | I-4 | 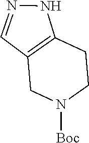 [SMILES: CC(C)(C)OC(=O)N1CCc2[nH]ncc2C1] | LC-MS: 258.0 m/z (M + H)⁺ |

### Synthesis of tert-butyl 3-iodo-1,4,6,7-tetrahydro-5H-pyrazolo[4,3-c]pyridine-5-carboxylate (I-9)

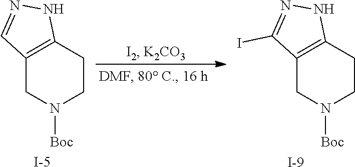

To a mixture of intermediate I-5 (5.0 g, 22.39 mmol, 1.0 equiv.) and potassium carbonate (9.28 g, 67.17 mmol, 3.0 equiv.) in DMF (30 mL) was added iodine (22.73 g, 89.57 mmol, 4.0 equiv.). The mixture was heated to 80° C. and stirred for 16 hours. The reaction mixture was quenched with aq. sodium thiosulphate and extracted with ethyl acetate (3×). The combined organic layers were washed with brine and dried over anhydrous sodium sulphate. The filtered organic layer was concentrated to give crude product. The crude material was washed with diethyl ether and hexane to give the iodopyrazole I-9 (4.2 g) as an off-white solid.

¹H NMR (400 MHz, DMSO-d₆): δ 13.0 (bs, 1H), 4.13 (s, 2H), 3.59 (t, J=5.8 Hz, 2H), 2.63 (t, J=5.6 Hz, 2H), 1.42 (s, 9H); LC-MS: m/z 350.0 (M+H)⁺

The intermediates in Table 3 were prepared according to the procedure described in I-9, using the appropriate starting material as given here.

TABLE 3

| Int. No. | SM | Int. Structure | Analytical Data |
|---|---|---|---|
| I-10 | I-6 | 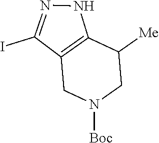 [SMILES: CC1CN(C(=O)OC(C)(C)C)Cc2c(I)n[nH]c21] | LC-MS: 363.9 m/z (M + H)' |
| I-11 | I-7 | 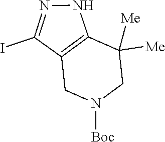 [SMILES: CC(C)(C)OC(=O)N1Cc2c(I)n[nH]c2C(C)(C)C1] | LC-MS: 377.9 m/z (M + H)⁺ |
| I-12 | I-8 | 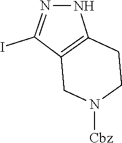 [SMILES: O=C(OCc1ccccc1)N1CCc2[nH]nc(I)c2C1] | LC-MS: 384.1 m/z (M + H)⁺ |

### Synthesis of tert-butyl 3-iodo-1-methyl-1,4,6,7-tetrahydro-5H-pyrazolo[4,3-c]pyridine-5-carboxylate (1-13)

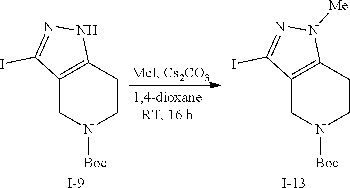

To a stirred solution of intermediate I-9 (18.6 g, 53.27 mmol, 1.0 equiv.) in 1,4-dioxane (100 mL) was added cesium carbonate (52.07 g, 159.8 mmol, 3.0 equiv.) followed by methyl iodide (15.12 g, 106.53 mmol, 2.0 equiv.) at room temperature and the mixture was stirred for 16 hours. The reaction mixture was diluted with ice-cold water and then extracted with ethyl acetate (3×). The combined organic layers were washed with brine and dried over anhydrous sodium sulphate. The filtered organic layer was concentrated to give crude product. The crude material was washed with hexane to give the methylated product 1-13 (12.0 g) as an off-white solid.

¹H NMR (400 MHz, DMSO-d₆): δ 4.10 (s, 2H), 3.70 (s, 3H), 3.60 (t, J=5.8 Hz, 2H), 2.65 (t, J=5.6 Hz, 2H), 1.42 (s, 9H); LC-MS: m/z 364.0 (M+H)⁺.

The intermediates in Table 4 were prepared according to the procedure described in I-13, using the appropriate starting materials as given here.

TABLE 4

| Int. No. | SM-1 | SM-2 | Int. Structure | Analytical Data |
|---|---|---|---|---|
| I-14 | I-9 | EtI | 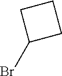 | ¹H NMR (400 MHZ, DMSO-d₆): δ 4.10 (s, 2H), 4.01 (q, J = 7.2 Hz, 2H), 3.61 (t, J = 5.8 Hz, 2H), 2.67 (t, J = 5.6 Hz, 2H), 1.42 (s, 9H), 1.28 (t, J = 7.2 Hz, 3H) |
| I-15 | I-9 | i-PrI | 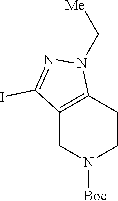 [SMILES: CCn1nc(I)c2c1CCN(C(=O)OC(C)(C)C)C2] | ¹H NMR (400 MHZ, DMSO-d₆): δ 4.42-4.35 (m, 1H), 4.14 (s, 2H), 3.56 (t, J = 5.6 Hz, 2H), 2.64 (1, J = 5.6 Hz, 2H), 1.41 (s, 9H), 1.30 (d, J = 6.8 Hz, 6H) |
| I-16 | I-9 | Br | 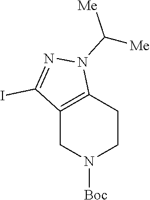 [SMILES: CC(C)n1nc(I)c2c1CCN(C(=O)OC(C)(C)C)C2] | ¹H NMR (400 MHZ, DMSO-d₆): δ 4.75-4.66 (m, IH), 4.16 (s, 2H), 3.58 (t, J = 5.6 Hz, 2H), 2.64 (t, J = 5.6 Hz, 2H), 2.50-2.49 (m, 2H), 2.45-2.38 (m, 2H), 1.82-1.72 (m, 2H), 1.42 (s, 9H) |
| I-17 | I-9 | BnBr | 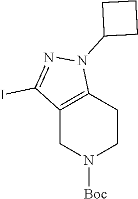 [SMILES: CC(C)(C)OC(=O)N1CCc2c(c(I)nn2C2CCC2)C1] | LC-MS: m/z 440.0 (M + H)⁺ |
| I-18 | I-10 | Mel | 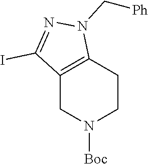 [SMILES: CC(C)(C)OC(=O)N1CCc2c(c(I)nn2Cc2ccccc2)C1] | LC-MS: 377.9 m/z (M + H)⁺ |
| I-19 | I-11 | Mel | 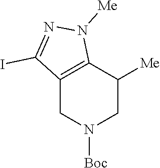 [SMILES: CC1CN(C(=O)OC(C)(C)C)Cc2c(I)nn(C)c21] | LC-MS: m/z 392.0 (M + H)⁺ |

### Synthesis of benzyl 3-iodo-1-(tetrahydro-2H-pyran-2-yl)-1,4,6,7-tetrahydro-5H-pyrazolo[4,3-c]pyridine-5-carboxylate (I-20)

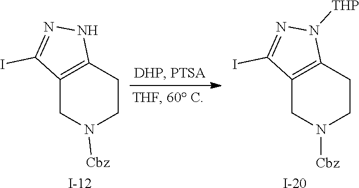

To a stirred solution of intermediate I-12 (0.3 g, 0.783 mmol, 1.0 equiv.) in THF (5 mL) were added 3,4-dihydro-2H-pyran (0.099 g, 1.175 mmol, 1.5 equiv.) and PTSA (0.074 g, 0.390 mmol, 0.5 equiv.) at room temperature. The reaction mixture was heated to 60° C. for 6 hours. The reaction mixture was cooled to room temperature, diluted with ice-cold water, and extracted with ethyl acetate (3×). The combined organic layers were washed with brine and dried over anhydrous sodium sulphate. The filtered organic layer was concentrated to give crude product, which was purified by combi-flash column chromatography using ethyl acetate in hexane to afford the pure product pyrazole I-20 (0.28 g).

LC-MS: 468.0 m/z (M+H)⁺

### Synthesis of tert-butyl 1-cyclopropyl-3-iodo-1,4,6,7-tetrahydro-5H-pyrazolo[4,3-c]pyridine-5-carboxylate (I-21)

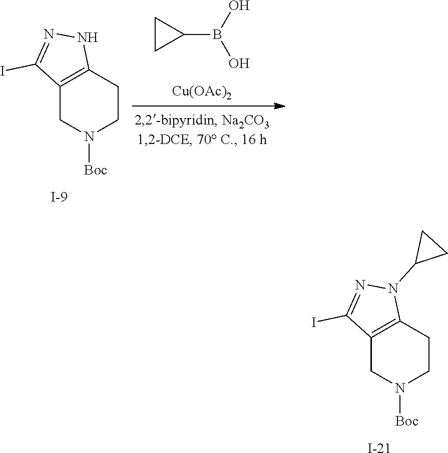

A mixture of intermediate I-9 (0.1 g, 0.29 mmol, 1.0 equiv.), cyclopropylboronic acid (0.049 g, 0.57 mmol, 2.0 equiv., commercial source) and sodium carbonate (0.061 g, 0.57 mmol, 2.0 equiv.) in 1,2-dichloroethane (5 mL) was heated to 70° C. At the same temperature were added copper(II) acetate (0.057 g, 0.28 mmol, 1.0 equiv.) and 2,2′-bipyridine (0.045 g, 0.28 mmol, 1.0 equiv.), then stirring was continued at the same temperature for 16 hours. The reaction mixture was quenched with acetic acid (1 mL), then diluted with water and extracted with ethyl acetate (3×). The combined organic layers were washed with brine and dried over anhydrous sodium sulphate. The filtered organic layer was concentrated to give crude product which was purified by combi-flash column chromatography using ethyl acetate in hexane to afford the pure coupled product I-21 (0.025 g) as an off-white solid.

¹H NMR (400 MHz, DMSO-d₆): δ 4.08 (s, 2H), 3.59 (t, J=5.6 Hz, 2H), 3.50-3.42 (m, 1H), 2.72 (t, J=5.6 Hz, 2H), 1.41 (s, 9H), 1.03-0.98 (m, 2H), 0.95-0.91 (m, 2H); LC-MS: m/z 389.9 (M+H)⁺

### Synthesis of 1-isopropyl-4-methyl-5-(4,4,5,5-tetramethyl-1,3,2-dioxaborolan-2-yl)-1H-pyrazole (1-22)

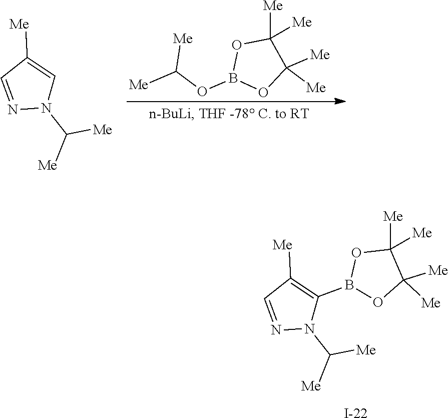

To a stirred solution of 1-isopropyl-4-methyl-1H-pyrazole (1.0 g, 8.05 mmol, 1.0 equiv.) in tetrahydrofuran (40 mL) was added n-butyl lithium (1.6M in hexane. 30 mL, 40.25 mmol, 5.0 equiv.) dropwise at 0° C. and the mixture was stirred for 1 hour at the same temperature. The reaction mixture was cooled to −78° C. 2-Isopropoxy-4,4,5,5-tetramethyl-1,3,2-dioxaborolane (3.75 g, 20.13 mmol, 2.50 equiv.) was added and the reaction mixture was allowed to warm to room temperature. The progress of the reaction was monitored by TLC. After completion of the reaction was confirmed by TLC, the reaction was quenched with ice-cold water, then extracted with ethyl acetate (3×). The combined organic layers were washed with brine and dried over anhydrous sodium sulphate. The filtered organic layer was concentrated to give crude product which was purified by combi-flash column chromatography using ethyl acetate in hexane to afford the pure product 1-22 (0.62 g).

¹H NMR (400 MHz, DMSO-d₆): 7.28 (s, 1H), 4.97 (septet, J=6.8 Hz, 1H), 2.13 (s, 3H), 1.35 (d, J=6.8 Hz, 6H), 1.30 (s, 12H).

### Synthesis of tert-butyl 3-(4-cyclopropyl-6-methoxypyrimidin-5-yl)-1-methyl-1,4,6,7-tetrahydro-5H-pyrazolo[4,3-c]pyridine-5-carboxylate (1-23)

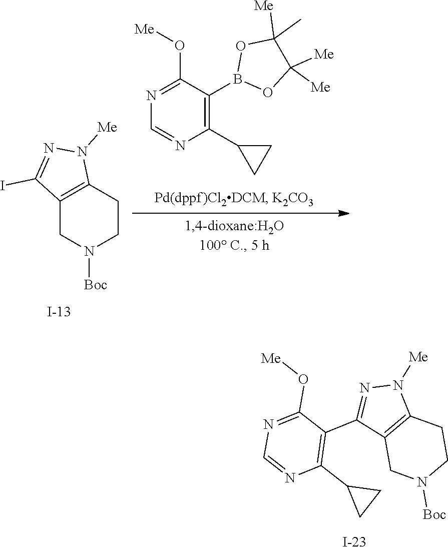

A mixture of intermediate I-13 (0.4 g, 1.10 mmol, 1.0 equiv.), 4-cyclopropyl-6-methoxy-5-(4,4,5,5-tetramethyl-1,3,2-dioxaborolan-2-yl)pyrimidine (0.456 g, 1.65 mmol, 1.5 equiv., commercial source) and potassium carbonate (0.46 g, 3.30 mmol, 3.0 equiv.) in 1,4-dioxane (15 mL) and water (5 mL) was degassed with argon for 10 min. After that, Pd(dppf)Cl₂·DCM (0.09 g, 0.11 mmol, 0.1 equiv.) was added and the reaction mixture was heated to 100° C. for 5 hours. The reaction mixture was cooled to room temperature, quenched with ice-cold water, and extracted with ethyl acetate (3×). The combined organic layers were washed with brine and dried over anhydrous sodium sulphate. The filtered organic layer was concentrated to give crude product which was purified by combi-flash column chromatography using ethyl acetate in hexane as an eluent to afford the coupled product I-23 (0.35 g) as a brown solid.

¹H NMR (400 MHz, DMSO-d₆): δ 8.60 (s, 1H), 4.15 (s, 2H), 3.87 (s, 3H), 3.69 (s, 3H), 3.63 (t, J=5.8 Hz, 2H), 2.73 (t, J=5.6 Hz, 2H), 2.19-2.05 (m, 1H), 1.39 (s, 9H), 1.05-1.00 (in, 2H), 0.90-0.85 (m, 2H); LC-MS: m/z 386.2 (M+H)⁺

The intermediates shown below in Table 5 were prepared according to a procedure similar to the one described in I-23 and using the appropriate starting materials SM1 and SM2 as shown in the table.

TABLE 5

| Int. No. | SM1 | SM2 | Int. Structure | Analytical Data |
|---|---|---|---|---|
| I-24 | I-14 | 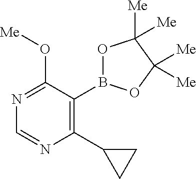 | 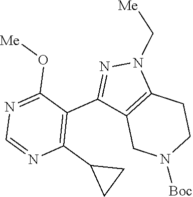 [SMILES: CCn1nc(-c2c(OC)ncnc2C2CC2)c2c1CCN(C(=O)OC(C)(C)C)C2] | ¹H NMR (400 MHZ, DMSO-d₆): δ 8.60 (s, 1H), 4.16 (s, 2H), 4.07 (q, J = 7.2 Hz, 2H), 3.90 (s, 3H), 3.64 (t, J = 5.8 Hz, 2H), 2.75 (t, J = 5.6 Hz, 2H), 2.22-2.15 (m, 1H), 1.42 (s, 9H), 1.32 (t, J = 7.2 Hz, 3H), 1.06-1.01 (m, 2H), 0.96-0.91 (m, 2H) |
| I-25 | I-15 | 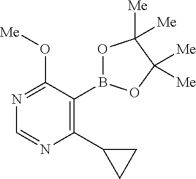 | 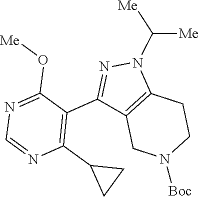 [SMILES: COc1ncnc(C2CC2)c1-c1nn(C(C)C)c2c1CN(C(=O)OC(C)(C)C)CC2] | ¹H NMR (400 MHZ, DMSO-d₆): δ 8.60 (s, 1H), 4.51-4.45 (m, 1H), 4.17 (s, 2H), 3.92 (s, 3H), 3.65 (t, J = 5.8 Hz, 2H), 2.76 (t, J = 5.6 Hz, 2H), 2.22-2.15 (m, 1H), 1.42 (s, 9H), 1.40 (d, J = 6.4 Hz, 6H), 1.06-1.01 (m, 2H), 0.96-0.91 (m, 2H) |
| I-26 | I-21 | 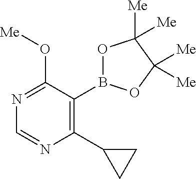 | 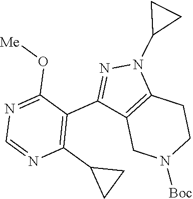 [SMILES: COc1ncnc(C2CC2)c1-c1nn(C2CC2)c2c1CN(C(=O)OC(C)(C)C)CC2] | ¹H NMR (400 MHZ, DMSO-d₆): δ 8.56 (s, 1H), 4.11 (s, 2H), 3.85 (s, 3H), 3.63 (t, J = 5.8 Hz, 2H), 3.52-3.47 (m, 1H), 2.80 (t, J = 5.6 Hz, 2H), 2.15-2.09 (m, 1H), 1.42 (s, 9H), 1.05-0.85 (m, 8H); LC-MS: m/z 412.3 (M + H)⁺ |
| I-27 | I-16 | 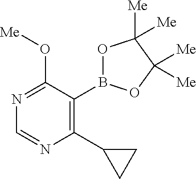 | 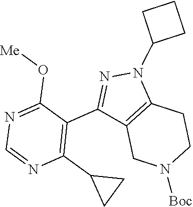 [SMILES: COc1ncnc(C2CC2)c1-c1nn(C2CCC2)c2c1CN(C(=O)OC(C)(C)C)CC2] | ¹H NMR (400 MHZ, DMSO-d₆): δ 8.61 (s, 1H), 4.79-4.72 (m, 1H), 4.14 (s, 2H), 3.93 (s, 3H), 3.63 (t, J = 5.8 Hz, 2H), 2.73 (t, J = 5.6 Hz, 2H), 2.55-2.50 (m, 2H), 2.38-2.33 (m, 2H), 2.20-2.14 (m, 1H), 1.82-1.72 (m, 2H), 1.41 (s, 9H), 1.05-0.1.01 (m, 2H), 0.98-0.90 (m, 2H); LC-MS: m/z 426.3 (M + H)⁺ |
| I-28 | I-17 | 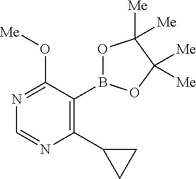 | 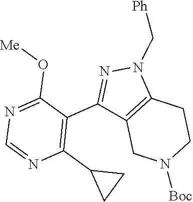 [SMILES: COc1ncnc(C2CC2)c1-c1nn(Cc2ccccc2)c2c1CN(C(=O)OC(C)(C)C)CC2] | ¹H NMR (400 MHZ, DMSO-d₆): δ 8.61 (s, 1H), 7.38-7.20 (m, 5H), 5.30 (s, 2H), 4.16 (s, 2H), 3.89 (s, 3H), 3.63 (t, J = 5.8 Hz, 2H), 2.69 (t, J = 5.6 Hz, 2H), 2.20- 2.14 (m, 1H), 1.42 (s, 9H), 1.05-0.1.01 (m, 2H), 0.95-0.89 (m, 2H); LC-MS: m/z 462.2 (M + H)⁺ |
| I-29 | I-18 | 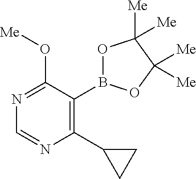 | 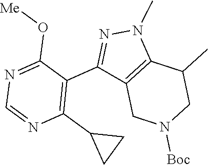 [SMILES: COc1ncnc(C2CC2)c1-c1nn(C)c2c1CN(C(=O)OC(C)(C)C)CC2C] | LC-MS: m/z 400.1 (M + H)⁺ |
| I-30 | I-19 | 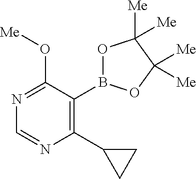 | 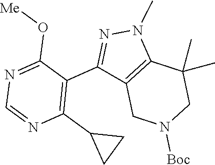 [SMILES: COc1ncnc(C2CC2)c1-c1nn(C)c2c1CN(C(=O)OC(C)(C)C)CC2(C)C] | LC-MS: m/z 414.2 (M + H)⁺ |
| I-31 | I-20 | 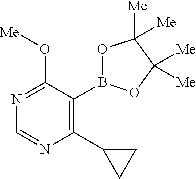 | 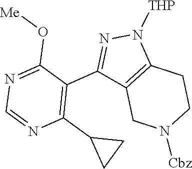 | LC-MS: m/z 490.3 (M + H)⁺ |
| I-32 | I-13 | 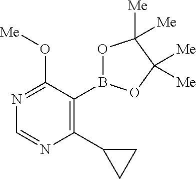 | 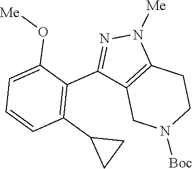 [SMILES: COc1cccc(C2CC2)c1-c1nn(C)c2c1CN(C(=O)OC(C)(C)C)CC2] | LC-MS: m/z 384.2 (M + H)⁺ |
| I-33 | I-13 | 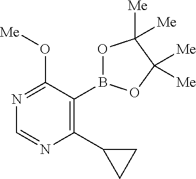 | 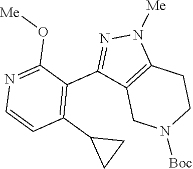 [SMILES: COc1nccc(C2CC2)c1-c1nn(C)c2c1CN(C(=O)OC(C)(C)C)CC2] | LC-MS: m/z 385.3 (M + H)⁺ |
| I-34 | I-13 | 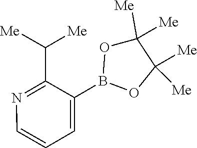 | 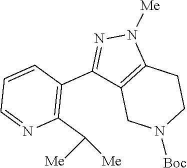 [SMILES: CC(C)c1ncccc1-c1nn(C)c2c1CN(C(=O)OC(C)(C)C)CC2] | LC-MS: m/z 357.3 (M + H)⁺ |
| I-35 | I-13 | 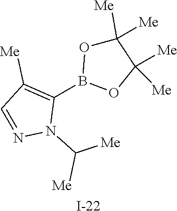 | 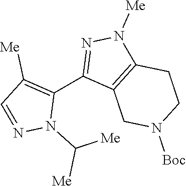 [SMILES: Cc1cnn(C(C)C)c1-c1nn(C)c2c1CN(C(=O)OC(C)(C)C)CC2] | LC-MS: m/z 360.2 (M + H)⁺ |

### Synthesis of 3-(4-cyclopropyl-6-methoxypyrimidin-5-yl)-1-methyl-4,5,6,7-tetrahydro-IH-pyrazolo[4,3-c]pyridine (1-36)

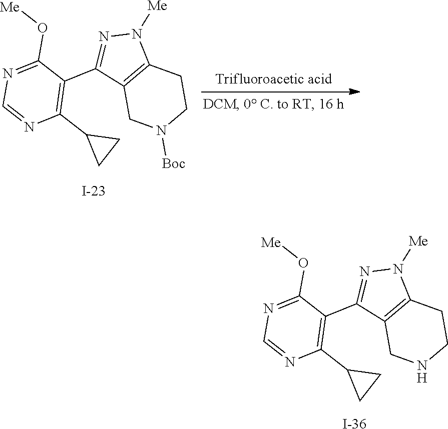

To a stirred solution of intermediate 1-23 (0.4 g, 1.04 mmol, 1.0 equiv.) in dichloromethane (10 mL) was added trifluoroacetic acid (0.35 g, 3.11 mmol, 3.0 equiv.) at 0° C. The reaction mixture was allowed to warm to room temperature and stirred for 16 hours. After that, the reaction mixture was concentrated under reduced pressure, quenched with sat. sodium bicarbonate solution and extracted with EtOAc (3×). The combined organic layers were washed with brine and dried over anhydrous sodium sulphate. The filtered organic layer was concentrated to give crude product 1-36 (0.15 g) as a brown oil, which was taken to the next step without further purification.

¹H NMR (400 MHz, DMSO-d₆): δ 8.57 (s, 1H), 3.86 (s, 3H), 3.72 (s, 3H), 3.44 (s, 2H), 2.94 (t, J=5.6 Hz, 2H), 261 (t, J=5.6 Hz, 2H), 2.18-2.12 (m, 1H), 1.05-1.00 (m, 2H), 0.95-0.91 (m, 2); LC-MS: m/z 286.1 (M+H)⁺

The intermediates in Table 6 were prepared according to the procedure described in I-36, using the appropriate starting material as given here.

TABLE 6

| Int. No. | SM | Int. Structure | Analytical Data |
|---|---|---|---|
| I-37 | I-24 | 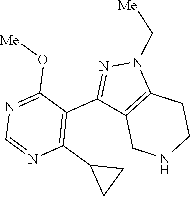 [SMILES: CCn1nc(-c2c(OC)ncnc2C2CC2)c2c1CCNC2] | 1H NMR (400 MHz, DMSO-d6): δ 8.59 (s, 1H), 4.05 (q, J = 6.8 Hz, 2H), 3.87 (s, 3H), 3.52 (s, 2H), 3.03 (t, J = 5.6 Hz, 2H), 2.67 (t, J = 5.6 Hz, 2H), 2.23-2.18 (m, 1H), 1.33 (t, J = 6.8 Hz, 3H), 1.05-1.00 (m, 2H), 0.95-0.89 (m, 2H); LC-MS: m/z 300.1 (M + H)+ |
| I-38 | I-25 | 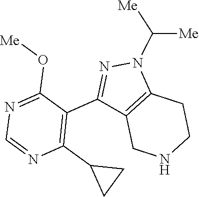 [SMILES: COc1ncnc(C2CC2)c1-c1nn(C(C)C)c2c1CNCC2] | 1H NMR (400 MHz, DMSO-d6): δ 8.56 (s, 1H), 4.48-4.42 (m, 1H), 3.87 (s, 3H), 3.43 (s, 2H), 2.94 (t, J = 5.6 Hz, 2H), 2.62 (t, J = 5.6 Hz, 2H), 2.23-2.18 (m, 1H), 1.38 (d, J = 6.8 Hz, 6H), 1.05-0.99 (m, 2H), 0.92-0.85 (m, 2H); LC-MS: m/z 314.1 (M + H)+ |
| I-39 | I-26 | 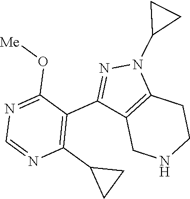 [SMILES: COc1ncnc(C2CC2)c1-c1nn(C2CC2)c2c1CNCC2] | 1H NMR (400 MHz, DMSO-d6): δ 8.57 (s, 1H), 3.86 (s, 3H), 3.53-3.49 (m, 1H), 3.43 (s, 2H), 2.94 (t, J = 5.6 Hz, 2H), 2.71 (t, J = 5.6 Hz, 2H), 2.15-2.11 (m, 1H), 1.05-0.88 (m, 8H); LC-MS: m/z 312.2 (M + H)+ |
| I-40 | I-27 | 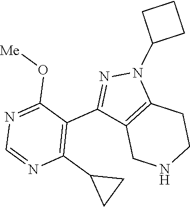 [SMILES: COc1ncnc(C2CC2)c1-c1nn(C2CCC2)c2c1CNCC2] | 1H NMR (400 MHz, DMSO-d6): δ 8.55 (s, 1H), 4.75-4.68 (m, 1H), 3.83 (s, 3H), 3.40 (s, 2H), 2.90 (t, J = 5.6 Hz, 2H), 2.57 (t, J = 5.6 Hz, 2H), 2.51-2.48 (m, 2H), 2.35-2.46 (m, 2H), 2.18-2.11 (m, 1H), 1.79-1.60 (m, 2H), 0.99- 0.95 (m, 2H), 0.91-0.86 (m, 2H); LC-MS: m/z 326.2 (M + H)+ |
| I-41 | I-28 | 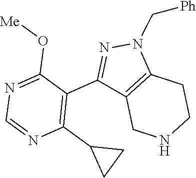 [SMILES: COc1ncnc(C2CC2)c1-c1nn(Cc2ccccc2)c2c1CNCC2] | 1H NMR (400 MHz, DMSO-d6): δ 8.59 (s, 1H), 7.39-7.20 (m, 5H), 5.29 (s, 2H), 3.88 (s, 3H), 3.45 (s, 2H), 2.92 (t, J = 5.6 Hz, 2H), 2.56 (t, J = 5.6 Hz, 2H), 2.18-2.11 (m, 1H), 1.04- 0.99 (m, 2H), 0.92-0.85 (m, 2H); LC-MS: m/z 362.1 (M + H)+ |
| I-42 | I-29 | 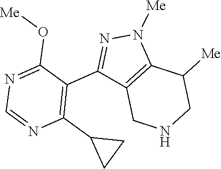 [SMILES: COc1ncnc(C2CC2)c1-c1nn(C)c2c1CNCC2C] | LC-MS: m/z 300.1 (M + H)+ |
| I-43 | I-30 | 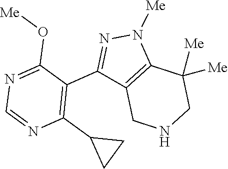 [SMILES: COc1ncnc(C2CC2)c1-c1nn(C)c2c1CNCC2(C)C] | LC-MS: m/z 314.1 (M + H)+ |
| I-44 | I-32 | 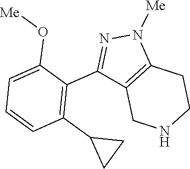 [SMILES: COc1cccc(C2CC2)c1-c1nn(C)c2c1CNCC2] | LC-MS: m/z 284.2 (M + H)+ |
| I-45 | I-34 | 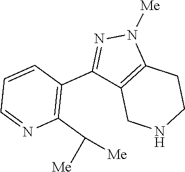 [SMILES: CC(C)c1ncccc1-c1nn(C)c2c1CNCC2] | LC-MS: m/z 257.1 (M + H)+ |

### Synthesis of 3-(4-cyclopropyl-6-methoxypyrimidin-5-yl)-1-(tetrahydro-2H-pyran-2-yl)-4,5,6,7-tetrahydro-1H-pyrazolo[4,3-c]pyridine (I-46)

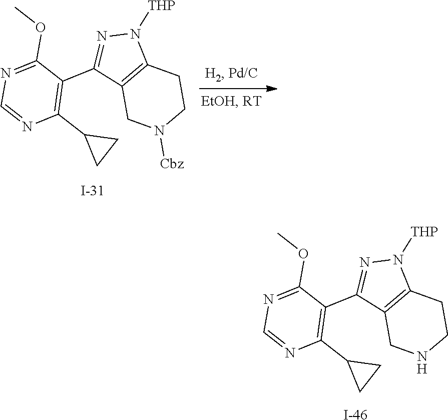

A mixture of intermediate I-31 (0.20 g, 0.409 mmol, 1.0 equiv.) and 10% palladium on charcoal (0.22 g, 2.04 mmol, 5.0 equiv.) in ethanol (10 mL) was stirred under an atmosphere of hydrogen for 4 hours at room temperature. The reaction mixture was filtered through a pad of Celite® and then the filtrate was concentrated under reduced pressure to provide crude product I-46 (0.14 g), which was subjected to the next step without further purification.

LC-MS: m/z 356.2 (M+H)⁺

### Synthesis of 2-(4-bromophenyl)-4-(trifluoromethyl)-1H-imidazole (I-47)

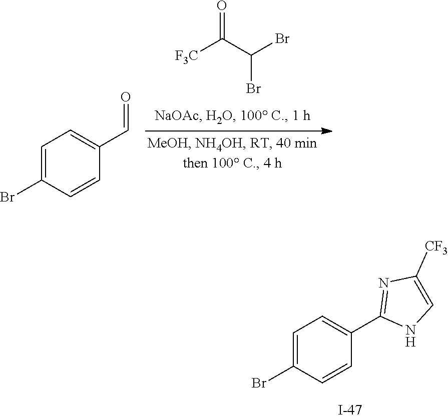 [SMILES: O=C(C(Br)Br)C(F)(F)F ; O=Cc1ccc(Br)cc1 ; FC(F)(F)c1c[nH]c(-c2ccc(Br)cc2)n1]

To a stirred solution of 4-bromobenzaldehyde (14.58 g, 54.05 mmol, 1.0 equiv.) in water (30 mL) was added NaOAc (6.65 g, 81.07 mmol, 1.5 equiv.) and the mixture was stirred at 100° C. for 1 hour. The reaction mixture was then cooled to 0° C. A pre-mixed solution of 3,3-dibromo-1,1,1-trifluoropropan-2-one (10.0 g, 54.05 mmol, 1.0 equiv.) and aq. ammonia (25% in water, 35 mL) in MeOH (150 mL) was added at 0° C. to the reaction mixture and the mixture was stirred at room temperature for 40 min. After that, the reaction mixture was heated to 100° C. under stirring for 4 hours. The reaction mixture was quenched with ice-cold water and then extracted with ethyl acetate (3×). The combined organic layers were washed with brine and dried over anhydrous sodium sulphate. The filtered organic layer was concentrated to give crude product which was purified by combi-flash column chromatography using ethyl acetate in hexane to afford the pure imidazole product I-47 (6 g) as a brown solid.

¹H NMR (400 MHz, DMSO-d₆): δ 13.29 (bs, 1H), 7.96 (s, 1H), 7.94 (d, J=8.4 Hz, 2H), 769 (d, J=8.4 Hz, 2H)

The intermediates in Table 7 were prepared according to the procedure described in I-47, using the appropriate starting material as given here.

TABLE 7

| Int. No. | SM | Int. Structure | Analytical Data |
|---|---|---|---|
| I-48 | 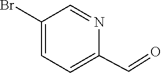 | 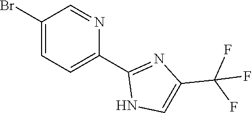 [SMILES: FC(F)(F)c1c[nH]c(-c2ccc(Br)cn2)n1] | 1H NMR (400 MHz, DMSO-d6): δ 13.58 (bs, 1H), 8.75 (d, J = 2.0 Hz, 1H), 8.15 (dd, J = 8.0, 2,0 Hz, 1H), 7.99 (d, J = 8.4 Hz, 1H), 7.87 (s, 1H); LC-MS: m/z 293.8 (M + H)+ |
| I-49 | 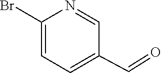 | 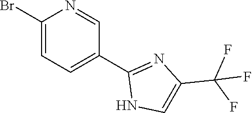 [SMILES: FC(F)(F)c1c[nH]c(-c2ccc(Br)nc2)n1] | 1H NMR (400 MHz, DMSO-d6): δ 13.49 (bs, 1H), 8.91 (d, J = 2.4 Hz, 1H), 8.22 (dd, J = 8.4, 2,4 Hz, 1H), 8.02 (s, 1H), 7.77 (d, J = 8.4 Hz, 1H); LC-MS: m/z 293.8 (M + H)+ |
| I-50 | 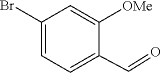 | 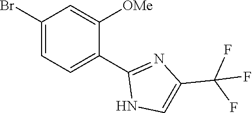 [SMILES: COc1cc(Br)ccc1-c1nc(C(F)(F)F)c[nH]1] | 1H NMR (400 MHz, DMSO-d6): δ 12.40 (bs, 1H), 7.97 (d, J = 8.4 Hz, 1H), 7.78 (s, 1H), 7.40 (d, J = 2.0 Hz, 1H), 7.26 (dd, J = 8.4, 2.0 Hz, 1H), 3.98 (s, 3H); LC-MS: m/z 322.9 (M + H)+ |
| I-51 | 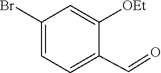 | 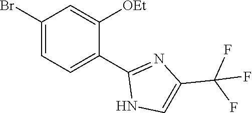 [SMILES: CCOc1cc(Br)ccc1-c1nc(C(F)(F)F)c[nH]1] | 1H NMR (400 MHz, DMSO-d6): δ 12.40 (bs, 1H), 7.90 (d, J = 8.4 Hz, 1H), 7.78 (s, 1H), 7.40 (d, J = 2.0 Hz, 1H), 7.25 (dd, J = 8.4, 1.6 Hz, 1H), 4.29 (q. J = 6.8 Hz, 2H), 1.40 (t. J = 6.8 Hz, 3H); LC-MS: m/z 334.9 (M + H)+ |
| I-52 | 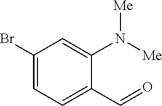 | 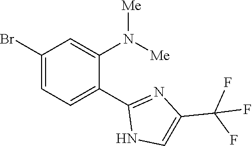 [SMILES: CN(C)c1cc(Br)ccc1-c1nc(C(F)(F)F)c[nH]1] | 1H NMR (400 MHz, DMSO-d6): δ 12.67 (bs, 1H), 7.77 (s, 1H), 7.72 (d, J = 8.0 Hz, 1H), 7.35 (d, J = 1.6 Hz, 1H), 7.27 (dd, J = 8.4, 2.0 Hz, 1H), 2.47 (s, 6H). |
| I-53 | 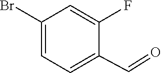 | 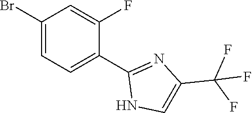 [SMILES: Fc1cc(Br)ccc1-c1nc(C(F)(F)F)c[nH]1] | 1H NMR (400 MHz, DMSO-d6): δ 13.00 (bs, 1H), 7.94 (s, 1H), 7.90 (d, J = 8.4 Hz, 1H), 7.77 (dd, J = 9.8, 1.6 Hz, 1H), 7.56 (d, J = 8.4, 2.0 Hz, 1H) |
| I-54 | 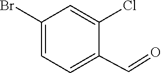 | 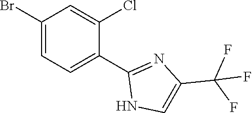 [SMILES: FC(F)(F)c1c[nH]c(-c2ccc(Br)cc2Cl)n1] | 1H NMR (400 MHz, DMSO-d6): δ 13.00 (bs, 1H), 7.97 (d, J = 0.8 Hz, 1H), 7.93 (s, 1H), 7.70 (s, 2H); LC-MS: m/z 326.9 (M + H)+. |
| I-55 | 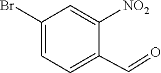 | 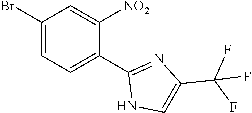 [SMILES: O=[N+]([O-])c1cc(Br)ccc1-c1nc(C(F)(F)F)c[nH]1] | 1H NMR (400 MHz, DMSO-d6): δ 13.43 (bs, 1H), 8.30 (d, J = 1.6 Hz, 1H), 8.06 (dd, J = 8.4, 1.8 Hz, 1H), 8.03 (s, 1H), 7.80 (d, J = 8.8 Hz, 1H) |
| I-56 | 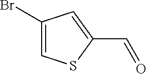 | 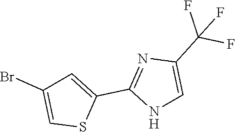 [SMILES: FC(F)(F)c1c[nH]c(-c2cc(Br)cs2)n1] | 1H NMR (400 MHz, DMSO-d6): δ 13.34 (bs, 1H), 7.95 (s, 1H), 7.77 (d, J = 1.6 Hz, 1H), 7.63 (d, J = 1.2 Hz, 1H); LC-MS: m/z 297.0 (M + H)+. |
| I-57 | 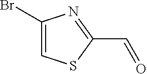 | 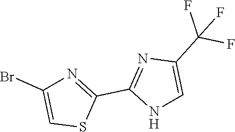 [SMILES: FC(F)(F)c1c[nH]c(-c2nc(Br)cs2)n1] | 1H NMR (400 MHz, DMSO-d6): δ 14.02 (bs, 1H), 8.01 (s, 1H), 8.00 (s, 1H); LC-MS: m/z 300.9 (M + H)+. |

### Synthesis of 2-(4-bromophenyl)-1-methyl-4-(trifluoromethyl)-1H-imidazole (I-58)

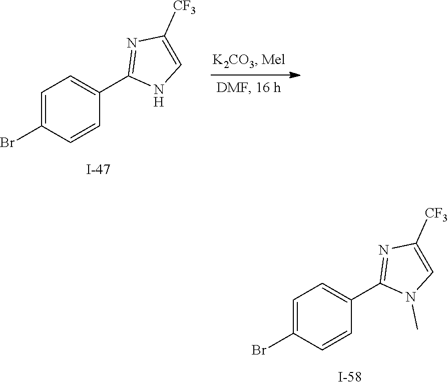 [SMILES: FC(F)(F)c1c[nH]c(-c2ccc(Br)cc2)n1 ; Cn1cc(C(F)(F)F)nc1-c1ccc(Br)cc1]

To a stirred solution of intermediate I-47 (3.0 g, 10.31 mmol, 1.0 equiv.) in DMF (100 mL) at 0° C. was added K₂CO₃ (4.27 g, 30.92 mmol, 3.0 equiv.) followed by methyl iodide (2.19 g, 15.46 mmol, 1.5 equiv.). The reaction mixture was allowed warm to room temperature and stirred for 16 hours. The reaction mixture was quenched with ice-cold water, then extracted with ethyl acetate (3×). The combined organic layers were washed with brine and dried over anhydrous sodium sulphate. The filtered organic layer was concentrated to give crude product which was purified by combi-flash column chromatography using ethyl acetate in hexane to afford the N-methylimidazole I-58 (0.8 g) as an off-white solid.

¹H NMR (400 MHz, DMSO-d₆): δ 7.93 (s, 1H), 7.68 (d, J=8.8 Hz, 2H), 7.65 (d, J=8.8 Hz, 2H), 3.76 (s, 3H)

The intermediates shown below in Table 8 were prepared according to the procedure described in I-58 or using Cs₂CO₃ as base instead of K₂CO₃ in DMF at 100° C. for around 16 hours using the appropriate alkyl halide as given here.

TABLE 8

| | | Alkyl | | |
|---|---|---|---|---|
| Int. No | SM | halide | Int. Structure | Analytical Data |
| I-59 | I-47 | 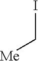 | 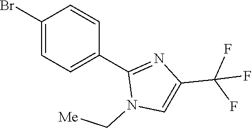 [SMILES: CCn1cc(C(F)(F)F)nc1-c1ccc(Br)cc1] | 1H NMR (400 MHz, DMSO-d6): δ 8.06 (d, J = 1.2 Hz, 1H), 7.73 (d. J = 8.4 Hz, 2H), 7.61 (d, J = 8.4 Hz, 2H), 4.10 (q, J = 7.2 Hz, 2H), 1.33 (q, J = 7.4 Hz, 3H); LC-MS: m/z 320.9 (M + H)+. |
| I-60 | I-47 | 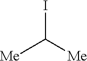 | 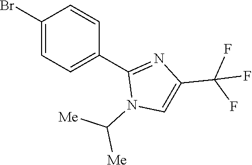 [SMILES: CC(C)n1cc(C(F)(F)F)nc1-c1ccc(Br)cc1] | 1H NMR (400 MHz, DMSO-d6): δ 8.21 (s. 1H), 7.73 (d, J = 8.4 Hz, 2H), 7.53 (d, J = 8.8 Hz, 2H), 4.67 (septet, J = 6.6 Hz, 1H), 1.41 (d, J = 6.8 Hz, 6H); LC-MS: m/z 334.9 (M + H)+. |
| I-61 | I-47 | 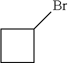 | 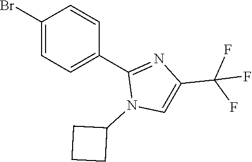 [SMILES: FC(F)(F)c1cn(C2CCC2)c(-c2ccc(Br)cc2)n1] | 1H NMR (400 MHz, DMSO-d6): δ 8.26 (d, J = 1.2 Hz, 1H), 7.69 (d, J = 8.4 Hz, 2H), 7.50 (d, J = 8.4 Hz, 2H), 4.78-4.69 (m, 1H), 2.41-2.30 (m, 4H), 1.75-1.62 (m, 2H); LC-MS: m/z 344.9 (M + H)+. |
| I-62 | I-47 | 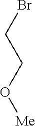 | 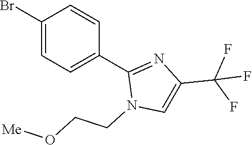 [SMILES: COCCn1cc(C(F)(F)F)nc1-c1ccc(Br)cc1] | 1H NMR (400 MHz, DMSO-d6): δ 7.97 (d, J = 0.8 Hz, 1H), 7.69 (d, J = 8.8 Hz, 2H), 7.59 (d, J = 8.8 Hz, 2H), 4.19 (t, J = 5.6 Hz, 2H), 3.60 (t, J = 5.6 Hz, 2H), 3.15 (s, 3H); LC-MS: m/z 348.9 (M + H)+. |
| I-63 | I-47 | 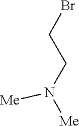 | 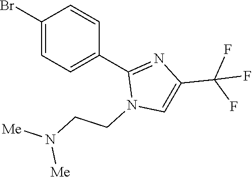 [SMILES: CN(C)CCn1cc(C(F)(F)F)nc1-c1ccc(Br)cc1] | 1H NMR (400 MHz, DMSO-d6): δ 8.00 (s, 1H), 7.69 (d, J = 8.4 Hz, 2H), 7.58 (d, J = 8.8 Hz, 2H), 4.10 (t, J = 6.2 Hz, 2H), 2.49 (t, J = 6.0 Hz, 2H), 2.02 (s, 6H); LC-MS: m/z 362.0 (M + H)+. |
| I-64 | I-47 | 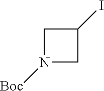 [SMILES: CC(C)(C)OC(=O)N1CC(I)C1] | 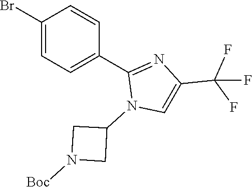 [SMILES: CC(C)(C)OC(=O)N1CC(n2cc(C(F)(F)F)nc2-c2ccc(Br)cc2)C1] | 1H NMR (400 MHz, DMSO-d6): δ 8.48 (d, J = 1.2 Hz, 1H), 7.73 (d, J = 8.4 Hz, 2H), 7.52 (d, J = 8.4 Hz, 2H), 5.21-5.15 (m, 1H), 4.35-4.20 (m, 2H), 4.15-4.08 (m, 2H), 1.40 (s, 9H); LC-MS: m/z 447.8 (M + H)+. |
| I-65 | I-48 | MeI | 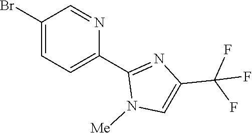 [SMILES: Cn1cc(C(F)(F)F)nc1-c1ccc(Br)cn1] | 1H NMR (400 MHz, DMSO-d6): δ 8.79 (d, J = 2.0 Hz, 1H), 8.17 (dd, J = 8.8, 2.0 Hz, 1H), 8.02 (d, J = 8.4 Hz, 1H), 8.02 (s, 1H), 4.05 (s, 3H); LC-MS: m/z 305,8 (M + H)+ |
| I-66 | I-49 | MeI | 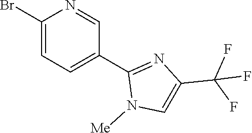 [SMILES: Cn1cc(C(F)(F)F)nc1-c1ccc(Br)nc1] | 1H NMR (400 MHz, DMSO-d6): δ 8.74 (d, J = 2.8 Hz, 1H), 8.11 (dd, J = 8.2, 2.6 Hz, 1H), 8.03 (s, 1H), 7.81 (d, J = 8.4 Hz, 1H), 3.82 (s, 3H); LC-MS: m/z 307.9 (M + H)+ |
| I-67 | I-49 | 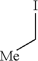 | 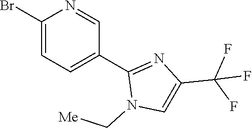 [SMILES: CCn1cc(C(F)(F)F)nc1-c1ccc(Br)nc1] | 1H NMR (400 MHz, DMSO-d6): δ 8.67 (dd, J = 2.4, 0.4 Hz, 1H), 8.14 (d, J = 1.2 Hz, 1H), 8.05 (dd, J = 8.4. 2.4 Hz, 1H), 7.83 (dd, J = 8.4, 0.8 Hz, 1H), 4.12 (q, J = 7.4 Hz, 2H), 1.35 (t, J = 7.4 Hz, 3H); LC-MS: m/z 321.9 (M + H)⁺. |
| I-68 | I-49 | 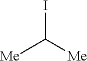 | 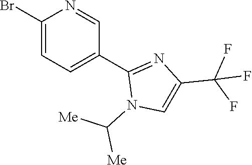 [SMILES: CC(C)n1cc(C(F)(F)F)nc1-c1ccc(Br)nc1] | 1H NMR (400 MHz, DMSO-d6): δ 8.61 (dd, J = 2.4, 0.4 Hz, 1H), 8.29 (d, J = 1.2 Hz, 1H), 7.98 (dd, J = 8.4, 2.8 Hz, 1H), 7.83 (dd, J = 8.4, 0.8 Hz, 1H), 4.45 (septet, J = 6.6 Hz, 1H), 1.43 (t, J = 6.4 Hz, 6H); LC-MS: m/z 334.0 (M + H)+. |
| I-69 | I-49 | 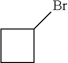 | 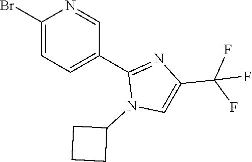 [SMILES: FC(F)(F)c1cn(C2CCC2)c(-c2ccc(Br)nc2)n1] | LC-MS: m/z 347.9 (M + H)+. |
| I-70 | I-50 | MeI | 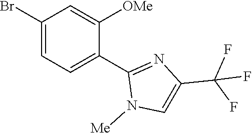 [SMILES: COc1cc(Br)ccc1-c1nc(C(F)(F)F)cn1C] | 1H NMR (400 MHz, Chloroform-d): δ 7.51 (s, 1H), 7.42-7.10 (m, 3H), 3.87 (s, 3H), 3.56 (s, 3H); LC-MS: m/z 334.9 (M + H)+ |
| I-71 | I-51 | MeI | 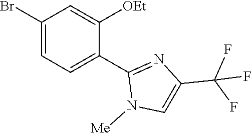 [SMILES: CCOc1cc(Br)ccc1-c1nc(C(F)(F)F)cn1C] | 1H NMR (400 MHz, Chloroform-d): δ 7.37 (d, J = 8.0 Hz. 1H), 7.33 (s, 1H), 7.32-7.12 (m, 2H), 4.11 (q, J = 6.8 Hz, 2H), 3.58 (s, 3H), 1.37 (t, J = 6.8 Hz 3H); LC-MS: m/z 349.0 (M + H)+. |
| I-72 | I-52 | MeI | 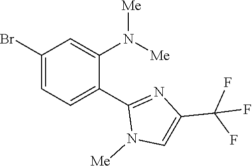 [SMILES: CN(C)c1cc(Br)ccc1-c1nc(C(F)(F)F)cn1C] | 1H NMR (400 MHz, Chloroform-d): δ 7.93 (d, J = 1.6 Hz, 1H), 7.24-7.12 (m, 3H), 3.46 (s, 3H), 2.47 (s, 6H); LC-MS: m/z 348.0 (M + H)+. |
| I-73 | I-53 | MeI | 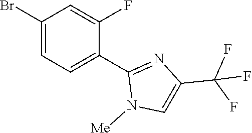 [SMILES: Cn1cc(C(F)(F)F)nc1-c1ccc(Br)cc1F] | 1H NMR (400 MHz, DMSO-d6): δ 8.03 (s, 1H), 7.81 (d, J = 8.4 Hz, 1H), 7.65-7.57 (m, 2H), 3.62 (s, 3H); LC-MS: m/z 323.0 (M + H)+. |
| I-74 | I-54 | Mel | 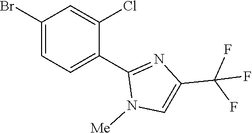 [SMILES: Cn1cc(C(F)(F)F)nc1-c1ccc(Br)cc1Cl] | 1H NMR (400 MHz, DMSO-d6): δ 8.02 (s, 1H), 8.01 (s, 1H), 7.78-7.50 (m, 2H), 3.53 (s, 3H); LC-MS: m/z 340.9 (M + H)+. |
| I-75 | I-55 | MeI | 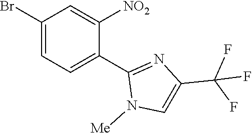 [SMILES: Cn1cc(C(F)(F)F)nc1-c1ccc(Br)cc1[N+](=O)[O-]] | 1H NMR (400 MHz, DMSO-d6): δ 8.40 (d, J = 2.0 Hz, 1H), 8.13 (dd, J = 7.8, 2.2 Hz, 1H), 8.04 (d, J = 1.2 Hz, 1H), 7.78 (d, J = 8.0 Hz, 1H), 3.60 (s, 3H). |
| I-76 | I-56 | MeI | 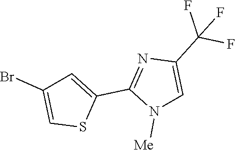 [SMILES: Cn1cc(C(F)(F)F)nc1-c1cc(Br)cs1] | 1H NMR (400 MHz, DMSO-d6): δ 7.98 (d, J = 1.2 Hz, 1H), 7.86 (d, J = 1.2 Hz, 1H), 7.61 (d, J = 1.2 Hz, 1H), 3.89 (s, 3H); LC-MS: m/z 310.8 (M + H)+. |
| I-77 | I-57 | MeI | 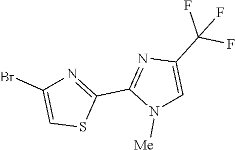 [SMILES: Cn1cc(C(F)(F)F)nc1-c1nc(Br)cs1] | 1H NMR (400 MHz, DMSO-d6): δ 8.14 (s, 1H), 8.02 (s, 1H), 4.07 (s, 3H); LC-MS: m/z 313.9 (M + H)+. |

### Synthesis of 2-(4-bromophenyl)-1-cyclopropyl-4-(trifluoromethyl)-1H-imidazole (I-78)

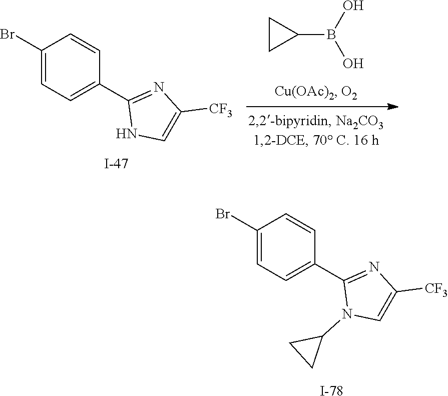

The intermediate I-78 was synthesized from starting materials I-47 and cyclopropylboronic acid according to the procedure described for synthesis of intermediate I-21.

¹H NMR (400 MHz, DMSO-d₆): δ7.98 (s, 1H), 7.85 (d, J=8.4 Hz, 2H), 7.70 (d, J=9.2 Hz, 2H), 3.79-3.63 (m, 1H), 1.02-0.98 (m, 2H), 0.91-0.86 (m, 2H)

### Synthesis of 1-(4-bromophenyl)-3,5-dimethyl-1H-pyrazole (1-79)

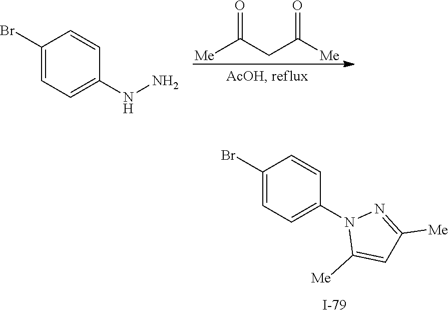

To a stirred solution of (4-bromophenyl)hydrazine (1.0 g, 5.35 mmol, 1.0 equiv.) in acetic acid (20 mL) at 0° C. was added acetylacetone (0.7 g, 6.94 mmol, 1.3 equiv.) dropwise. After the addition, the mixture was heated to reflux for 4 hours. The reaction mixture was cooled to room temperature and concentrated to give crude product which was purified by combi-flash column chromatography using ethyl acetate in hexane as an eluent to afford the pyrazole I-79 (1.4 g) as a brown solid.

¹H NMR (400 MHz, DMSO-d₆): δ 7.67 (d, J=8.4 Hz, 2H), 7.47 (d, J=8.8 Hz, 2H), 6.09 (s, 1H), 2.30 (s, 3H), 2.18 (s, 3H); LCMS 251.0 m/z (M+H)⁺

The intermediates in Table 9 were prepared from (4-bromophenyl)hydrazine according to the procedure described in I-79, using the appropriate starting material as given here.

TABLE 9

| Int. No. | SM | Int. Structure | Analytical Data |
|---|---|---|---|
| I-80 | 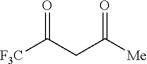 | 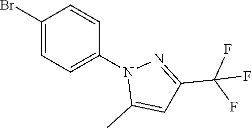 [SMILES: Cc1cc(C(F)(F)F)nn1-c1ccc(Br)cc1] | LC-MS: 306.8 m/z (M + H)+ |
| I-81 | 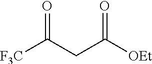 | 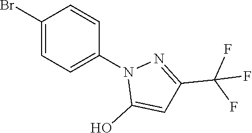 [SMILES: Oc1cc(C(F)(F)F)nn1-c1ccc(Br)cc1] | 1H NMR (400 MHz, DMSO-d6): δ 12.6 (bs, 1H), 7.67 (s, 4H), 5.91 (s, 1H); LC-MS: 308.7 m/z (M + H)+ |

### Synthesis of 1-(4-bromophenyl)-5-ethoxy-3-(trifluoromethyl)-1H-pyrazole (I-82)

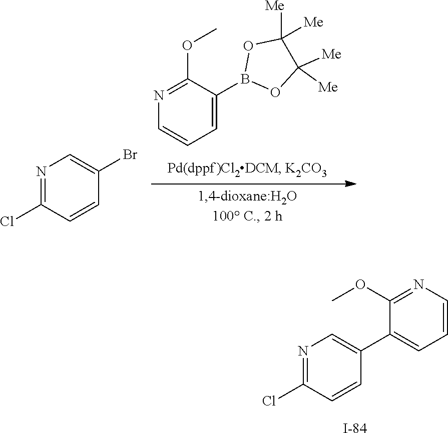

To a stirred solution of intermediate 1-81 (0.2 g, 0.651 mmol, 1.0 equiv.) in DMF (4 mL) was added sodium hydride (0.022 g, 0.97 mmol, 1.5 equiv.) at 0° C. and the mixture was stirred for 30 minutes. After that, at the same temperature ethyl iodide (0.112 g, 0.71 mmol, 1.1 equiv.) was added. The reaction mixture was allowed to warm to room temperature and stirred for 16 h. The reaction mixture was quenched with ice-cold water, then extracted with ethyl acetate (3×). The combined organic layers were washed with brine and dried over anhydrous sodium sulphate. The filtered organic layer was concentrated to give crude product which was purified by combi-flash column chromatography using ethyl acetate in hexane to give 1-82 (0.15 g) as an off-white solid.

¹H NMR (400 MHz, DMSO-d₆): δ 7.73 (d, J=8.8 Hz, 2H), 7.65 (d, J=8.8 Hz, 2H), 6.49 (s, 1H), 4.30 (q, J=6.8 Hz, 2H), 1.37 (t, J=7.2 Hz, 3H).

### Synthesis of 1-(4-bromophenyl)-5-methoxy-3-(trifluoromethyl)-1H-pyrazole (1-83)

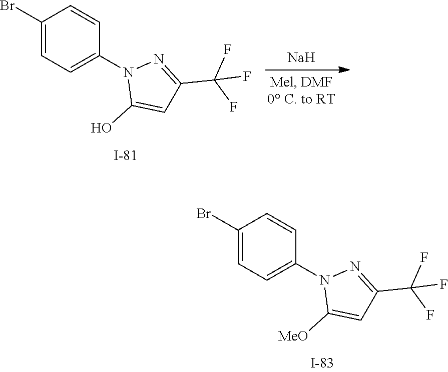

The intermediate in I-83 was prepared according to the procedure described in I-82 from starting materials I-81 and methyl iodide.

¹H NMR (400 MHz, DMSO-d₆): δ 7.75 (d, J=8.8 Hz, 2H), 7.66 (d, J=8.8 Hz, 2H), 6.51 (s, 1H), 4.03 (s, 3H).

### Synthesis of 6′-chloro-2-methoxy-3,3′-bipyridine (1-84)

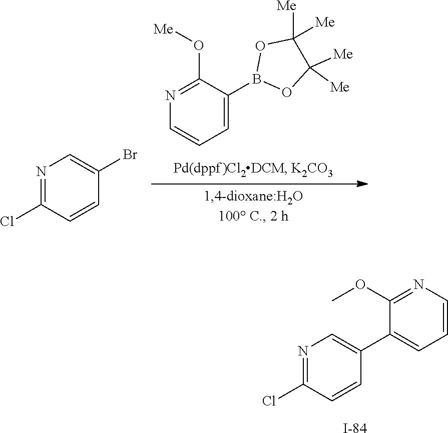

The compound 1-84 was synthesized from commercially available starting materials 5-bromo-2-chloropyridine and 2-methoxy-3-(4,4,5,5-tetramethyl-1,3,2-dioxaborolan-2-yl)pyridine according to the procedure described for synthesis of intermediate 1-23. However, the reaction time was reduced to 2 hours.

¹H NMR (400 MHz, DMSO-d₆): δ 8.79 (dd, J=2.8, 0.8 Hz, 1H), 8.25 (dd, J=8.8, 1.8 Hz, 1H), 8.08 (dd, J=8.2, 2.6 Hz, 1H), 7.88 (dd, J=7.4, 1.8 Hz, 1H), 7.61 (dd, J==8.4, 0.8 Hz, 1H), 7.15 (dd, J=7.4, 3.0 Hz, 1H), 3.91 (s, 3H); LC-MS: m/z 221.0 (M+H)⁺

### Synthesis of 4-(6-chloropyridin-3-yl)-3,5-dimethylisoxazole (1-85)

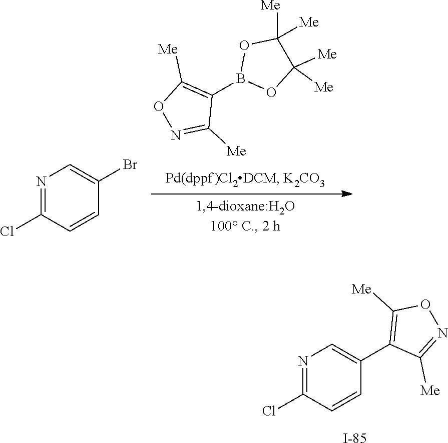

The compound 1-85 was synthesized from commercially available starting materials 5-bromo-2-chloropyridine and 3,5-dimethyl-4-(4,4,5,5-tetramethyl-1,3,2-dioxaborolan-2-yl)isoxazole according to the procedure described for synthesis of intermediate I-23. However, the reaction time was reduced to 2 hours.

¹H NMR (400 MHz, DMSO-d₆): δ 8.48 (dd, J=2.6, 0.6 Hz, 1H) 7.94 (dd, J=8.2, 2.6 Hz, 1H), 7.64 (dd, J=8.2, 0.6 Hz, 1H), 2.43 (s, 3H), 2.26 (s, 3H); LC-MS: m/z 209.0 (M+H)⁺

### Synthesis of 5-(1-methyl-4-(trifluoromethyl)-1H-imidazol-2-yl)pyrazin-2-amine (I-86)

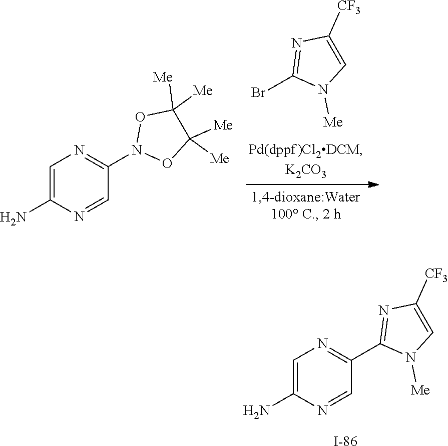

The compound 1-86 was synthesized from commercially available starting materials 5-(4,4,5,5-tetramethyl-1,3,2-dioxaborolan-2-yl)pyrazin-2-amine and 2-bromo-1-methyl-4-(trifluoromethyl)-1H-imidazole according to the procedure described for synthesis of intermediate 1-23. However, the reaction time was reduced to 2 hours.

¹H NMR (400 MHz, DMSO-d₆): δ 8.50 (d, J=1.2 Hz, 1H), 7.90 (d, J=1.2 Hz, 1H), 7.89 (d, J=1.2 Hz, 1H), 6.85 (s, 2H), 3.93 (s, 3H); LC-MS: m/z 244.1 (M+H)⁺

### Synthesis of 2-chloro-5-(1-methyl-4-(trifluoromethyl)-1H-imidazol-2-yl)pyrazine (I-87)

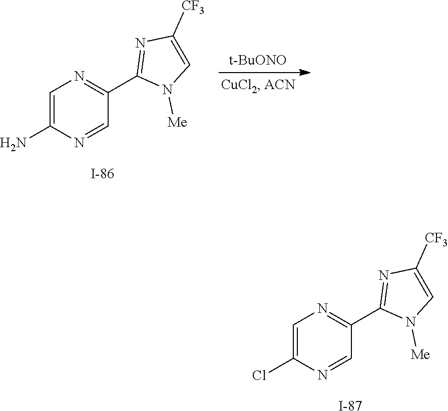

To a stirred solution of intermediate I-86 (0.08 g, 0.329 mmol, 1.0 equiv.) in acetonitrile (4 mL) was added tert-butylnitrite (0.068 g, 0.66 mmol, 2.01 equiv.) at 0° C. and the reaction mixture was stirred for 10 minutes at the same temperature. After that, copper(II) chloride (0.11 g, 0.82 mmol, 2.50 equiv.) was added and the reaction mixture was heated to 55° C. for 16 hours. The reaction mixture was cooled to room temperature, quenched with ice-cold water, and extracted with ethyl acetate (3×). The combined organic layers were washed with brine and dried over anhydrous sodium sulphate. The filtered organic layer was concentrated to give crude product which was purified by combi-flash column chromatography using ethyl acetate in hexane as an eluent to afford the product 1-87 (0.04 g) as a brown solid.

¹H NMR (400 MHz, DMSO-d₆): δ 9.09 (d, J=1.6 Hz, 1H), 8.90 (d, J=1.6 Hz, 1H), 8.14 (d 1=0.8 Hz, 1H), 4.05 (s, 3H); LC-MS: m/z 263.0 (M+H)⁺

### Synthesis of 5-bromo-2-(5-methyl-3-(trifluoromethyl)-1H-pyrazol-1-yl)pyrimidine (I-88)

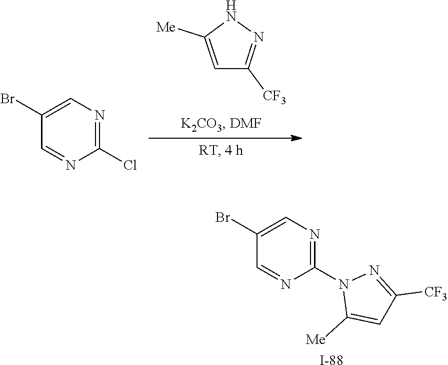

A mixture of 5-bromo-2-chloropyrimidine (0.3 g, 1.55 mmol, 1.0 equiv.), 5-methyl-3-(trifluoromethyl)-1H-pyrazole (0.35 g, 2.33 mmol, 1.5 equiv.) and K₂CO₃ (0.64 g, 4.65 mmol, 3.0 equiv.) in DMF (5 mL) and stirred at room temperature for 4 hours. The reaction mixture was quenched with ice-cold water, then extracted with ethyl acetate (3×). The combined organic layers were washed with brine and dried over anhydrous sodium sulphate. The filtered organic layer was concentrated to give crude product which was purified by combi-flash column chromatography using ethyl acetate in hexane to afford 1-88 (0.4 g) as a white solid.

¹H NMR (400 MHz, DMSO-d₆): δ 9.16 (s, 2H), 6.86 (s, 1H), 2.63 (s, 3H); LC-MS: m/z 309.0 (M+H)⁺

### Synthesis of intermediate 1-91

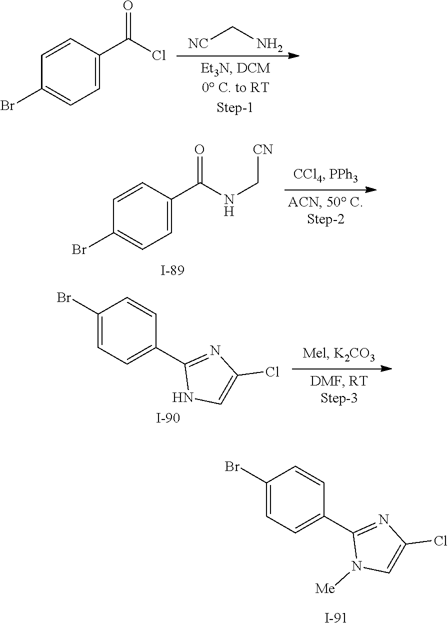

### Step-1: Synthesis of 4-bromo-N-(cyanomethyl)benzamide (I-89)

To a mixture of 2-aminoacetonitrile (1.0 g, 17.84 mmol, 1.0 equiv.) and triethylamine (5.41 g, 53.5 mmol, 3.0 equiv.) in dichloromethane (10 mL) was added 4-bromobenzoyl chloride (0.08 g, 4.46 mmol, 0.25 equiv., commercial source) at 0° C. The reaction mixture was allowed to warm to room temperature and stirred for 16 hours. The reaction mixture was quenched with ice-cold water, then extracted with ethyl acetate (3×). The combined organic layers were washed with brine and dried over anhydrous sodium sulphate. The filtered organic layer was concentrated to give crude product amide I-89 (1.0 g) as a brown solid.

¹H NMR (400 MHz, Chloroform-d): δ 7.71-7.62 (m, 4H), 6.50 (bs, 1H), 4.41 (d, J=5.6 Hz, 2H)

LC-MS: m/z 238.9 (M+H)⁺

### Step-2: Synthesis of 2-(4-bromophenyl)-4-chloro-1H-imidazole (I-90)

To a stirred solution of amide I-89 (1.0 g, 4.18 mmol, 1.0 equiv.) in acetonitrile (15 mL) was added triphenyl phosphine (2.74 g, 10.45 mmol, 2.5 equiv.) followed by carbon tetrachloride (1.61 g, 10.45 mmol, 2.5 equiv.) at room temperature. The reaction mixture was heated to 50° C. and stirred for 16 hours. The reaction mixture was quenched with ice-cold water, then extracted with ethyl acetate (3×). The combined organic layers were washed with brine and dried over anhydrous sodium sulphate. The filtered organic layer was concentrated to give crude product, which was purified by combi-flash chromatography using ethyl acetate in hexane as mobile phase to give pure chloroimidazole I-90 (0.1 g) as an off-white solid.

¹H NMR (400 MHz, DMSO-d₆): δ 12.95 (bs, 1H), 7.83 (d, J=8.8 Hz, 2H), 7.67 (d, J=8.8 Hz, 2H), 7.40 (s, 1H); LC-MS: m/z 258.9 (M+H)⁺

### Step-3: Synthesis of 2-(4-bromophenyl)-4-chloro-1-methyl-1H-imidazole (I-91)

The compound I-91 was synthesized from I-90 according to the procedure described for synthesis of I-58.

¹H NMR (400 MHz, DMSO-d₆): δ 7.69 (d, J=84 Hz, 2H), 7.63 (d, J=8.0 Hz, 2H), 7.40 (s, 1H), 3.72 (s, 3H); LC-MS: m/z 272.9 (M+H)⁺

### Synthesis of tert-butyl 3-(4-bromophenyl)-1-methyl-1,4,6,7-tetrahydro-5H-pyrazolo[4,3-c]pyridine-5-carboxylate (I-92)

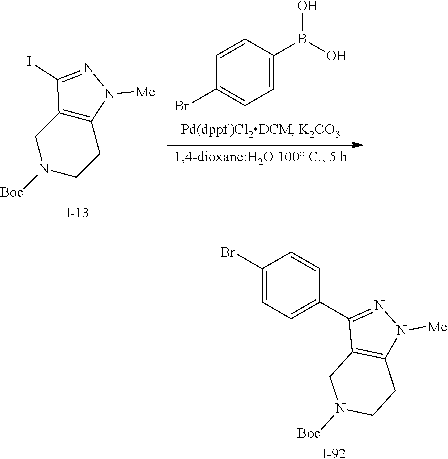

The compound I-92 was synthesized from starting materials I-13 and (4-bromophenyl)boronic acid according to the procedure described for synthesis of I-23.

LC-MS: m/z 392.2 (M+H)⁺

### 3-(4-bromophenyl)-1,5-dimethyl-4,5,6,7-tetrahydro-1H-pyrazolo[4,3-c]pyridine (I-93)

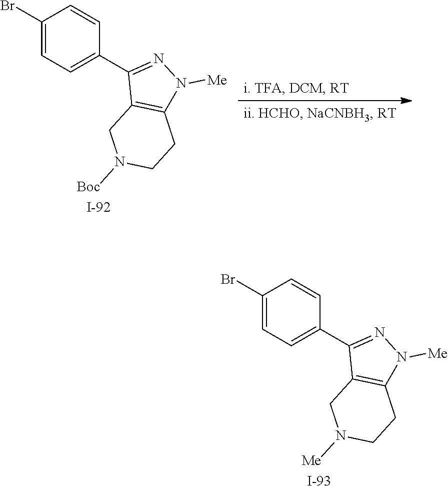

The intermediate I-92 (0.30 g) was subjected to procedure described for I-36 to give a crude free amine (0.15 g).

To a mixture of the crude product (0.15 g, 0.513 mmol, 1.0 equiv.) and formaldehyde (0.039 g, 1.28 mmol, 2.50 equiv.) in methanol (6 mL) was added acetic acid (0.003 g, 0.050 mmol, 0.1 equiv.) at 0° C. The reaction mixture was heated to 80° C. and stirred for 1 hour at same temperature. After that, the reaction mixture was cooled to 0° C. and sodium cyanoborohydride (0.081 g, 1.28 mmol, 2.50 equiv.) was added. The reaction mixture was heated to 60° C. and stirred for 2 hours. The reaction mixture was cooled to room temperature and quenched with ice-cold water, then extracted with ethyl acetate (3×). The combined organic layers were washed with brine and dried over anhydrous sodium sulphate. The filtered organic layer was concentrated to give crude product which was purified by combi-flash column chromatography using ethyl acetate in hexane as an eluent to afford tertiary amine I-93 (0.09 g).

LC-MS: m/z 306.2 (M+H)⁺

### Synthesis of 2-(4-bromophenyl)-1-(1-methylazetidin-3-yl)-4-(trifluoromethyl)-1H-imidazole (I-94)

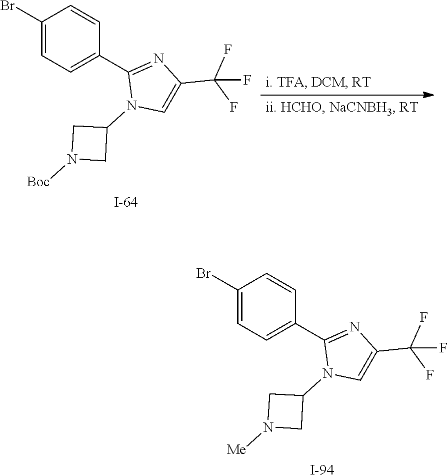

The intermediate I-94 was prepared according to the procedure described in I-93 from starting material 1-64.

LC-MS: m/z 359.9 (M+H)⁺

### Synthesis of intermediate I-98

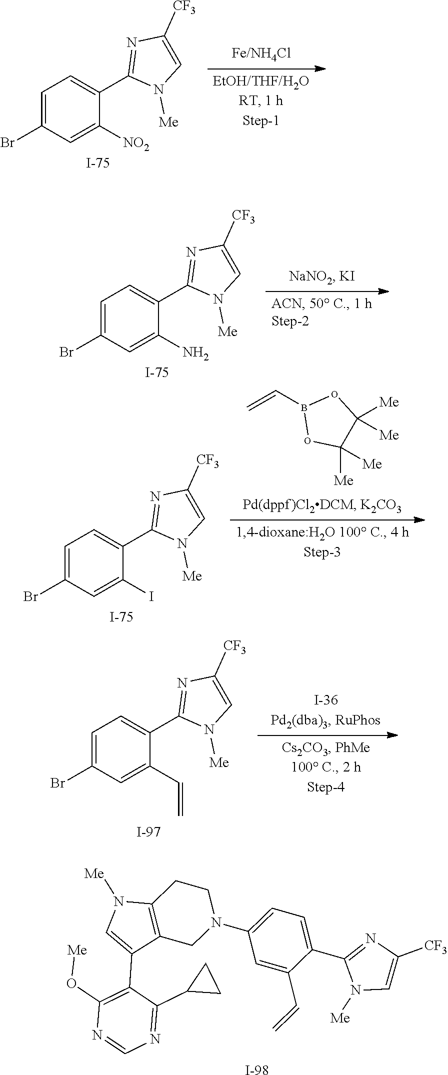

### Step-1: Synthesis of 5-bromo-2-(1-methyl-4-(trifluoromethyl)-1H-imidazol-2-yl)aniline (I-95)

To a stirred solution of nitrobenzene I-75 (2.0 g, 5.71 mmol, 1.0 equiv.) in a mixture of EtOH (30 mL). THF (30 mL) and water (10 mL), were added iron powder (1.60 g, 28.65 mmol, 5.0 equiv.) and ammonium chloride (1.53 g, 28.56 mmol, 5.0 equiv.). The reaction mixture was then heated to 80° C. for 1 h. The reaction mixture was cooled to room temperature and filtered through a pad of Celite®. The filtrate was dried over anhydrous sodium sulphate, and then concentrated to give crude product, which was purified by combi-flash chromatography using ethyl acetate and hexane as a mobile phase to afford aniline I-95 (1.4 g) as brown solid.

¹H NMR (400 MHz, DMSO-d₆): δ 7.94 (s, 1H), 7.19 (d, J=8.0 Hz, 1H), 7.02 (d, J=2.4 Hz, 1H), 6.77 (dd, J=8.0, 2.4 Hz, 1H), 5.95 (s, 2H), 3.64 (s, 3H); LC-MS m/z 319.9 (M+H)⁺

### Step-2: Synthesis of 2-(4-bromo-2-iodophenyl)-1-methyl-4-(trifluoromethyl)-1H-imidazole (I-96)

To aniline 1-95 (0.2 g, 0.625 mmol, 1.0 equiv.) were added IM hydrochloride (4 mL) and water (3.5 mL) at 0° C. and the mixture was stirred for 1 hour. At the same temperature, a solution of sodium nitrite (0.172 g, 2.49 mmol, 3.99 equiv.) in water (2 mL) was added dropwise to the reaction mixture and the mixture was stirred for 1 hour. After that, potassium iodide (0.623 g, 3.75 mmol, 6.0 equiv.) in water (4 mL) was added dropwise and the reaction mixture was stirred for another 6 hours. The reaction mixture was quenched with aq. sodium thiosulphate and extracted with ethyl acetate (3×). The combined organic layers were washed with brine and dried over anhydrous sodium sulphate. The filtered organic layer was concentrated to give crude product. The crude material was washed with diethyl ether and hexane to give iodobenzene 1-96 (0.12 g) as an off-white solid.

¹H NMR (400 MHz, DMSO-d₆): δ 8.21 (d, J=2.0 Hz, 1H), 7.94 (s, 1H), 7.72 (dd, J=8.4, 2.0 Hz 1H), 7.38 (d, J=8.0 Hz, IH), 3.43 (s, 3H)

### Step-3: 2-(4-bromo-2-vinylphenyl)-1-methyl-4-(trifluoromethyl)-1H-imidazole (I-97)

The intermediate I-97 was synthesized from the starting materials I-96 and 4,4,5,5-tetramethyl-2-vinyl-1,3,2-dioxaborolane according to the procedure described for synthesis of intermediate I-23. However, the reaction time was reduced to 4 hours.

¹H NMR (400 MHz, DMSO-d₆): δ 798 (d, J=2.0 Hz, IH), 7.95 (s, 1H), 7.59 (dd, J=8.0, 2.0 Hz, 1H), 7.36 (d, J=8.4 Hz, TH), 6.38 (dd, J=13.6, 11.2 Hz, 1H), 5.85 (d, J=13.6 Hz, 1H), 5.33 (d, J=11.2 Hz, 1H), 3.42 (s, 3H); LC-MS: m/z 330.9 (M+H)⁺

### Step-4:3-(4-cyclopropyl-6-methoxypyrimidin-5-yl)-1-methyl-5-(4-(1-methyl-4-(trifluoromethyl)-1H-imidazol-2-yl)-3-vinylphenyl)-4,5,6,7-tetrahydro-1H-pyrazolo[4,3-c]pyridine (I-98)

A mixture of intermediate I-97 (0.050 g, 0.151 mmol, 1.0 equiv.), piperidine I-36 (0.047 g, 0.166 mmol, 1.1 equiv.), RuPhos (0.014 g, 0.01 mmol, 0.1 equiv.) and cesium carbonate (0.148 g, 0.45 mmol, 3.0 eq) in toluene (3 mL) was degassed with argon for 10 minutes. After that, Pd₂(dba)₃ (0.014 g, 0.03 mmol, 0.2 equiv.) was added and the reaction mixture was heated to 100° C. for 2 hours. The reaction mixture was cooled to room temperature and quenched with ice-cold water, then extracted with ethyl acetate (3×). The combined organic layers were washed with brine and dried over anhydrous sodium sulphate. The filtered organic layer was concentrated to give crude product which was purified by combi-flash column chromatography using ethyl acetate in hexane as an eluent to afford the coupled product I-98 (0.011 g) as an off-white solid.

LC-MS: m/z 536.1 (M+H)⁺

### Synthesis of Intermediate I-101

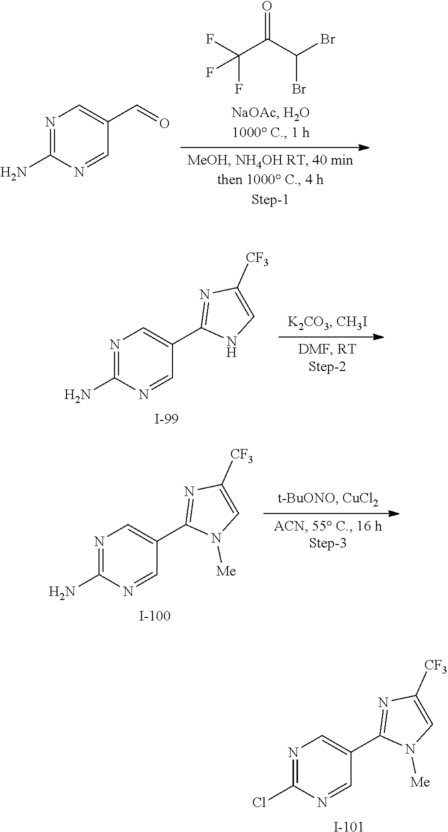

### Step-1: Synthesis of 5-(4-(trifluoromethyl)-1H-imidazol-2-yl)pyrimidin-2-amine (I-99)

The intermediate I-99 was synthesized from the starting materials 2-aminopyrimidine-5-carbaldehyde and 3,3-dibromo-1,1,1-trifluoropropan-2-one according to the procedure described for synthesis of I-47.

¹H NMR (400 MHz, DMSO-d₆): δ 13.00 (bs, 1H), 8.69 (s, 2H) 7.89 (s, 1H), 7.06 (s, 2H); LC-MS: m/z 230.1 (M+H)⁺

### Step-2: Synthesis of 5-(1-methyl-4-(trifluoromethyl)-1H-imidazol-2-yl)pyrimidin-2-amine (I-100)

The intermediate I-100 was synthesized from the starting material I-99 according to the procedure described for synthesis of I-58.

¹H NMR (400 MHz, DMSO-d₆): δ 8.52 (s, 2H), 7.88 (s, 1H), 7.07 (s, 2H), 3.72 (s, 3H); LC-MS: m/z 244.1 (M+H)⁺

### Step-3: Synthesis of 2-chloro-5-(1-methyl-4-(trifluoromethyl)-1H-imidazol-2-yl)pyrimidine (I-101)

The intermediate I-101 was synthesized from the starting material I-100 according to the procedure described for synthesis of I-87.

¹H NMR (400 MHz, DMSO-do): δ 9.12 (s, 2H), 8.10 (s, 1H), 3.87 (s, 3H); LC-MS: m/z 263.0 (M+H)⁺

### EXAMPLES

### Example 1: 3-(4-cyclopropyl-6-methoxypyrimidin-5-yl)-1-methyl-5-(4-(1-methyl-4-(trifluoromethyl)-1H-imidazol-2-yl)phenyl)-4,5,6,7-tetrahydro-1H-pyrazolo[4,3-c]pyridine (Compound-1)

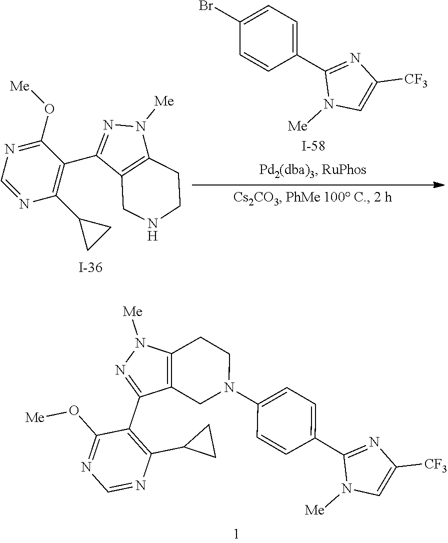

A mixture of piperidine I-36 (0.05 g, 0.175 mmol, 1.0 equiv.), bromobenzene I-58 (0.053 g, 0.175 mmol, 1.0 equiv.), RuPhos (0.008 g, 0.01 mmol, 0.1 equiv.) and cesium carbonate (0.114 g, 0.35 mmol, 2.0 eq) in toluene (5 mL) was degassed with argon for 10 minutes. After that, Pd₂(dba)₃ (0.016 g, 0.01 mmol, 0.1 equiv.) was added and the reaction mixture was heated to 100° C. for 2 hours. The reaction mixture was cooled to room temperature and quenched with ice-cold water, then extracted with ethyl acetate (3×). The combined organic layers were washed with brine and dried over anhydrous sodium sulphate. The filtered organic layer was concentrated to give crude product which was purified by prep-TLC using ethyl acetate in hexane as a mobile phase to afford the coupled product 1 (0.007 g) as an off-white solid.

¹H NMR (400 MHz, DMSO-d₆): δ 8.62 (s, 1H), 7.84 (s, 1H), 7.52 (d, J=8.8 Hz, 2H), 7.07 (d. J=9.2 Hz, 2H), 4.14 (s, 2H), 3.91 (s, 3H), 3.77 (t, J=5.6 Hz, 2H), 3.75 (s, 3H), 3.73 (s, 3H), 2.86 (t, J=5.6 Hz, 2H), 2.18-2.12 (m, 1H), 1.06-1.02 (m, 2H), 0.94-0.89 (m, 2H); LC-MS: m/z 510.2 (M+H)⁺

The compounds in Table 10 were prepared according to the procedure described for compound 1, using the appropriate starting material as given here.

TABLE 10

| Compound | | | | |
|---|---|---|---|---|
| No. | SM1 | SM2 | Compound Structure | Analytical Data |
| 2. | I-44 | I-58 | 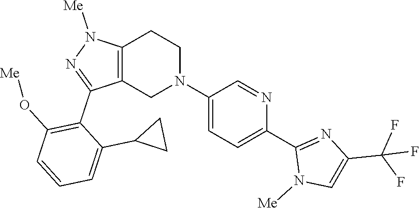 [SMILES: COc1cccc(C2CC2)c1-c1nn(C)c2c1CN(c1ccc(-c3nc(C(F)(F)F)cn3C)nc1)CC2] | ¹H NMR (400 MHz, DMSO-d₆): δ 7.83 (s, 1H), 7.52 (d, J = 8.8 Hz, 2H), 7.26 (t, J = 8.0 Hz, 1H), 7.04 (d, J = 9.2 Hz, 2H), 6.87 (d, J = 7.6 Hz, 1H), 6.47 (d, J = 7.2 Hz, 1H), 4.05 (s, 2H), 3.76 (t, J = 5.6 Hz, 2H), 3.74 (s, 3H), 3.72 (s, 3H), 3.71 (s, 3H), 2.84 (t, J = 5.6 Hz, 2H), 1.85-1.79 (m, 1H), 0.78-0.73 (m, 2H), 0.62-0.59 (m, 2H). LC-MS: m/z 508.4 (M + H)⁺ |
| 3. | I-45 | I-58 | 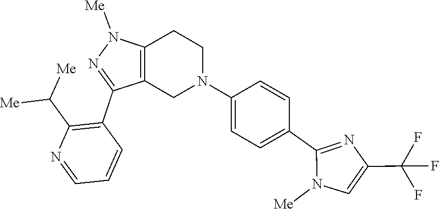 [SMILES: CC(C)c1ncccc1-c1nn(C)c2c1CN(c1ccc(-c3nc(C(F)(F)F)cn3C)cc1)CC2] | ¹H NMR (400 MHz, DMSO-d₆): δ 8.57 (dd, J = 4.8, 2.0 Hz, 1H), 8.39 (d, J = 3.2 Hz, 1H), 7.64 (dd, J = 7.8, 2.0 Hz, 1H), 7.52 (d, J = 8.8 Hz, 2H), 7.29 (dd, J = 7.8, 4.8 Hz, 1H), 7.06 (d, J = 8.8 Hz, 2H), 4.21 (s, 2H), 3.77 (t, J = 5.6 Hz, 2H), 3.77 (s, 3H), 3.73 (s, 3H), 3.49-3.40 (m, 1H), 2.88 (t, J = 5.6 Hz, 2H), 1.17 (d, J = 6.8 Hz, 6H). LC-MS: m/z 481.1 (M + H)⁺ |
| 4. | I-45 | I-65 | 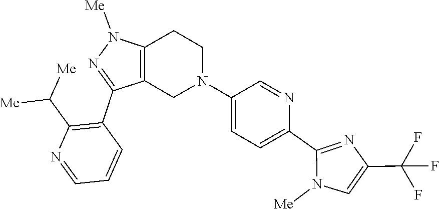 [SMILES: CC(C)c1ncccc1-c1nn(C)c2c1CN(c1ccc(-c3nc(C(F)(F)F)cn3C)nc1)CC2] | ¹H NMR (400 MHz, DMSO-d₆): δ 8.57 (dd, J = 4.8, 1.6 Hz, 1H), 8.39 (d, J = 3.2 Hz, 1H), 7.87 (d, J = 0.8 Hz, 1H), 7.85 (d, J = 8.8 Hz, 1H), 7.64 (dd, J = 7.8, 2.0 Hz, 1H), 7.49 (dd, J = 9.0, 3.2 Hz, 1H), 7.29 (dd, J = 7.6, 0.8 Hz, 1H), 4.27 (s, 2H), 4.02 (s, 3H), 3.83 (t, J = 5.6 Hz, 2H), 3.77 (s, 3H), 3.49-3.38 (m, 1H), 2.90 (t, J = 5.6 Hz, 2H), 1.17 (d, J = 6.8 Hz, 6H). LC-MS: m/z 482.3 (M + H)⁺ |
| 5. | I-36 | I-59 | 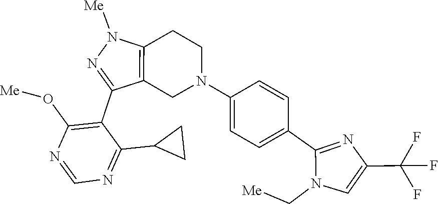 [SMILES: CCn1cc(C(F)(F)F)nc1-c1ccc(N2CCc3c(c(-c4c(OC)ncnc4C4CC4)nn3C)C2)cc1] | ¹H NMR (400 MHz, DMSO-d₆): δ 8.62 (s, 1H), 7.93 (s, 1H), 7.43 (d, J = 8.8 Hz, 2H), 7.08 (d, J = 8.8 Hz, 2H), 4.14 (s, 2H), 4.05 (q, J = 7.6 Hz, 2H), 3.91 (s, 3H), 3.77 (t, J = 5.6 Hz, 2H), 3.75 (s, 3H), 2.85 (t, J = 5.6 Hz, 2H), 2.18-2.12 (m, 1H), 1.31 (t, J = 6.8 Hz, 3H), 1.06-1.02 (m, 2H), 0.94-0.89 (m, 2H). LC-MS: m/z 524.1 (M + H)⁺ |
| 6. | I-36 | 1-60 | 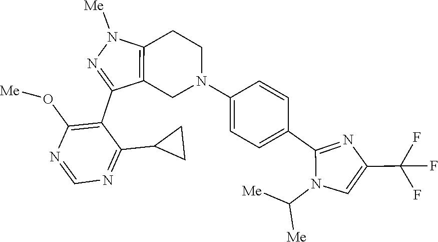 [SMILES: COc1ncnc(C2CC2)c1-c1nn(C)c2c1CN(c1ccc(-c3nc(C(F)(F)F)cn3C(C)C)cc1)CC2] | ¹H NMR (400 MHz, DMSO-d₆): δ 8.63 (s, 1H), 8.08 (d, J = 1.2 Hz, 1H), 7.37 (d, J = 9.2 Hz, 2H), 7.10 (d, J = 8.8 Hz, 2H), 4.51-4.42 (m, 1H), 4.15 (s, 2H), 3.92 (s, 3H), 3.78 (t, J = 5.6 Hz, 2H), 3.76 (s, 3H), 2.86 (t, J = 5.6 Hz, 2H), 2.18-2.12 (m, 1H), 1.39 (d, J = 6.4 Hz, 6H), 1.06-1.02 (m, 2H), 0.94-0.89 (m, 2H). LC-MS: m/z 538 (M + H)⁺ |
| 7. | I-36 | I-78 | 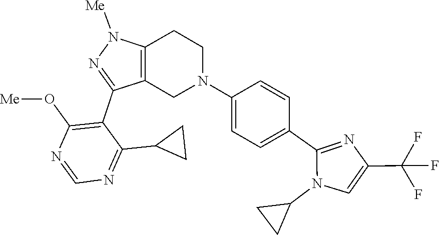 [SMILES: COc1ncnc(C2CC2)c1-c1nn(C)c2c1CN(c1ccc(-c3nc(C(F)(F)F)cn3C3CC3)cc1)CC2] | ¹H NMR (400 MHz, DMSO-d₆): δ 8.59 (s, 1H), 7.78 (s, 1H), 7.69 (d, J = 9.2 Hz, 2H), 7.03 (d, J = 8.8 Hz, 2H), 4.11 (s, 2H), 3.88 (s, 3H), 3.74 (t, J = 5.6 Hz, 2H), 3.72 (s, 3H), 3.68-3.62 (m, 1H), 2.83 (t, J = 5.6 Hz, 2H), 2.12- 2.08 (m, 1H), 1.02-0.98 (m, 2H), 0.94-0.85 (m, 6H). LC-MS: m/z 536.2 (M + H)⁺ |
| 8. | I-36 | I-61 | 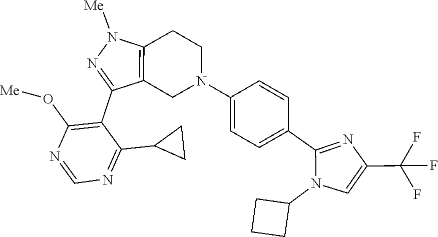 [SMILES: COc1ncnc(C2CC2)c1-c1nn(C)c2c1CN(c1ccc(-c3nc(C(F)(F)F)cn3C3CCC3)cc1)CC2] | ¹H NMR (400 MHz, DMSO-d₆): δ 8.62 (s, 1H), 8.19 (s, 1H), 7.36 (d, J = 8.8 Hz, 2H), 7.08 (d, J = 8.8 Hz, 2H), 4.76-4.62 (m, 1H), 4.14 (s, 2H), 3.91 (s, 3H), 3.77 (t, J = 5.6 Hz, 2H), 3.75 (s, 3H), 2.87 (t, J = 5.6 Hz., 2H), 2.45- 2.30 (m, 4H), 2.18-2.12 (m, 1H), 1.67-1.55 (m, 2H), 1.02-0.98 (m, 2H), 0.94-0.89 (m, 2H). LC-MS: m/z 550.2 (M + H)⁺ |
| 9. | I-36 | I-62 | 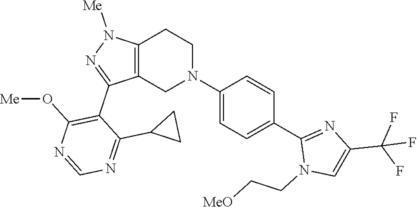 [SMILES: COCCn1cc(C(F)(F)F)nc1-c1ccc(N2CCc3c(c(-c4c(OC)ncnc4C4CC4)nn3C)C2)cc1] | ¹H NMR (400 MHz, DMSO-d₆): δ 8.62 (s, 1H), 7.88 (s, 1H), 7.47 (d, J = 8.8 Hz, 2H), 7.07 (d, J = 8.8 Hz, 2H), 4.17 (t, J = 5.0 Hz, 2H), 4.14 (s, 2H), 3.91 (s, 3H), 3.82 (t, J = 5.6 Hz, 2H), 3.77 (s, 3H), 3.64 (t, J = 4.8 Hz, 2H), 3.20 (s, 3H), 2.84 (t, J = 5.6 Hz, 2H), 2.18-2.12 (m, 1H), 1.06- 1.02 (m, 2H), 0.94-0.89 (m, 2H). LC-MS: m/z 554.1 (M + H)⁺ |
| 10. | I-36 | I-63 | 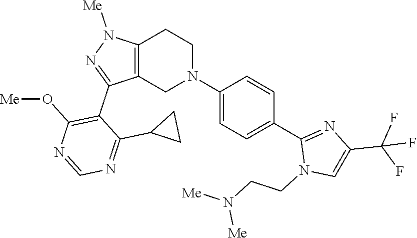 [SMILES: COc1ncnc(C2CC2)c1-c1nn(C)c2c1CN(c1ccc(-c3nc(C(F)(F)F)cn3CCN(C)C)cc1)CC2] | ¹H NMR (400 MHz, DMSO-d₆): δ 8.63 (s, 1H), 7.91 (d, J = 1.6 Hz, 1H), 7.45 (d, J = 8.8 Hz, 2H), 7.08 (d, J = 9.2 Hz, 2H), 4.14 (s, 2H), 4.10 (t, J = 6.0 Hz, 2H), 3.91 (s, 3H), 3.78 (t, J = 5.6 Hz, 2H), 3.75 (s, 3H), 2,84 (t, J = 5.6 Hz, 2H), 2.54 (t, J = 6.0 Hz, 2H), 2.18-2.12 (m, 1H), 2.08 (s, 6H), 1.06-1.02 (m, 2H), 0.94-0.89 (m, 2H). LC-MS: m/z 567.2 (M + H)⁺ |
| 11. | I-36 | I-94 | 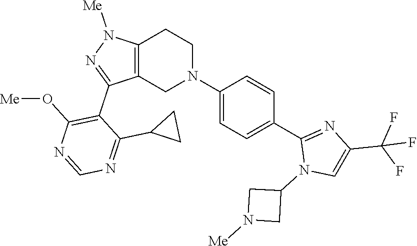 [SMILES: COc1ncnc(C2CC2)c1-c1nn(C)c2c1CN(c1ccc(-c3nc(C(F)(F)F)cn3C3CN(C)C3)cc1)CC2] | ¹H NMR (400 MHz, DMSO-d₆): δ 8.63 (s, 1H), 8.19 (s, 1H), 7.35 (d, J = 8.8 Hz, 2H), 7.08 (d, J = 9.2 Hz, 2H), 4.87-4.81 (m, 1H), 4.14 (s, 2H), 3.92 (s, 3H), 3.78 (t, J = 5.6 Hz, 2H), 3.76 (s, 3H), 3.59 (dd, J = 8.2, 7.2 Hz, 2H), 3.35 (dd, J = 8.2, 7.2 Hz, 2H), 2.85 (t, J = 5.6 Hz, 2H), 2.27 (s, 3H), 2.18-2.12 (m, 1H), 1.06- 1.02 (m, 2H), 0.94-0.89 (m, 2H). LC-MS: m/z 565.2 (M + H)⁺ |
| 12. | I-36 | I-65 | 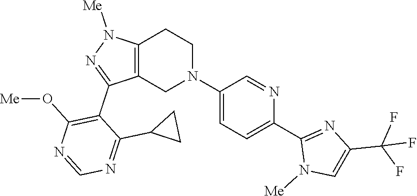 [SMILES: COc1ncnc(C2CC2)c1-c1nn(C)c2c1CN(c1ccc(-c3nc(C(F)(F)F)cn3C)nc1)CC2] | ¹H NMR (400 MHz, DMSO-d₆): δ 8.63 (s, 1H), 8.41 (d, J = 2.8 Hz, 1H), 7.87 (s, 1H), 7.86 (d, J = 8.8 Hz, 1H), 7.51 (dd, J = 9.0, 2.6 Hz., 1H), 4.21 (s, 2H), 4.02 (s, 3H), 3.92 (s, 3H), 3.77 (s, 3H), 3.83 (t, J = 5.6 Hz, 2H), 2.87 (t, J = 5.6 Hz, 2H), 2.18-2.12 (m, 1H), 1.07-1.02 (m, 2H), 0.95- 0.89 (m, 2H). LC-MS: m/z 511.3 (M + H)⁺ |
| 13. | I-36 | I-66 | 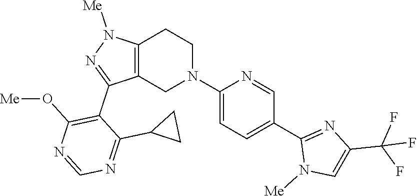 [SMILES: COc1ncnc(C2CC2)c1-c1nn(C)c2c1CN(c1ccc(-c3nc(C(F)(F)F)cn3C)cn1)CC2] | ¹H NMR (400 MHz, DMSO-d₆): δ 8.62 (s, 1H), 8.42 (d, J = 2.0 Hz, 1H), 7.87 (d, J = 1.2 Hz, 1H), 7.85 (dd, J = 8.8, 2.4 Hz, 1H), 7.04 (d, J = 8.8 Hz, 1H), 4.47 (s, 2H), 4.03 (t, J = 5.8 Hz, 2H), 3.91 (s, 3H), 3.76 (s, 3H), 3.75 (s, 3H), 2.87 (t, J = 5.6 Hz, 2H), 2.22-2.15 (m, 1H), 1.07-1.02 (m, 2H), 0.96-0.90 (m, 2H). LC-MS: m/z 511.1 (M + H)⁺ |
| 14. | I-36 | I-67 | 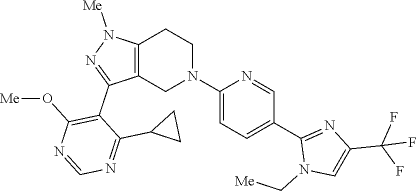 [SMILES: CCn1cc(C(F)(F)F)nc1-c1ccc(N2CCc3c(c(-c4c(OC)ncnc4C4CC4)nn3C)C2)nc1] | ¹H NMR (400 MHz, DMSO-d₆): 8.63 (s, 1H), 8.33 (d, J = 2.4 Hz, 1H), 7.98 (app. t, J = 1.2 Hz, 1H), 7.78 (dd, J = 8.8, 2.4 Hz, 1H), 7.04 (d, J = 8.4 Hz, 1H), 4.47 (s, 2H), 4.08-4.02 (m, 4H), 3.91 (s, 3H), 3.76 (s, 3H), 2.87 (t, J = 5.6 Hz, 2H), 2.22-2.15 (m, 1H), 1.32 (t, J = 7.2 Hz, 3H), 1.07-1.02 (m, 2H), 0.96-0.90 (m, 2H). LC-MS: m/z 525.3 (M + H)⁺ |
| 15. | I-36 | I-68 | 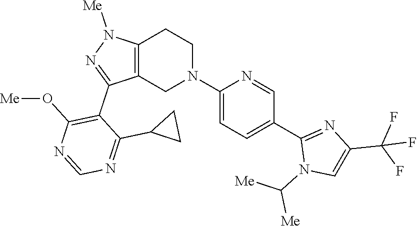 [SMILES: COc1ncnc(C2CC2)c1-c1nn(C)c2c1CN(c1ccc(-c3nc(C(F)(F)F)cn3C(C)C)cn1)CC2] | ¹H NMR (400 MHz, DMSO-d₆): 8.63 (s, 1H), 8.26 (d, J = 2.0 Hz, 1H), 8.13 (d, J = 1.2 Hz, 1H), 7.70 (dd, J = 8.8, 2.4 Hz, 1H), 7.04 (d, J = 9.2 Hz, 1H), 4.47 (s, 2H), 4.45-4.40 (m, 1H), 4.03 (t, J = 5.6 Hz, 2H), 3.91 (s, 3H), 3.76 (s, 3H), 2.87 (t, J = 5.6 Hz, 2H), 2.22-2.15 (m, 1H), 1.40 (d, J = 6.4 Hz, 6H), 1.07-1.02 (m, 2H), 0.96-0.90 (m, 2H); LC-MS: m/z 539.3 (M + H)⁺ |
| 16. | I-36 | I-69 | 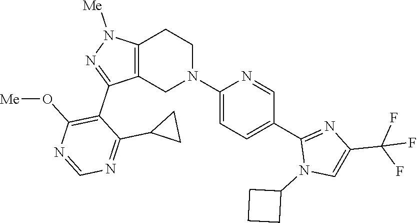 [SMILES: COc1ncnc(C2CC2)c1-c1nn(C)c2c1CN(c1ccc(-c3nc(C(F)(F)F)cn3C3CCC3)cn1)CC2] | ¹H NMR (400 MHz, DMSO-d₆): 8.63 (s, 1H), 8.25 (d, J = 2.0 Hz, 1H), 8.22 (d, J = 1.2 Hz, 1H), 7.70 (dd, J = 8.8, 2.4 Hz, 1H), 7.04 (d, J = 9.2 Hz, 1H), 4.78- 4.69 (m, 1H), 4.47 (s, 2H), 4.03 (t, J = 5.6 Hz, 2H), 3.91 (s, 3H), 3.76 (s, 3H), 2.87 (t, J = 5.6 Hz, 2H), 2.42-2.32 (m, 4H), 2.22- 2.15 (m, 1H), 1.80-1.68 (m, 2H), 1.07-1.02 (m, 2H), 0.96-0.90 (m, 2H); LC-MS: m/z 551.3 (M + H)⁺ |
| 17. | I-36 | I-80 | 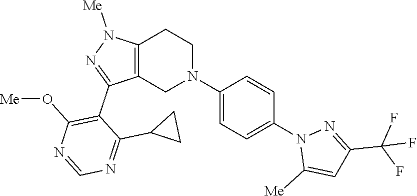 [SMILES: COc1ncnc(C2CC2)c1-c1nn(C)c2c1CN(c1ccc(-n3nc(C(F)(F)F)cc3C)cc1)CC2] | ¹H NMR (400 MHz, DMSO-d₆): δ 8.62 (s, 1H), 7.34 (d, J = 8.8 Hz, 2H), 7.10 (d, J = 8.4 Hz, 2H), 6.69 (s, 1H), 4.14 (s, 2H), 3.91 (s, 3H), 3.77 (t, J = 5.6 Hz, 2H), 3.76 (s, 3H), 2.86 (t, J = 5.6 Hz, 2H), 2.27 (s, 3H), 2.18-2.12 (m, 1H), 1.06-1.02 (m, 2H), 0.94- 0.89 (m, 2H). LC-MS: m/z 510.3 (M + H)⁺ |
| 18. | I-36 | I-79 | 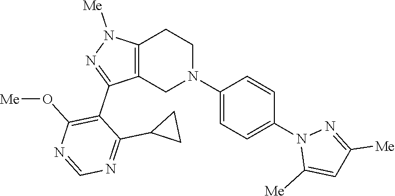 [SMILES: COc1ncnc(C2CC2)c1-c1nn(C)c2c1CN(c1ccc(-n3nc(C)cc3C)cc1)CC2] | ¹H NMR (400 MHz, DMSO-d₆): δ 8.62 (s, 1H), 7.25 (d, J = 9.2 Hz, 2H), 7.05 (d, J = 9.2 Hz, 2H), 5.99 (s, 1H), 4.09 (s, 2H), 3.91 (s, 3H), 3.76 (s, 3H), 3.73 (t, J = 5.6 Hz, 2H), 2.85 (t, J = 5.6 Hz, 2H), 2.20 (s, 3H), 2.14 (s, 3H), 2.18-2.12 (m, 1H), 1.06-1.02 (m, 2H), 0.94-0.89 (m, 2H). LC-MS: m/z 456.3 (M + H)⁺ |
| 19. | I-36 | I-83 | 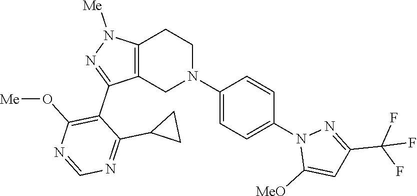 [SMILES: COc1ncnc(C2CC2)c1-c1nn(C)c2c1CN(c1ccc(-n3nc(C(F)(F)F)cc3OC)cc1)CC2] | ¹H NMR (400 MHz, DMSO-d₆): δ 8.62 (s, 1H), 7.38 (d, J = 8.8 Hz, 2H), 7.08 (d, J = 8.8 Hz, 2H), 6.39 (s, 1H), 4.11 (s, 2H), 3.95 (s, 3H), 3.91 (s, 3H), 3.75 (s, 3H), 3.74 (t, J = 5.6 Hz, 2H), 2.85 (t, J = 5.6 Hz, 2H), 2.18- 2.12 (m, 1H), 1.06-1.02 (m, 2H), 0.94-0.89 (m, 2H). LC-MS: m/z 526.3 (M + H)⁺ |
| 20. | I-36 | I-82 | 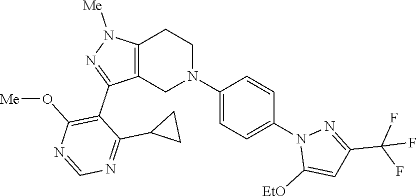 [SMILES: CCOc1cc(C(F)(F)F)nn1-c1ccc(N2CCc3c(c(-c4c(OC)ncnc4C4CC4)nn3C)C2)cc1] | ¹H NMR (400 MHz, DMSO-d₆): δ 8.62 (s, 1H), 7.41 (d, J = 9.2 Hz, 2H), 7.09 (d, J = 9.2 Hz, 2H), 6.38 (s, 1H), 4.24 (q, J = 6.8 Hz, 2H), 4.11 (s, 2H), 3.91 (s, 3H), 3.75 (s, 3H), 3.74 (t, J = 5.6 Hz, 2H), 2.85 (t, J = 5.6 Hz, 2H), 2.18-2.12 (m, 1H), 1.33 (t, J = 7.2 Hz, 3H), 1.06-1.02 (m, 2H), 0.94-0.89 (m, 2H). LC-MS: m/z 540.3 (M + H)⁺ |
| 21. | I-36 | I-91 | 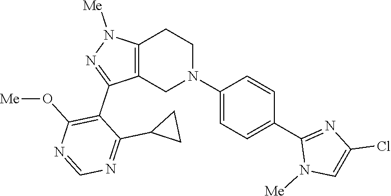 [SMILES: COc1ncnc(C2CC2)c1-c1nn(C)c2c1CN(c1ccc(-c3nc(Cl)cn3C)cc1)CC2] | ¹H NMR (400 MHz, DMSO-d₆): δ 8.62 (s, 1H), 7.48 (d, J = 8.8 Hz, 2H), 7.27 (s, 1H), 7.06 (d, J = 9.2 Hz, 2H), 4.13 (s, 2H), 3.92 (s, 3H), 3.89-3.82 (m, 5H), 3.73 (s, 3H), 2.85 (t, J = 5.6 Hz, 2H), 2.18-2.12 (m, 1H), 1.06-1.02 (m, 2H), 0.94-0.89 (m, 2H). LC-MS: m/z 476.3 (M + H)⁺ |
| 22. | I-36 | I-84 | 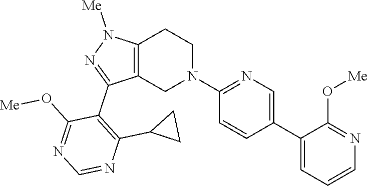 [SMILES: COc1ncccc1-c1ccc(N2CCc3c(c(-c4c(OC)ncnc4C4CC4)nn3C)C2)nc1] | ¹H NMR (400 MHz, DMSO-d₆): δ 8.63 (s, 1H), 8.29 (d, J = 2.8 Hz, 1H), 8.12 (dd, J = 4.8, 2.0 Hz, 1H), 7.74 (app. td, J = 8.0, 2.0 Hz, 2H), 7.06 (dd, J = 7.2, 4.8 Hz, 1H), 6.90 (d, J = 8.8 Hz, 1H), 4.43 (s, 2H), 3.99 (t, J = 5.8 Hz, 2H), 3.91 (s, 3H), 3.87 (s, 3H), 3.75 (s, 3H), 2.85 (t, J = 5.6 Hz, 2H), 2.22-2.15 (m, 1H), 1.07-1.02 (m, 2H), 0.96-0.90 (m, 2H). LC-MS: m/z 470.3 (M + H)⁺ |
| 23. | I-36 | I-85 | 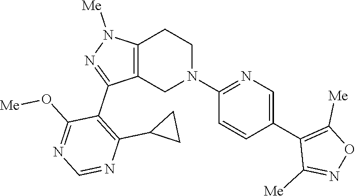 [SMILES: COc1ncnc(C2CC2)c1-c1nn(C)c2c1CN(c1ccc(-c3c(C)noc3C)cn1)CC2] | ¹H NMR (400 MHz, DMSO-d₆): δ 8.63 (s, 1H), 8.10 (d, J = 2.4 Hz, 1H), 7.56 (dd, J = 9.0, 2.6 Hz, 1H), 7.01 (d, J = 9.2 Hz, 1H), 4.42 (s, 2H), 3.99 (t, J = 5.8 Hz, 2H), 3.91 (s, 3H), 3.76 (s, 3H), 2.86 (t, J = 5.6 Hz, 2H), 2.36 (s, 3H), 2.19 (s, 3H), 2.19-2.15 (m, 1H), 1.06-1.02 (m, 2H), 0.95- 0.90 (m, 2H). LC-MS: m/z 458.3 (M + H)⁺ |
| 24. | I-37 | 1-58 | 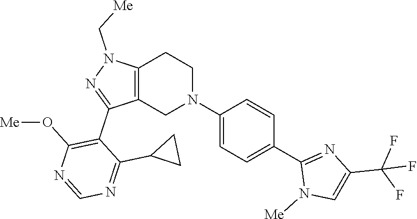 [SMILES: CCn1nc(-c2c(OC)ncnc2C2CC2)c2c1CCN(c1ccc(-c3nc(C(F)(F)F)cn3C)cc1)C2] | ¹H NMR (400 MHz, DMSO-d₆): δ 8.59 (s, 1H), 7.80 (s, 1H), 7.49 (d, J = 8.0 Hz, 2H), 7.05 (d, J = 8.0 Hz, 2H), 4.11 (s, 2H), 4.03 (q, J = 7.2 Hz, 2H), 3.89 (s, 3H), 3.73 (t, J = 5.6 Hz, 2H), 3.70 (s, 3H), 2.84 (t. J = 5.6 Hz, 2H), 2.18-2.12 (m, 1H), 1.30 (t, J = 7.2 Hz, 3H), 1.04-0.98 (m, 2H), 0.90-0.82 (m, 2H). LC-MS: m/z 524.1 (M + H)⁺ |
| 25. | I-37 | I-65 | 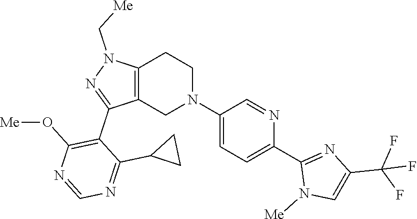 [SMILES: CCn1nc(-c2c(OC)ncnc2C2CC2)c2c1CCN(c1ccc(-c3nc(C(F)(F)F)cn3C)nc1)C2] | ¹H NMR (400 MHz, DMSO-d₆): δ 8.62 (s, 1H), 8.42 (d, J = 2.8 Hz, 1H), 7.88 (s, 1H), 7.86 (d, J = 8.4 Hz, 1H), 7.51 (dd, J = 8.8, 2.8 Hz, 1H), 4.21 (s, 2H), 4.07 (q, J = 7.2 Hz, 2H), 4.02 (s, 3H), 3.93 (s, 3H), 3.83 (t, J = 5.8 Hz, 2H), 2.90 (t, J = 5.6 Hz, 2H), 2.22-2.15 (m, 1H), 1.34 (t, J = 7.2 Hz, 3H), 1.07-1.02 (m, 2H), 0.96-0.90 (m, 2H). LC-MS: m/z 525.4 (M + H)⁺ |
| 26. | I-37 | I-66 | 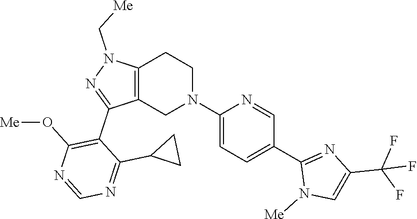 [SMILES: CCn1nc(-c2c(OC)ncnc2C2CC2)c2c1CCN(c1ccc(-c3nc(C(F)(F)F)cn3C)cn1)C2] | ¹H NMR (400 MHz, DMSO-d₆): δ 8.63 (s, 1H), 8.42 (d, J = 2.0 Hz, 1H), 7.88 (d, J = 1.2 Hz, 1H), 7.85 (dd, J = 8.8, 2.4 Hz, 1H), 7.03 (d, J = 9.2 Hz, 1H), 4.47 (s, 2H), 4.07 (q, J = 7.2 Hz, 2H), 4.04 (t, J = 5.8 Hz, 2H), 3.93 (s, 3H), 3.75 (s, 3H), 2.89 (t, J = 5.6 Hz, 2H), 2.22-2.15 (m, 1H), 1.34 (t, J = 7.2 Hz, 3H), 1.07-1.02 (m, 2H), 0.96-0.90 (m, 2H). LC-MS: m/z 525.4 (M + H)⁺ |
| 27. | I-38 | I-58 | 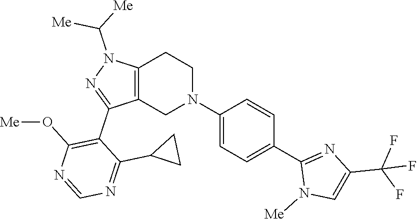 [SMILES: COc1ncnc(C2CC2)c1-c1nn(C(C)C)c2c1CN(c1ccc(-c3nc(C(F)(F)F)cn3C)cc1)CC2] | ¹H NMR (400 MHz, DMSO-d₆): δ 8.58 (s, 1H), 7.80 (d, J = 0.8 Hz, 1H), 7.50 (d, J = 8.8 Hz, 2H), 7.05 (d, J = 8.8 Hz, 2H), 4.50- 4.42 (m, 1H), 4.11 (s, 2H), 3.90 (s, 3H), 3.73 (t, J = 5.6 Hz, 2H), 3.70 (s, 3H), 2.86 (t, J = 5.6 Hz, 2H), 2.18-2.12 (m, 1H), 1.36 (d, J = 6.8 Hz, 6H), 1.02-0.98 (m, 2H), 0.90-0.82 (m, 2H). LC-MS: m/z 538.3 (M + H)⁺ |
| 28. | I-38 | I-65 | 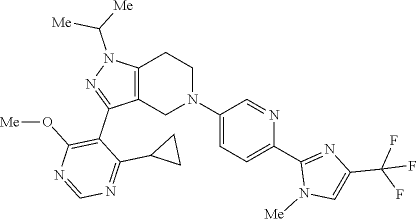 [SMILES: COc1ncnc(C2CC2)c1-c1nn(C(C)C)c2c1CN(c1ccc(-c3nc(C(F)(F)F)cn3C)nc1)CC2] | ¹H NMR (400 MHz, DMSO-d₆): δ 8.59 (s, 1H), 8.38 (d, J = 3.2 Hz, 1H), 7.84 (s, 1H), 7.83 (d, J = 8.8 Hz, 1H), 7.47 (dd, J = 9.2, 3.2 Hz, 1H), 4.50-4.42 (m, 1H), 4.17 (s, 2H), 3.99 (s, 3H), 3.90 (s, 3H), 3.79 (t, J = 5.8 Hz, 2H), 2.89 (t, J = 5.6 Hz, 2H), 2.22- 2.15 (m, 1H), 1.35 (d, J = 6.8 Hz, 6H), 1.02-0.96 (m, 2H), 0.90- 0.85 (m, 2H). LC-MS: m/z 539.3 (M + H)⁺ |
| 29. | I-38 | I-66 | 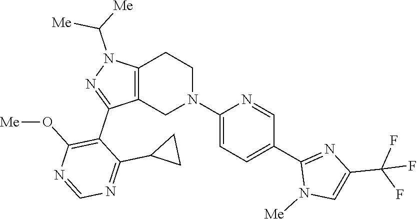 [SMILES: COc1ncnc(C2CC2)c1-c1nn(C(C)C)c2c1CN(c1ccc(-c3nc(C(F)(F)F)cn3C)cn1)CC2] | ¹H NMR (400 MHz, DMSO-d₆): δ 8.59 (s, 1H), 8.38 (d, J = 3.2 Hz, 1H), 7.83 (d, J = 2.2 Hz, 1H), 7.81 (dd, J = 9.2, 2.4 Hz, 1H), 7.00 (d, J = 9.2 Hz, 1H), 4.50- 4.42 (m, 1H), 4.43 (s, 2H), 4.02 (t, J = 5.8 Hz, 2H), 3.99 (s, 3H), 3.71 (s, 3H), 2.86 (t, J = 5.6 Hz, 2H), 2.22-2.15 (m, 1H), 1.36 (d, J = 6.8 Hz, 6H), 1.02-0.96 (m, 2H), 0.90-0.85 (m, 2H). LC-MS: m/z 539.3 (M + H)⁺ |
| 30. | I-39 | I-58 | 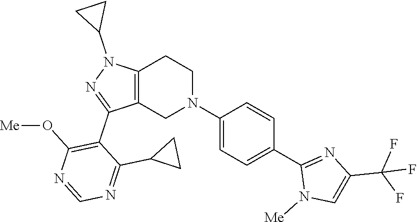 [SMILES: COc1ncnc(C2CC2)c1-c1nn(C2CC2)c2c1CN(c1ccc(-c3nc(C(F)(F)F)cn3C)cc1)CC2] | ¹H NMR (400 MHz, DMSO-d₆): δ 8.62 (s, 1H), 7.84 (d, J = 1.2 Hz, 1H), 7.54 (d, J = 8.8 Hz, 2H), 7.09 (d, J = 9.2 Hz, 2H), 4.14 (s, 2H), 3.92 (s, 3H), 3.77 (t, J = 5.6 Hz, 2H), 3.74 (s, 3H), 3.60-3.53 (m, 1H), 2.96 (t, J = 5.6 Hz, 2H), 2.18-2.12 (m, 1H), 1.08-1.00 (m, 4H), 0.99-0.89 (m, 4H). LC-MS: m/z 536.1 (M + H)⁺ |
| 31. | I-39 | I-65 | 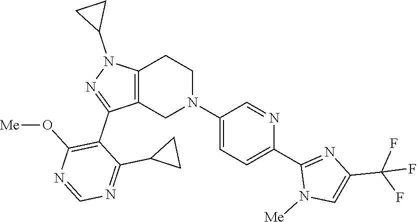 [SMILES: COc1ncnc(C2CC2)c1-c1nn(C2CC2)c2c1CN(c1ccc(-c3nc(C(F)(F)F)cn3C)nc1)CC2] | ¹H NMR (400 MHz, DMSO-d₆): δ 8.62 (s, 1H), 8.42 (d, J = 2.8 Hz, 1H), 7.88 (s, 1H), 7.87 (d, J = 8.4 Hz, 1H), 7.52 (dd, J = 8.8, 3.0 Hz, 1H), 4.20 (s, 2H), 4.03 (s, 3H), 3.93 (s, 3H), 3.84 (t, J = 5.6 Hz, 2H), 3.58-3.52 (m, 1H), 2.97 (t, J = 5.4 Hz, 2H), 2.18-2.12 (m, 1H), 1.06-0.90 (m, 8H). LC-MS: m/z 537.1 (M + H)⁺ |
| 32. | I-39 | I-66 | 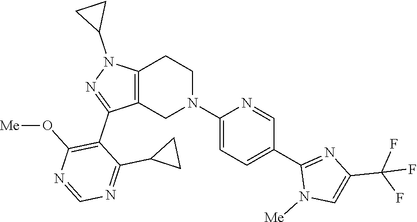 [SMILES: COc1ncnc(C2CC2)c1-c1nn(C2CC2)c2c1CN(c1ccc(-c3nc(C(F)(F)F)cn3C)cn1)CC2] | ¹H NMR (400 MHz, DMSO-d₆): δ 8.62 (s, 1H), 8.42 (d, J = 2.0 Hz, 1H), 7.88 (d, J = 1.2 Hz, 1H), 7.85 (dd, J = 8.8, 2.4 Hz, 1H), 7.03 (d, J = 8.8 Hz, 1H), 4.46 (s, 2H), 4.05 (t, J = 5.6 Hz, 2H), 3.92 (s, 3H), 3.75 (s, 3H), 3.58- 3.52 (m, 1H), 2.96 (t, J = 5.6 Hz, 2H), 2.18-2.12 (m, 1H), 1.06- 0.90 (m, 8H). LC-MS: m/z 537.1 (M + H)⁺ |
| 33. | I-40 | I-58 | 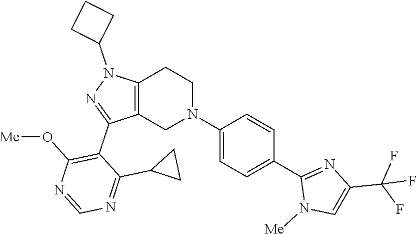 [SMILES: COc1ncnc(C2CC2)c1-c1nn(C2CCC2)c2c1CN(c1ccc(-c3nc(C(F)(F)F)cn3C)cc1)CC2] | ¹H NMR (400 MHz, DMSO-d₆): δ 8.60 (s, 1H), 7.80 (s, 1H), 7.49 (d, J = 8.8 Hz, 2H), 7.04 (d, J = 8.8 Hz, 2H), 4.78-4.72 (m, 1H), 4.11 (s, 2H), 3.89 (s, 3H), 3.72 (t, J = 5.6 H7, 2H), 3.70 (s, 3H), 2.82 (t, J = 5.4 Hz, 2H), 2.55- 2.50 (m, 1H, merged in DMSO- d₆), 2.35-2.28 (m, 3H), 2.18-2.12 (m, 1H), 1.79-1.60 (m, 2H), 1.02-0.98 (m, 2H), 0.92-0.86 (m, 2H). LC-MS: m/z 550.3 (M + H)⁺ |
| 34. | I-40 | I-65 | 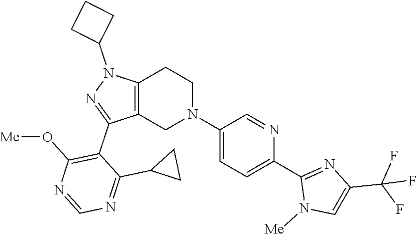 [SMILES: COc1ncnc(C2CC2)c1-c1nn(C2CCC2)c2c1CN(c1ccc(-c3nc(C(F)(F)F)cn3C)nc1)CC2] | ¹H NMR (400 MHz, DMSO-d₆): δ 8.64 (s, 1H), 8.41 (d, J = 3.2 Hz, 1H), 7.87 (s, 1H), 7.86 (d, J = 8.8 Hz, 1H), 7.51 (dd, J = 9.0, 2.8 Hz, 1H), 4.82-4.75 (m, 1H), 4.21 (s, 2H), 4.02 (s, 3H), 3.94 (s, 3H), 3.82 (t, J = 5.6 Hz, 2H), 2.87 (t, J = 5.6 Hz, 2H), 2.60- 2.52 (m, 2H), 2.39-2.30 (m, 2H), 2.22-2.15 (m, 1H), 1.85-1.72 (m, 2H), 1.06-1.02 (m, 2H), 0.97- 0.92 (m, 2H). LC-MS: m/z 551.2 (M + H)⁺ |
| 35. | I-40 | I-66 | 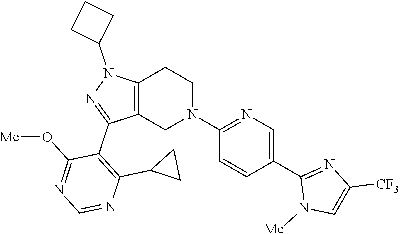 [SMILES: COc1ncnc(C2CC2)c1-c1nn(C2CCC2)c2c1CN(c1ccc(-c3nc(C(F)(F)F)cn3C)cn1)CC2] | ¹H NMR (400 MHz, DMSO-d₆): δ 8.64 (s, 1H), 8.41 (d, J = 3.2 Hz, 1H), 7.87 (d, J = 1.2 Hz, 1H), 7.84 (dd, J = 8.8, 2.4 Hz, 1H), 7.02 (d, J = 8.8 Hz, 1H), 4.82- 4.75 (m, 1H), 4.47 (s, 2H), 4.02 (t, J = 5.6 Hz, 2H), 3.93 (s, 3H), 3.74 (s, 3H), 2.86 (t, J = 5.6 Hz, 2H), 2.59-2.52 (m, 2H), 2.39- 2.30 (m, 2H), 2.22-2.15 (m, 1H), 1.85-1.72 (m, 2H), 1.06-1.02 (m, 2H), 0.97-0.92 (m, 2H). LC-MS: m/z 551.2 (M + H)⁺ |
| 36. | I-41 | I-58 | 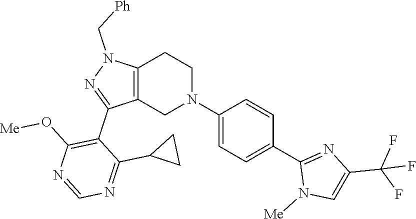 [SMILES: COc1ncnc(C2CC2)c1-c1nn(Cc2ccccc2)c2c1CN(c1ccc(-c3nc(C(F)(F)F)cn3C)cc1)CC2] | ¹H NMR (400 MHz, DMSO-d₆): δ 8.64 (s, 1H), 7.85 (d, J = 1.2 Hz, 1H), 7.52 (d, J = 8.8 Hz, 2H), 7.37-7.32 (m, 2H), 7.29- 7.25 (m, 1H), 7.23-7.19 (m, 2H), 7.07 (d, J = 8.8 Hz, 2H), 5.33 (s, 2H), 4.15 (s, 2H), 3.94 (s, 3H), 3.75 (t, J = 5.6 Hz, 2H), 3.74 (s, 3H), 2.68 (t, J = 5.6 Hz, 2H), 2.18-2.12 (m, 1H), 1.07-1.02 (m, 2H), 0.95-0.85 (m, 2H). LC-MS: m/z 586.2 (M + H)⁺ |
| 37. | I-41 | I-66 | 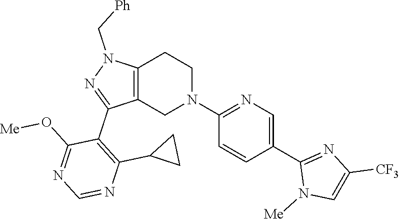 [SMILES: COc1ncnc(C2CC2)c1-c1nn(Cc2ccccc2)c2c1CN(c1ccc(-c3nc(C(F)(F)F)cn3C)cn1)CC2] | ¹H NMR (400 MHz, DMSO-d₆): δ 8.64 (s, 1H), 8.41 (d, J = 2.4 Hz, 1H), 7.88 (d, J = 1.2 Hz, 1H), 7.84 (dd, J = 9.0, 2.6 Hz, 1H), 7.38-7.22 (m, 5H), 7.00 (d, J = 9.0 Hz, 1H), 5.33 (s, 2H), 4.47 (s, 2H), 4.02 (t, J = 5.8 Hz, 2H), 3.93 (s, 3H), 3.74 (s, 3H), 2.82 (t, J = 5.6 Hz, 2H), 2.21-2.15 (m, 1H), 1.07-1.02 (m, 2H), 0.96- 0.89 (m, 2H). LC-MS: m/z 587.2 (M + H)⁺ |
| 38. | I-42 | I-58 | 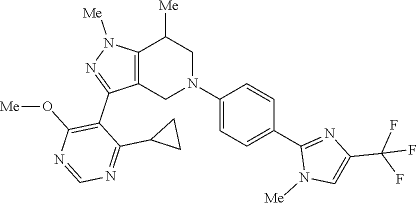 [SMILES: COc1ncnc(C2CC2)c1-c1nn(C)c2c1CN(c1ccc(-c3nc(C(F)(F)F)cn3C)cc1)CC2C] | ¹H NMR (400 MHz, DMSO-d₆): δ 8.63 (s, 1H), 7.84 (d, J = 1.2 Hz, 1H), 7.54 (d, J = 9.2 Hz, 2H), 7.07 (d, J = 9.2 Hz, 2H), 4.16 (d, J = 14.4 Hz, 1H), 3.92 (d, J = 14.4 Hz, 1H), 3.91 (s, 3H), 3.79- 3.73 (m, 4H), 3.74 (s, 3H), 3.49 (dd, J = 13.2, 4.0 Hz, 1H), 3.29- 3.20 (m, 1H), 2.15-2.09 (m, 1H), 1.35 (d, J = 6.8 Hz, 3H), 1.05- 1.00 (m, 2H), 0.97-0.90 (m, 2H). LC-MS: m/z 524.1 (M + H)⁺ |
| 39. | I-42 | I-65 | 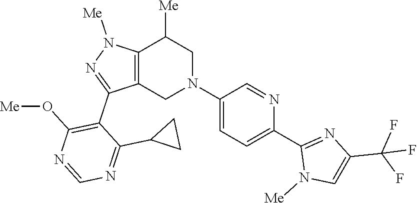 [SMILES: COc1ncnc(C2CC2)c1-c1nn(C)c2c1CN(c1ccc(-c3nc(C(F)(F)F)cn3C)nc1)CC2C] | ¹H NMR (400 MHz, DMSO-d₆): δ 8.59 (s, 1H), 8.37 (d, J = 2.4 Hz, 1H), 7.84 (s, 1H), 7.82 (d, J = 8.8 Hz, 1H), 7.46 (dd, J = 8.8, 2.4 Hz, 1H), 4.19 (d, J = 14.4 Hz, 1H), 3.99 (s, 3H), 3.95 (d, J = 14.4 Hz, 1H), 3.87 (s, 3H), 3.80 (s, 3H), 3.79 (dd, J = 13.2, 4.0 Hz, 1H), 3.50 (dd, J = 13.2, 4.0 Hz, 1H), 3.29-3.21 (m, 1H), 2.11-2.05 (m, 1H), 1.31 (d, J = 6.8 Hz, 3H), 1.05-0.99 (m, 2H), 0.92-0.85 (m, 2H). |
| | | | | LC-MS: m/z 525.1 (M + H)⁺ |
| 40. | I-42 | I-66 | 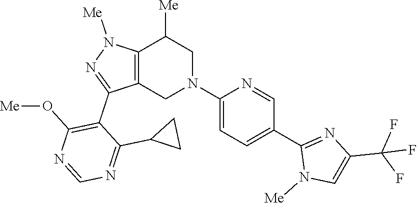 [SMILES: COc1ncnc(C2CC2)c1-c1nn(C)c2c1CN(c1ccc(-c3nc(C(F)(F)F)cn3C)cn1)CC2C] | ¹H NMR (400 MHz, DMSO-d₆): δ 8.59 (s, 1H), 8.37 (d, J = 2.0 Hz, 1H), 7.85 (s, 1H), 7.80 (dd, J = 9.0, 2.6 Hz, 1H), 7.00 (d, J = 8.8 Hz, 1H), 4.61 (d, J = 15.6 Hz, 1H), 4.25 (dd, J = 13.2, 3.0 Hz, 1H), 4.07 (d, J = 15.6 Hz, 1H), 3.87 (s, 3H), 3.80 (s, 3H), 3.71 (s, 3H), 3.57 (dd, J = 13.2, 3.8 Hz, 1H), 3.28-3.21 (m, 1H), 2.13-2.08 (m, 1H), 1.25 (d, J = 6.8 Hz, 3H), 1.05-0.90 (m, 2H), 0.92-0.85 (m, 2H). |
| | | | | LC-MS: m/z 525.1 (M + H)⁺ |
| 41. | I-43 | I-58 | 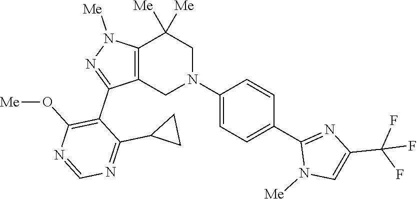 [SMILES: COc1ncnc(C2CC2)c1-c1nn(C)c2c1CN(c1ccc(-c3nc(C(F)(F)F)cn3C)cc1)CC2(C)C] | ¹H NMR (400 MHz, DMSO-d₆): δ 8.63 (s, 1H), 7.84 (d, J = 1.2 Hz 1H), 7.53 (d, J = 9.2 Hz, 2H), 7.06 (d, J = 8.8 Hz, 2H), 3.99 (s, 2H), 3.94 (s, 3H), 3.91 (s, 3H), 3.74 (s, 3H), 3.43 (s, 2H), 2.12- 2.06 (m, 1H), 1.44 (s, 6H), 1.06- 1.02 (m, 2H), 0.97-0.91 (m, 2H). LC-MS: m/z 538.1 (M + H)⁺ |
| 42. | I-43 | I-65 | 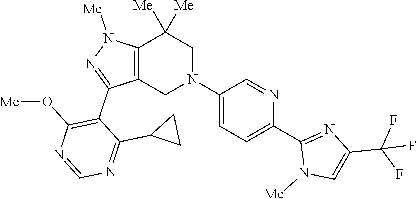 [SMILES: COc1ncnc(C2CC2)c1-c1nn(C)c2c1CN(c1ccc(-c3nc(C(F)(F)F)cn3C)nc1)CC2(C)C] | ¹H NMR (400 MHz, DMSO-d₆): δ 8.63 (s, 1H), 8.41 (d, J = 3.2 Hz, 1H), 7.87 (s, 1H), 7.86 (d, J = 8.8 Hz, 1H), 7.49 (dd, J = 8.8, 3.2 Hz, 1H), 4.06 (s, 2H), 4.03 (s, 3H), 3.94 (s, 3H), 3.91 (s, 3H), 3,49 (s, 2H), 2.12-2.08 (m, 1H), 1.44 (s, 6H), 1.07-1.02 (m, 2H), 0.95-0.90 (m, 2H). LC-MS: m/z 539.1 (M + H)⁺ |
| 43. | I-43 | I-66 | 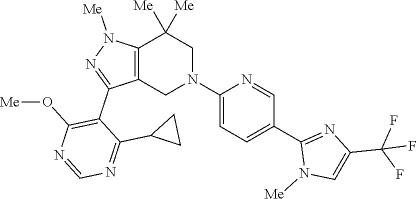 [SMILES: COc1ncnc(C2CC2)c1-c1nn(C)c2c1CN(c1ccc(-c3nc(C(F)(F)F)cn3C)cn1)CC2(C)C] | ¹H NMR (400 MHz, DMSO-d₆): δ 8.63 (s, 1H), 8.40 (d, J = 2.0 Hz, 1H), 7.88 (d, J = 1.2 Hz, 1H), 7.84 (dd, J = 9.0, 2.6 Hz, 1H), 7.05 (d, J = 9.2 Hz, 1H), 4.36 (s, 2H), 3.95 (s, 3H), 3.91 (s, 3H), 3.77 (s, 2H), 3.75 (s, 3H), 2.13- 2.08 (m, 1H), 1.44 (s, 6H), 1.07- 1.02 (m, 2H), 0.96-0.90 (m, 2H). LC-MS: m/z 539.2 (M + H)⁺ |
| 44. | I-38 | I-73 | 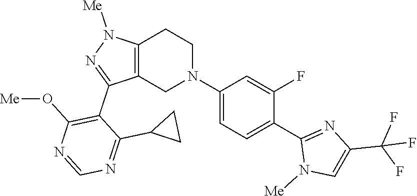 [SMILES: COc1ncnc(C2CC2)c1-c1nn(C)c2c1CN(c1ccc(-c3nc(C(F)(F)F)cn3C)c(F)c1)CC2] | ¹H NMR (400 MHz, DMSO-d₆): δ 8.62 (s, 1H), 7.91 (d, J = 0.8 Hz, 1H), 7.33 (t, J = 8.8 Hz, 1H), 7.00-6.90 (m, 2H), 4.18 (s, 2H), 3.91 (s, 3H), 3.81 (t, J = 5.6 Hz, 2H), 3.74 (s, 3H), 3.56 (s, 3H), 2.85 (t, J = 5.6 Hz, 2H), 2.18- 2.12 (m, 1H), 1.06-1.02 (m, 2H), 0.94-0.89 (m, 2H). LC-MS: m/z 528.3 (M + H)⁺ |
| 45. | I-38 | I-74 | 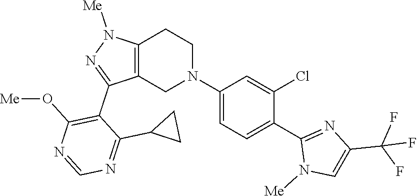 [SMILES: COc1ncnc(C2CC2)c1-c1nn(C)c2c1CN(c1ccc(-c3nc(C(F)(F)F)cn3C)c(Cl)c1)CC2] | ¹H NMR (400 MHz, DMSO-d₆): δ 8.62 (s, 1H), 7.91 (d, J = 1.2 Hz, 1H), 7.30 (d, J = 8.8 Hz, 1H), 7.17 (d, J = 3.2 Hz, 1H), 7.06 (dd, J = 8.8, 2.4 Hz, 1H), 4.18 (s, 2H), 3.91 (s, 3H), 3.80 (t, J = 5.6 Hz, 2H), 3.76 (s, 3H), 3.50 (s, 3H), 2.86 (t, J = 5.6 Hz, 2H), 2.18-2.12 (m, 1H), 1.06-1.02 (m, 2H), 0.94-0.89 (m, 2H). LC-MS: m/z 544.3 (M + H)⁺ |
| 46. | I-38 | I-70 | 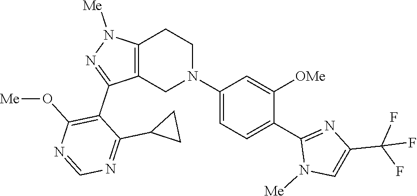 [SMILES: COc1cc(N2CCc3c(c(-c4c(OC)ncnc4C4CC4)nn3C)C2)ccc1-c1nc(C(F)(F)F)cn1C] | ¹H NMR (400 MHz, DMSO-d₆): δ 8.62 (s, 1H), 7.82 (d, J = 1.2 Hz, 1H), 7.13 (d, J = 8.4 Hz, 1H), 6.66 (d, J = 2.0 Hz, 1H), 6.62 (dd, J = 8.6, 2.2 Hz, 1H), 4.16 (s, 2H), 3.90 (s, 3H), 3.81 (t, J = 5.6 Hz, 2H), 3.79 (s, 3H), 3.76 (s, 3H), 3.45 (s, 3H), 2.88 (t, J = 5.6 Hz, 2H), 2.18-2.12 (m, 1H), 1.06-1.02 (m, 2H), 0.93-0.89 (m, 2H). LC-MS: m/z 540.4 (M + H)⁺ |
| 47. | I-38 | I-71 | 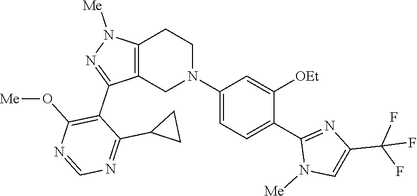 [SMILES: CCOc1cc(N2CCc3c(c(-c4c(OC)ncnc4C4CC4)nn3C)C2)ccc1-c1nc(C(F)(F)F)cn1C] | ¹H NMR (400 MHz, DMSO-d₆): δ 8.62 (s, 1H), 7.82 (d, J = 1.2 Hz, 1H), 7.14 (d, J = 8.4 Hz, 1H), 6.64 (d, J = 2.0 Hz, 1H), 6.62 (dd, J = 8.6, 2.2 Hz, 1H), 4.16 (s, 2H), 4.09 (q, J = 7.2 Hz, 2H), 3.90 (s, 3H), 3.81 (t, J = 5.6 Hz, 2H), 3.76 (s, 3H), 3.48 (s, 3H), 2.87 (t, J = 5.6 Hz, 2H), 2.18- 2.12 (m, 1H), 1.24 (t, J = 7.2 Hz, 3H), 1.06-1.02 (m, 2H), 0.94- 0.90 (m, 2H). LC-MS: m/z 554.4 (M + H)⁺ |
| 48. | I-38 | I-72 | 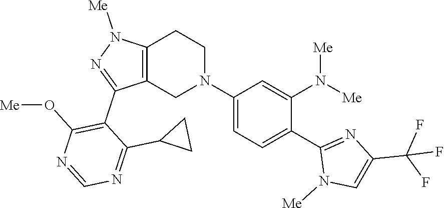 [SMILES: COc1ncnc(C2CC2)c1-c1nn(C)c2c1CN(c1ccc(-c3nc(C(F)(F)F)cn3C)c(N(C)C)c1)CC2] | ¹H NMR (400 MHz, DMSO-d₆): δ 8.62 (s, 1H), 7.83 (d, J = 1.2 Hz, 1H), 7.07 (d, J = 8.4 Hz, 1H), 6.60 (dd, J = 8.4, 2.4 Hz, 1H), 6.53 (dd, J = 2.4 Hz, 1H), 4.12 (s, 2H), 3.90 (s, 3H), 3.77 (s, 3H), 3.75 (t, J = 5.6 Hz, 2H), 3.43 (s, 3H), 2.86 (t, J = 5.6 Hz, 2H), 2.47 (s, 6H), 2.18-2.12 (m, 1H), 1.06-1.02 (m, 2H), 0.94- 0.89 (m, 2H). LC-MS: m/z 553.4 (M + H)⁺ |
| 49. | I-38 | I-76 | 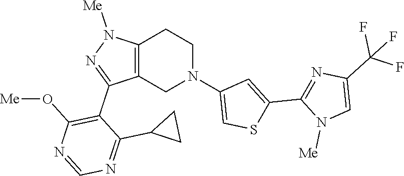 [SMILES: COc1ncnc(C2CC2)c1-c1nn(C)c2c1CN(c1csc(-c3nc(C(F)(F)F)cn3C)c1)CC2] | ¹H NMR (400 MHz, DMSO-d₆): δ 8.61 (s, 1H), 7.91 (d, J = 1.2 Hz, 1H), 7.47 (d, J = 1.6 Hz, 1H), 6.58 (d, J = 1.6 Hz, 1H), 3.96 (s, 2H), 3.89 (s, 3H), 3.87 (s, 3H), 3.77 (s, 3H), 3.59 (t, J = 5.6 Hz, 2H), 2.87 (t, J = 5.6 Hz, 2H), 2.18-2.12 (m, 1H), 1.05-1.01 (m, 2H), 0.95-0.90 (m, 2H). LC-MS: m/z 515.9 (M + H)⁺ |
| 50. | I-38 | I-77 | 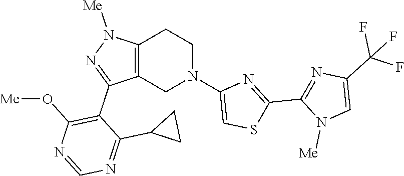 [SMILES: COc1ncnc(C2CC2)c1-c1nn(C)c2c1CN(c1csc(-c3nc(C(F)(F)F)cn3C)n1)CC2] | ¹H NMR (400 MHz, DMSO-d₆): δ 8.62 (s, 1H), 8.04 (d, J = 1.2 Hz, 1H), 6.53 (s, 1H), 4.13 (s, 2H), 4.04 (s, 3H), 3.89 (s, 3H), 3.82 (t, J = 5.6 Hz, 2H), 3.77 (s, 3H), 2.89 (t, J = 5.6 Hz, 2H), 2.16-2.12 (m, 1H), 1.05-1.01 (m, 2H), 0.94-0.89 (m, 2H). LC-MS: m/z 517.2 (M + H)⁺ |
| 51. | I-38 | I-88 | 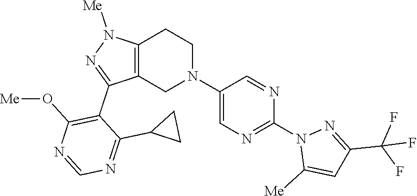 [SMILES: COc1ncnc(C2CC2)c1-c1nn(C)c2c1CN(c1cnc(-n3nc(C(F)(F)F)cc3C)nc1)CC2] | ¹H NMR (400 MHz, DMSO-d₆): δ 8.68 (s, 2H), 8.63 (s, 1H), 6.74 (s, 1H), 4.27 (s, 2H), 3.92 (s, 3H), 3.88 (t. J = 5.6 Hz, 2H), 3.76 (s, 3H), 2.91 (t, J = 5.6 Hz, 2H), 2.48 (s, 3H), 2.18-2.12 (m, 1H), 1.03-1.02 (m, 2H), 0.95- 0.89 (m, 2H). LC-MS: m/z 512.2 (M + H)⁺ |
| 52. | I-38 | I-87 | 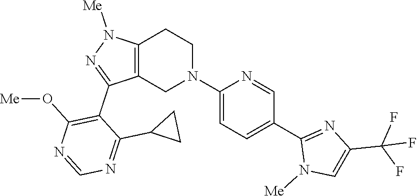 [SMILES: COc1ncnc(C2CC2)c1-c1nn(C)c2c1CN(c1ccc(-c3nc(C(F)(F)F)cn3C)cn1)CC2] | ¹H NMR (400 MHz, DMSO-d₆): δ 8.65 (s, 1H), 8.63 (s, 1H), 8.48 (s, 1H), 7.92 (s, 1H), 4.54 (s, 2H), 4.09 (t, J = 5.6 Hz, 2H), 3.95 (s, 3H), 3.92 (s, 3H), 3.76 (s, 3H), 2.91 (t, J = 5.6 Hz, 2H), 2.21-2.15 (m, 1H), 1.08-1.02 (m, 2H), 0.98-0.93 (m, 2H). LC-MS: m/z 512.2 (M + H)⁺ |
| 53. | I-38 | I-93 | 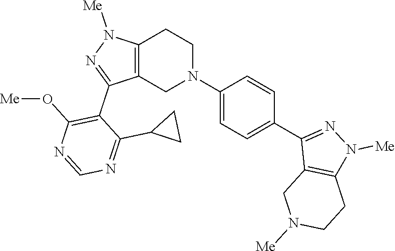 [SMILES: COc1ncnc(C2CC2)c1-c1nn(C)c2c1CN(c1ccc(-c3nn(C)c4c3CN(C)CC4)cc1)CC2] | ¹H NMR (400 MHz, DMSO-d₆): δ 8.62 (s, 1H), 7.39 (d, J = 8.8 Hz, 2H), 7.00 (d, J = 8.8 Hz, 2H), 4.05 (s, 2H), 3.90 (s, 3H), 3.74 (s, 3H), 3.70-3.65 (m, 5H), 3.45 (s, 2H), 2.93 (t, J = 5.2 Hz, 2H), 2.70-2.61 (m, 4H), 2.38 (s, 3H), 2.18-2.12 (m, 1H), 1.05-1.02 (m, 2H), 0.95-0.89 (m, 2H); LC-MS: m/z 511.2 (M + H)⁺ |

The intermediates in below Table 11 were synthesized according to procedure described for compound 1 using appropriate starting material as given here.

TABLE 11

| Int. No. | SM-1 | SM-2 | Structure | Analytical data |
|---|---|---|---|---|
| I-102 | I-46 | I-66 | 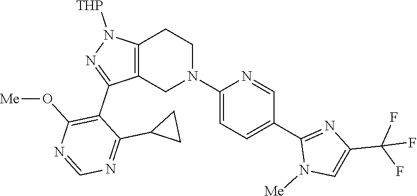 | LC-MS: m/z 581.2 (M + H)+ |
| I-103 | I-38 | I-64 | 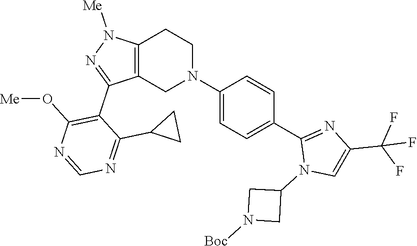 [SMILES: COc1ncnc(C2CC2)c1-c1nn(C)c2c1CN(c1ccc(-c3nc(C(F)(F)F)cn3C3CN(C(=O)OC(C)(C)C)C3)cc1)CC2] | 1H NMR (400 MHz, DMSO-d6): δ 8.62 (s, 1H), 8.35 (s, 1H), 7.34 (d, J = 8.4 Hz, 2H), 7.08 (d, J = 8.8 Hz, 2H), 5.18-5.12 (m, 1H), 4.28-4.22 (m, 2H), 4.14 (s, 2H), 4.12-4.08 (m, 2H), 3.91 (s, 3H), 3.77 (t, J = 5.6 Hz, 2H), 3.75 (s, 3H), 2.86 (t, J = 5.6 Hz, 2H), 2.18-2.12 (m, 1H), 1.41 (s, 9H), 1.06-1.02 (m, 2H), 0.94-0.89 (m, 2H). LC-MS: m/z 651.2 (M + H)+ |
| I-104 | I-38 | I-92 | 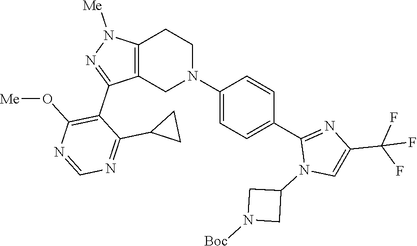 [SMILES: COc1ncnc(C2CC2)c1-c1nn(C)c2c1CN(c1ccc(-c3nc(C(F)(F)F)cn3C3CN(C(=O)OC(C)(C)C)C3)cc1)CC2] | LC-MS: m/z 597.4 (M + H)+ |

### Example 2: Synthesis of 3-(4-cyclopropyl-6-methoxypyrimidin-5-yl)-5-(5-(1-methyl-4-(trifluoromethyl)-1H-imidazol-2-yl)pyridin-2-yl)-4,5,6,7-tetrahydro-1H-pyrazolo[4,3-c]pyridine (Compound-54)

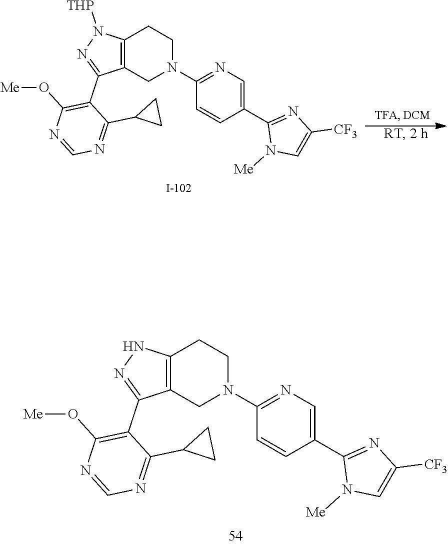

To a stirred solution of intermediate I-102 (0.06 g, 0.103 mmol, 1.0 equiv.) in dichloromethane (3 mL) was added trifluoroacetic acid (0.035 g, 0.30 mmol, 3.0 equiv.) at room temperature. Stirring was continued for 2 hours. The reaction mixture was quenched with sat. sodium bicarbonate solution and extracted with 10% methanol in dichloromethane (3×). The combined organic layers were washed with brine and dried over anhydrous sodium sulphate. The filtered organic layer was concentrated to give crude product. The crude material was washed with diethyl ether and hexane to give the compound 54 (0.015 g) as an off-white solid.

¹H NMR (400 MHz, DMSO-d₆): δ 12.77 (bs, 1H), 8.65 (s, 1H), 8.41 (d, J=2.0 Hz, 1H), 7.88 (s, 1H), 7.85 (dd, J=8.8, 2.8 Hz, 1H), 7.01 (d, J=8.8 Hz, 1H), 4.52 (s, 2H), 4.03 (t, J=5.8 Hz, 2H), 3.92 (s, 3H), 3.75 (s, 3H), 2.85 (t, J=5.6 Hz, 2H), 2.08-2.01 (m, 1H), 1.07-1.02 (m, 2H), 0.96-0.90 (m, 2H). LC-MS: m/z 497.3 (M+H)⁺

### Example 3: Synthesis of 5-(4-(1-(azetidin-3-yl)-4-(trifluoromethyl)-1H-imidazol-2-yl)phenyl)-3-(4-cyclopropyl-6-methoxypyrimidin-5-yl)-1-methyl-4,5,6,7-tetrahydro-1H-pyrazolo[4,3-c]pyridine (Compound-55)

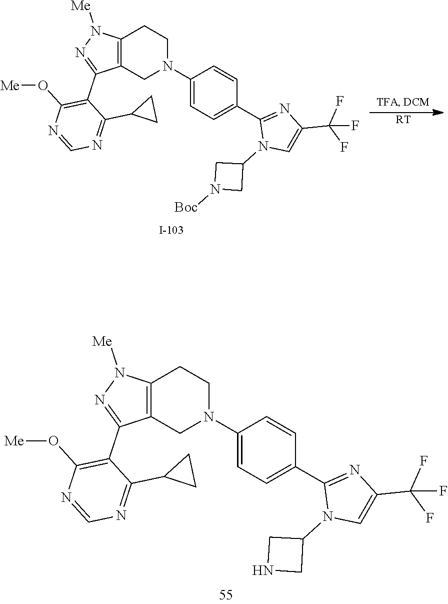

Compound 55 was synthesized from I-103 according to the procedure described for I-36. ¹H NMR (400 MHz, DMSO-d₆): δ 8.63 (s, 1H), 8.01 (s, 1H), 7.35 (d, J=8.4 Hz, 2H), 7.08 (d, J=8.8 Hz, 2H), 4.92-4.85 (m, 1H), 4.15 (s, 2H), 3.92 (s, 3H), 3.77 (t, J=6.0 Hz, 2H), 3.76 (s, 3H), 3.75-3.60 (m, 4H), 2.68 (t, J=5.6 Hz, 2H), 2.18-2.12 (m, 1H), 1.06-1.02 (m, 2H), 0.94-0.89 (m, 2H). LC-MS: m/z 551.2 (M+H)⁺

### Example 4: Synthesis of 3-(4-cyclopropyl-6-methoxypyrimidin-5-yl)-1-methyl-5-(4-(1-methyl-4,5,6,7-tetrahydro-1H-pyrazolo[4,3-c]pyridin-3-yl)phenyl)-4,5,6,7-tetrahydro-1H-pyrazolo[4,3-c]pyridine (Compound-56)

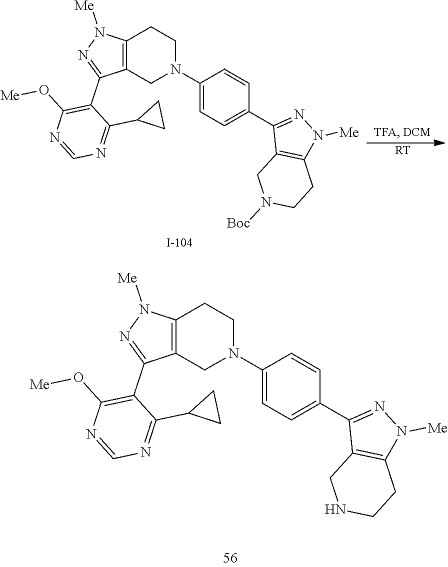 [SMILES: COc1ncnc(C2CC2)c1-c1nn(C)c2c1CN(c1ccc(-c3nn(C)c4c3CN(C(=O)OC(C)(C)C)CC4)cc1)CC2 ; "RT" ; COc1ncnc(C2CC2)c1-c1nn(C)c2c1CN(c1ccc(-c3nn(C)c4c3CNCC4)cc1)CC2]

Compound 56 was synthesized from I-103 according to the procedure described for I-36.

¹H NMR (400 MHz, DMSO-d₆): δ 8.62 (s, 1H), 7.41 (d, J=8.8 Hz, 2H), 7.00 (d, J=8.8 Hz, 2H), 4.05 (s, 2H), 3.91 (s, 3H), 3.75 (s, 2H), 3.70 (s, 3H), 3.68 (t, J=5.6 Hz, 2H), 3.67 (s, 3H), 2.93 (t, J=5.6 Hz, 2H), 2.83 (t, J=5.6 Hz, 2H), 2.55 (t, J=5.2 Hz, 2H), 2.18-2.12 (m, 1H), 1.05-1.02 (m, 2H), 0.95-0.89 (m, 2H). LC-MS: m/z 497.4 (M+H)⁺

### Example 5: 3-(4-cyclopropyl-6-methoxypyrimidin-5-yl)-5-(3-ethyl-4-(1-methyl-4-(trifluoromethyl)-1H-imidazol-2-yl)phenyl)-1-methyl-4,5,6,7-tetrahydro-1H-pyrazolo[4,3-c]pyridine (Compound-57)

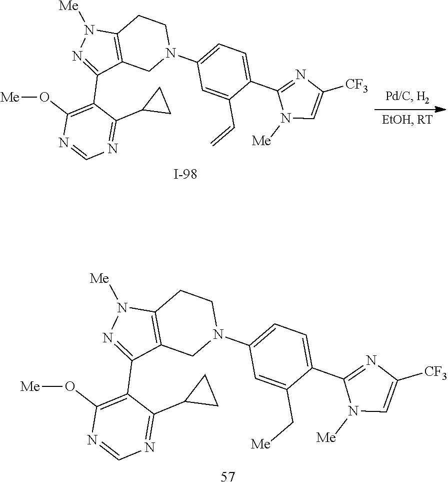

A mixture of intermediate I-98 (0.026 g, 0.049 mmol, 1.0 equiv.) and 10% palladium on charcoal (0.026 g, 0.240 mmol, 5.0 equiv.) in ethanol (5 mL) was stirred under an atmosphere of hydrogen for 6 hours at room temperature. The reaction mixture was filtered through a pad of Celite®. The filtrate was concentrated under reduced pressure to provide crude product, which was purified by prep-TLC using ethyl acetate and hexane as a mobile phase to give pure product ethylbenzene 57 (0.007 g) as an off-white solid.

¹H NMR (400 MHz, DMSO-d₆): δ 8.62 (s, 1H), 7.86 (d, J=1.2 Hz, 1H), 7.14 (d, J=8.8 Hz, 1H), 6.95 (d, J=2.4 Hz, 1H), 6.89 (dd, J=8.8, 2.0 Hz, 1H), 4.13 (s, 2H), 3.91 (s, 3H), 3.80-3.76 (m, 5H), 3.46 (s, 3H), 2.86 (t, J=5.6 Hz, 2H), 2.45 (q, J=7.2 Hz, 2H), 2.18-2.12 (m, 1H), 1.06-1.02 (m, 2H), 0.94 (t, J=7.2 Hz, 3H), 0.94-0.90 (m, 2H). LC-MS: m/z 538.2 (M+H)⁺

### Example 6: Synthesis of 3-(4-cyclopropyl-6-ethoxypyrimidin-5-yl)-1-methyl-5-(4-(1-methyl-4-(trifluoromethyl)-1H-imidazol-2-yl)phenyl)-4,5,6,7-tetrahydro-1H-pyrazolo[4,3-c]pyridine (Compound-58)

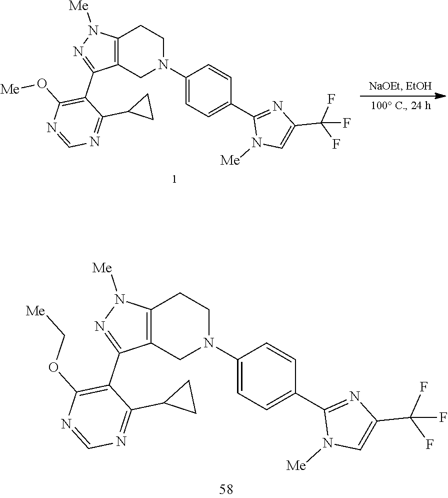 [SMILES: COc1ncnc(C2CC2)c1-c1nn(C)c2c1CN(c1ccc(-c3nc(C(F)(F)F)cn3C)cc1)CC2 ; CCOc1ncnc(C2CC2)c1-c1nn(C)c2c1CN(c1ccc(-c3nc(C(F)(F)F)cn3C)cc1)CC2]

To a stirred solution of compound 1 (0.02 g, 0.039 mmol, 1.0 equiv.) in ethanol (1 mL) was added sodium ethoxide (0.026 g, 0.039 mmol, 10.0 equiv.) at room temperature. The reaction mixture was heated to 100° C. for 24 hours. The reaction mixture was cooled to room temperature, quenched with ice-cold water, and extracted with ethyl acetate (3×). The combined organic layers were dried over anhydrous sodium sulphate and then concentrated to give crude product, which was purified by prep-TLC using ethyl acetate and hexane as a mobile phase to give pure compound 58 (0.005 g) as an off-white solid.

¹H NMR (400 MHz, DMSO-d₆): δ 8.60 (s, 1H), 7.84 (d, J=1.2 Hz, 1H), 7.52 (d, J=8.8 Hz, 2H), 7.08 (d, J=9.2 Hz, 2H), 4.43 (q, J=6.8 Hz, 2H), 4.15 (s, 2H), 3.78 (s, 3H), 3.76 (t, J=5.6 Hz, 2H), 3.74 (s, 3H), 2.86 (t, J=5.6 Hz, 2H), 2.18-2.12 (m, 1H), 1.27 (t, J=6.8 Hz, 3H), 1.06-1.02 (m, 2H), 0.95-0.90 (m, 2H). LC-MS: m/z 524.3 (M+H)⁺

### Example 7: Synthesis of 3-(4-cyclopropyl-6-methoxypyrimidin-5-yl)-1-methyl-5-(5-(1-methyl-4-(trifluoromethyl)-1H-imidazol-2-yl)pyrimidin-2-yl)-4,5,6,7-tetrahydro-1H-pyrazolo[4,3-c]pyridine (Compound-59)

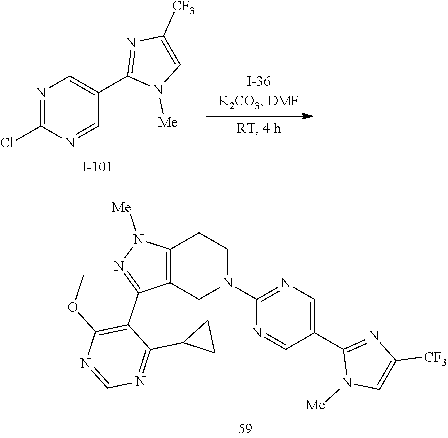 [SMILES: Cn1cc(C(F)(F)F)nc1-c1cnc(Cl)nc1 ; COc1ncnc(C2CC2)c1-c1nn(C)c2c1CN(c1ncc(-c3nc(C(F)(F)F)cn3C)cn1)CC2]

A mixture of intermediate I-101 (0.035 g, 0.133 mmol, 1.0 equiv.), I-36 (0.057 g, 0.20 mmol, 1.5 equiv.) and potassium carbonate (0.055 g, 0.39 mmol, 3.0 equiv.) in DMF (1 mL) was stirred at room temperature for 4 hours. The reaction was quenched with water and then extracted with ethyl acetate (3×). The combined organic layers were dried over anhydrous sodium sulphate and then concentrated to give crude product, which was purified by prep-TLC using ethyl acetate and hexane as a mobile phase to give pure compound 59 (0.03 g) as an off-white solid.

¹H NMR (400 MHz, DMSO-d₆): δ 8.66 (s, 2H), 8.59 (s, 1H), 7.89 (s, 1H), 4.62 (s, 2H), 4.17 (t, J=5.6 Hz, 2H), 3.88 (s, 3H), 3.73 (s, 3H), 3.72 (s, 3H), 2.84 (t, J=5.6 Hz, 2H), 2.18-2.12 (m, 1H), 1.03-0.98 (m, 2H), 0.92-0.89 (m, 2H). LC-MS: m/z 512.2 (M+H)⁺

### Example 8: Synthesis of 3-(4-cyclopropyl-2-methoxypyridin-3-yl)-1-methyl-5-(4-(1-methyl-4-(trifluoromethyl)-1H-imidazol-2-yl)phenyl)-4,5,6,7-tetrahydro-1H-pyrazolo[4,3-c]pyridine (Compound-60)

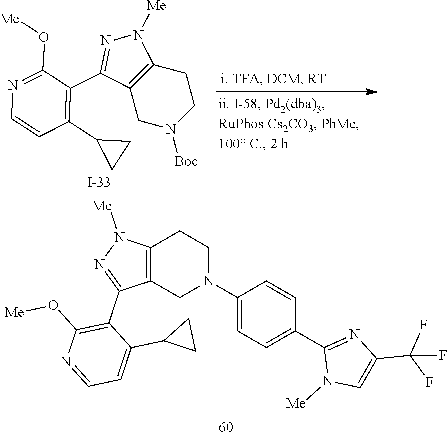

The intermediate I-33 was subjected to procedure described for I-38 to give a free secondary amine, which was coupled with intermediate I-58 according to the procedure described for synthesis of compound 1 to give the compound 60.

¹H NMR (400 MHz, DMSO-d₆): δ 8.04 (d, J=5.2 Hz, 1H), 7.84 (d, J=1.2 Hz, 1H), 7.53 (d, J=8.8 Hz, 2H), 7.06 (d, J=8.8 Hz, 2H), 6.47 (d, J=5.6 Hz, 1H), 4.10 (s, 2H), 3.82 (s, 3H), 3.77 (t, J=5.6 Hz, 2H), 3.74 (s, 3H), 3.32 (s, 3H), 2.84 (t, J=5.6 Hz, 2H), 1.98-1.89 (m, 1H), 0.95-0.90 (m, 2H), 0.89-0.84 (m, 2H). LC-MS: m/z 509.3 (M+H)⁺

The compounds in Table 12 were prepared according to the procedure described in compound 60, using the appropriate starting material as given here.

TABLE 12

| Compound | ISM-1 | SM-2 | Structure | Analytical data |
|---|---|---|---|---|
| 61. | I-35 | I-58 | 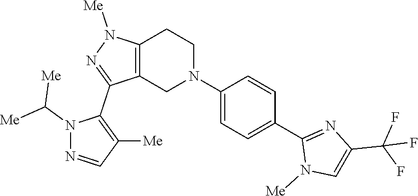 [SMILES: Cc1cnn(C(C)C)c1-c1nn(C)c2c1CN(c1ccc(-c3nc(C(F)(F)F)cn3C)cc1)CC2] | 1H NMR (400 MHz, DMSO-d6): δ 7.83 (s, 1H), 7.51 (d, J = 8.8 Hz, 2H), 7.37 (s, 1H), 7.05 (d, J = 9.2 Hz, 2H), 4.49-4.45 (m, 1H), 4.15 (s, 2H), 3.78 (t, J = 5.6 Hz, 2H), 3.77 (s, 3H), 3.73 (s, 3H), 2.88 (t, J = 5.6 Hz, 2H), 1.94 (s, 3H), 1.31 (d, J = 6.8 Hz, 6H). LC-MS: m/z 484.3 (M + H)+ |
| 62. | I-35 | I-65 | 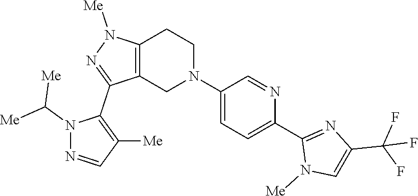 [SMILES: Cc1cnn(C(C)C)c1-c1nn(C)c2c1CN(c1ccc(-c3nc(C(F)(F)F)cn3C)nc1)CC2] | 1H NMR (400 MHz, DMSO-d6): δ 8.35 (d, J = 2.4 Hz, 1H), 7.86 (s, 1H), 7.81 (d, J = 9.6 Hz, 1H), 7.44 (dd, J = 9.2, 3.2 Hz, 1H), 7.34 (s, 1H), 4.48-4.40 (m, 1H), 4.17 (s, 2H), 3.98 (s, 3H), 3.81 (t, J = 5.6 Hz, 2H), 3.74 (s, 3H), 2.87 (t, J = 5.6 Hz, 2H), 1.91 (s, 3H), 1.27 (d, J = 6.8 Hz, 6H). LC-MS: m/z 485.4 (M + H)+ |

### Biology

Biochemical Assay: The compounds were evaluated for their potential to inhibit USP1-UAF1 complex (Boston Biochem, Catalog: E568-050) using a fluorescence-based assay. The final concentrations of USP1-UAF1 complex protein and substrate Ubiquitin-Rhodamine-110 (R&D systems, Catalog U-555-050) used in the assay were 0.45 and 150 nM respectively. 50 mM HEPES pH. 7.5, 100 mM NaCl, 0.5 mM EDTA, 1 mM TCEP, 10% BSA, 0.01% Tween 20 buffer was used in the assay. The total assay volume was 20 μL. The Compounds were initially prepared in 100% DMSO and appropriate dilution were made by ⅓^{rd} serial dilutions from the stock to determine the IC₅₀ value. The final DMSO concentration in the assay was 1%. The compounds were pre-incubated with USP1-UAF1 complex at 25° C. for 15 min. After preincubation, required concentration of substrate was added and incubated at 25° C. for 60 min. Fluorescence at Excitation: 485 nm, Emission: 535 nm was measured in Victor-5 from Perkin Elmer.

To determine IC₅₀ values, dose response curves were generated by plotting percentage inhibition as a function of inhibitor concentration and the data was fitted to sigmoidal non-linear regression equation (variable slope) using Graph Pad prism software V8.

The compounds were screened by the above-mentioned assay procedure. The % inhibition at 1.2 μM concentration and the IC₅₀ values of the compounds are summarized in the Table 20 below wherein “A” refers to an IC₅₀ value less than 0.05 μM, “B” refers to IC₅₀ value in range of 0.05 μM to 0.1 μM and “C” refers to an IC₅₀ value greater than 0.1 μM. The results are given below in Table 13.

TABLE 13
% inhibition and IC₅₀ values

| Compound No. | % Inhibition @ 1.2 μM | USP1-UAF1 IC₅₀ (μM) |
|---|---|---|
| 1 | 98 | B |
| 2 | 100 @ 1 μM | A |
| 3 | 99 | A |
| 4 | 81 | C |
| 5 | 100 | A |
| 6 | 100 | A |
| 7 | 100 | A |
| 8 | 100 | A |
| 9 | 99 | A |
| 10 | 88 | C |
| 11 | 86 | C |
| 12 | 98 | A |
| 13 | 98 | A |
| 14 | 99 | B |
| 15 | 96 | B |
| 16 | 99 | A |
| 17 | 100 | A |
| 18 | 99 | A |
| 19 | 99 | A |
| 20 | 99 | A |
| 21 | 97 | A |
| 22 | 39 | C |
| 23 | 3 | C |
| 24 | 98 | A |
| 25 | 92 | C |
| 26 | 90 | C |
| 27 | 98 | A |
| 28 | 90 | C |
| 29 | 90 | C |
| 30 | 98 | A |
| 31 | 86 | C |
| 32 | 89 | C |
| 33 | 90 | B |
| 34 | 75 | C |
| 35 | 76 | C |
| 36 | 99 | A |
| 37 | 98 | B |
| 38 | 99 | A |
| 39 | 98 | B |
| 40 | 97 | B |
| 41 | 100 | A |
| 42 | 96 | B |
| 43 | 98 | B |
| 44 | 100 | A |
| 45 | 98 | A |
| 46 | 96 | B |
| 47 | 95 | B |
| 48 | 97 | B |
| 49 | 14 | C |
| 50 | 20 | C |
| 51 | 95 | B |
| 52 | 96 | B |
| 53 | 42 | C |
| 54 | 91 | C |
| 55 | 87 | C |
| 56 | 5 | C |
| 57 | 87 | C |
| 58 | 98 | A |
| 59 | 95 | B |
| 60 | 92 | A |
| 61 | 98 | A |
| 62 | 95 | B |

### INCORPORATION BY REFERENCE

All publications and patents mentioned herein are hereby incorporated by reference in their entirety as if each individual publication or patent were specifically and individually indicated to be incorporated by reference. In case of conflict, the present disclosure, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A compound of formula (VI): 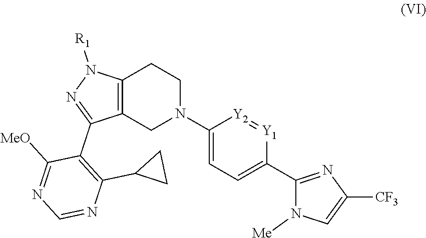
or a pharmaceutically acceptable salt thereof or a stereoisomer thereof, wherein:
R₁ is selected from the group consisting of C₂-C₄ alkyl, C₃-C₅ cycloalkyl, and arylalkyl; and
Y₁ and Y₂ are CH or N, wherein at least one of Y₁ and Y₂ is CH;
with the proviso that the compound of formula V is not 3-(4-cyclopropyl-6-methoxypyrimidin-5-yl)-1-ethyl-5-(5-(1-methyl-4-(trifluoromethyl)-1H-imidazol-2-yl)pyridin-2-yl)-4,5,6,7-tetrahydro-1H-pyrazolo[4,3-c]pyridine.

2. The compound of claim 1, selected from the group consisting of: 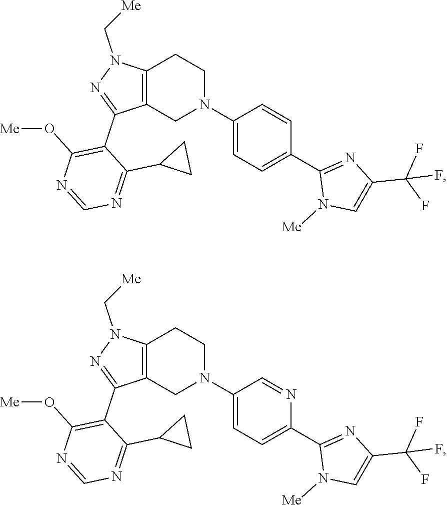 [SMILES: CCn1nc(-c2c(OC)ncnc2C2CC2)c2c1CCN(c1ccc(-c3nc(C(F)(F)F)cn3C)cc1)C2 ; CCn1nc(-c2c(OC)ncnc2C2CC2)c2c1CCN(c1ccc(-c3nc(C(F)(F)F)cn3C)nc1)C2] 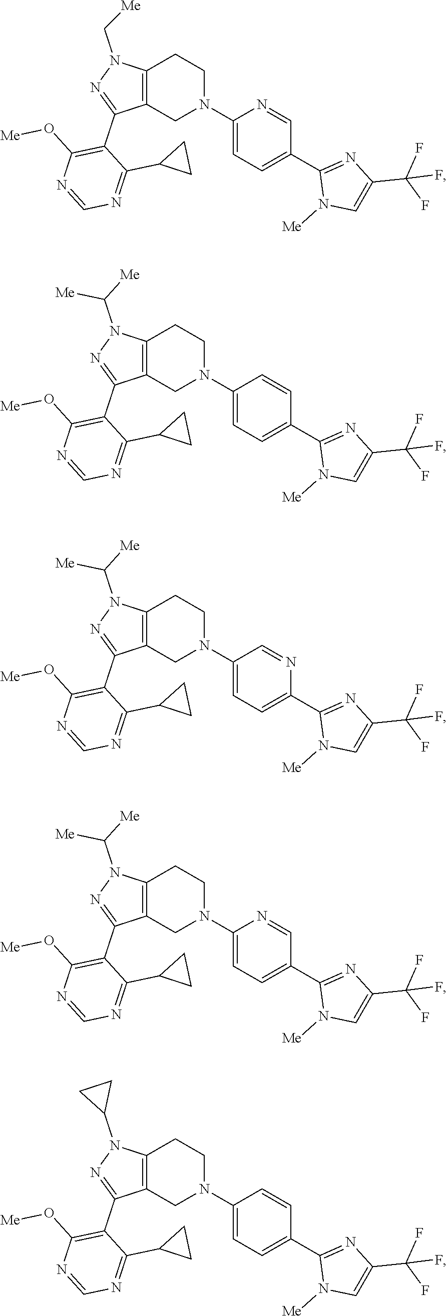 [SMILES: CCn1nc(-c2c(OC)ncnc2C2CC2)c2c1CCN(c1ccc(-c3nc(C(F)(F)F)cn3C)cn1)C2 ; COc1ncnc(C2CC2)c1-c1nn(C(C)C)c2c1CN(c1ccc(-c3nc(C(F)(F)F)cn3C)cc1)CC2 ; COc1ncnc(C2CC2)c1-c1nn(C(C)C)c2c1CN(c1ccc(-c3nc(C(F)(F)F)cn3C)nc1)CC2 ; COc1ncnc(C2CC2)c1-c1nn(C(C)C)c2c1CN(c1ccc(-c3nc(C(F)(F)F)cn3C)cn1)CC2 ; COc1ncnc(C2CC2)c1-c1nn(C2CC2)c2c1CN(c1ccc(-c3nc(C(F)(F)F)cn3C)cc1)CC2] 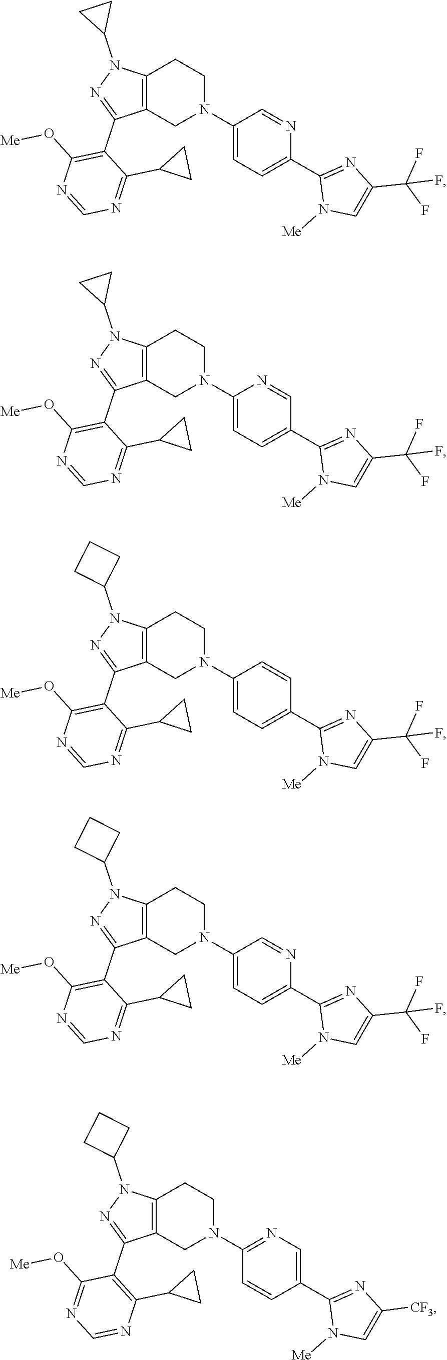 [SMILES: COc1ncnc(C2CC2)c1-c1nn(C2CC2)c2c1CN(c1ccc(-c3nc(C(F)(F)F)cn3C)nc1)CC2 ; COc1ncnc(C2CC2)c1-c1nn(C2CC2)c2c1CN(c1ccc(-c3nc(C(F)(F)F)cn3C)cn1)CC2 ; COc1ncnc(C2CC2)c1-c1nn(C2CCC2)c2c1CN(c1ccc(-c3nc(C(F)(F)F)cn3C)cc1)CC2 ; COc1ncnc(C2CC2)c1-c1nn(C2CCC2)c2c1CN(c1ccc(-c3nc(C(F)(F)F)cn3C)nc1)CC2 ; COc1ncnc(C2CC2)c1-c1nn(C2CCC2)c2c1CN(c1ccc(-c3nc(C(F)(F)F)cn3C)cn1)CC2] 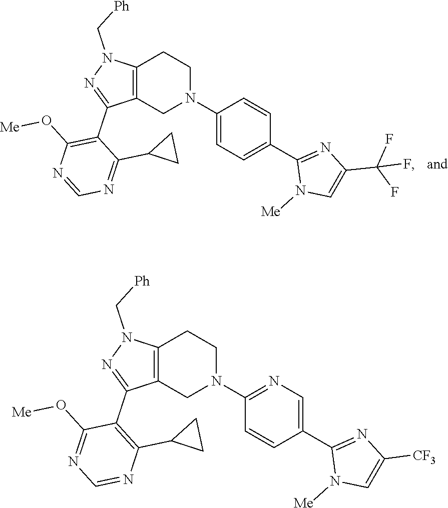 [SMILES: COc1ncnc(C2CC2)c1-c1nn(Cc2ccccc2)c2c1CN(c1ccc(-c3nc(C(F)(F)F)cn3C)cc1)CC2 ; COc1ncnc(C2CC2)c1-c1nn(Cc2ccccc2)c2c1CN(c1ccc(-c3nc(C(F)(F)F)cn3C)cn1)CC2]
or a pharmaceutically acceptable salt thereof or a stereoisomer thereof.

3. A compound of formula (III): 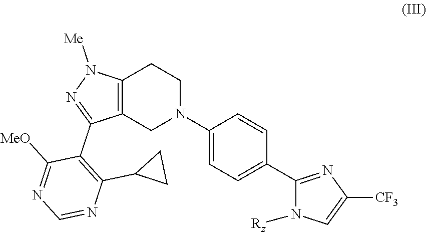
or a pharmaceutically acceptable salt thereof or a stereoisomer thereof, wherein:
R_{z} is selected from the group consisting of ethyl, isopropyl, C₃-C₅ cycloalkyl, alkoxyalkyl, dialkylaminoalkyl, and C₃-C₅ heterocycloalkyl optionally substituted with methyl.

4. The compound of claim 3, selected from the group consisting of: 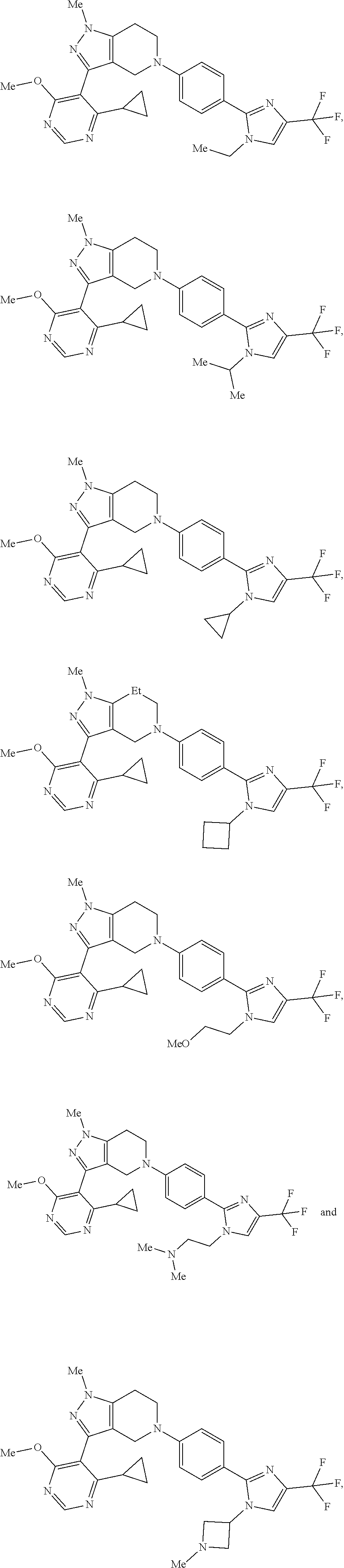
or a pharmaceutically acceptable salt thereof or a stereoisomer thereof.

5. A compound of formula (IV): 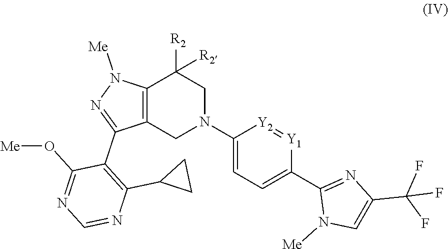
or a pharmaceutically acceptable salt thereof or a stereoisomer thereof, wherein:
R₂ and R_{2′} are independently selected from H, methyl, and ethyl, and at least one of R₂ and R_{2′} is methyl or ethyl; and
Y₁ and Y₂ are CH or N, wherein at least one of Y₁ and Y₂ is CH.

6. The compound of claim 5, selected from the group consisting of: 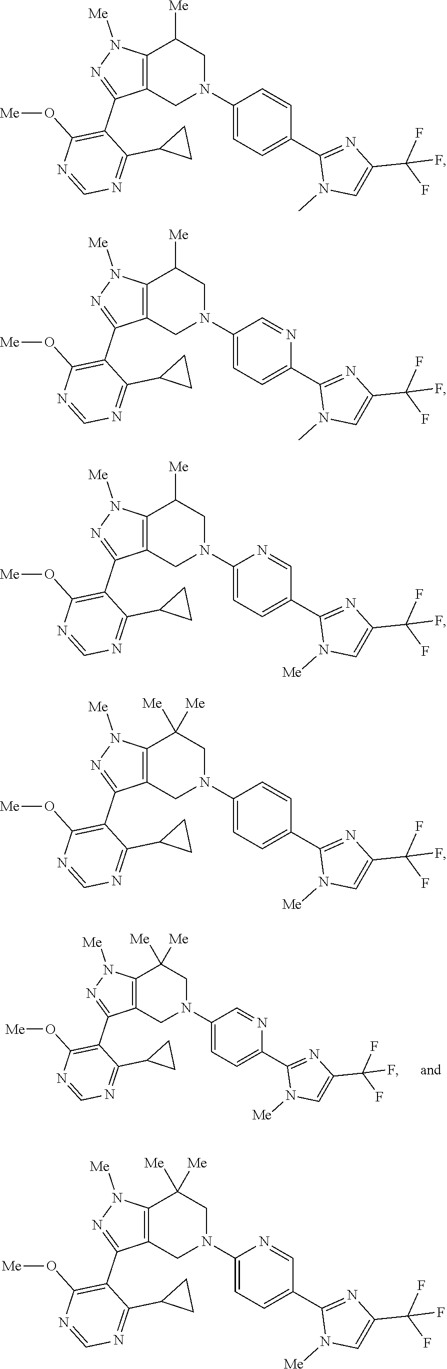 [SMILES: COc1ncnc(C2CC2)c1-c1nn(C)c2c1CN(c1ccc(-c3nc(C(F)(F)F)cn3C)cc1)CC2C ; COc1ncnc(C2CC2)c1-c1nn(C)c2c1CN(c1ccc(-c3nc(C(F)(F)F)cn3C)nc1)CC2C ; COc1ncnc(C2CC2)c1-c1nn(C)c2c1CN(c1ccc(-c3nc(C(F)(F)F)cn3C)cn1)CC2C ; COc1ncnc(C2CC2)c1-c1nn(C)c2c1CN(c1ccc(-c3nc(C(F)(F)F)cn3C)cc1)CC2(C)C ; COc1ncnc(C2CC2)c1-c1nn(C)c2c1CN(c1ccc(-c3nc(C(F)(F)F)cn3C)nc1)CC2(C)C ; COc1ncnc(C2CC2)c1-c1nn(C)c2c1CN(c1ccc(-c3nc(C(F)(F)F)cn3C)cn1)CC2(C)C]
or a pharmaceutically acceptable salt thereof or a stereoisomer thereof.

7. A compound of formula (V): 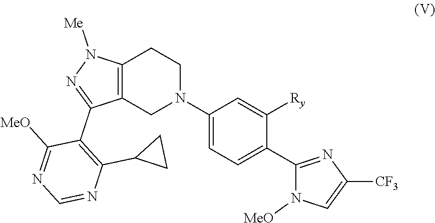
or a pharmaceutically acceptable salt thereof or a stereoisomer thereof, wherein:
R_{y} is selected from the group consisting of Cl, Br, alkoxy, and dialkylamino.

8. The compound of claim 7, selected from the group consisting of: 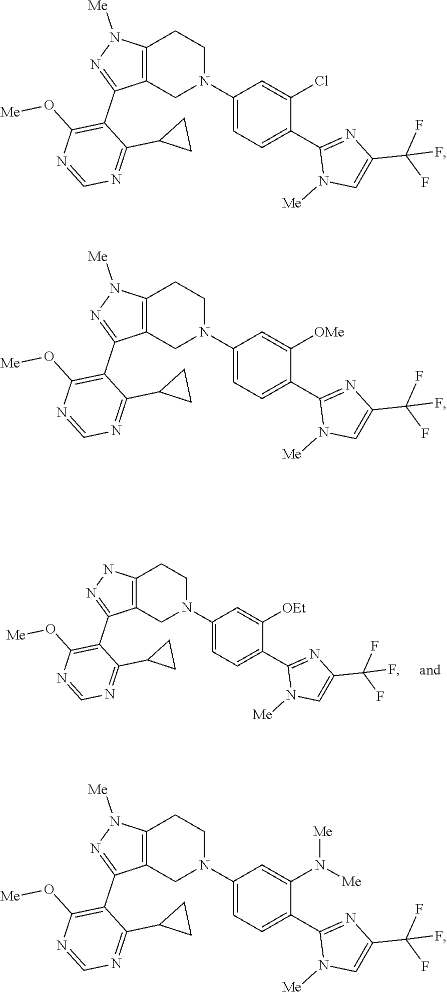
or a pharmaceutically acceptable salt thereof or a stereoisomer thereof.

9. A compound of formula (II): 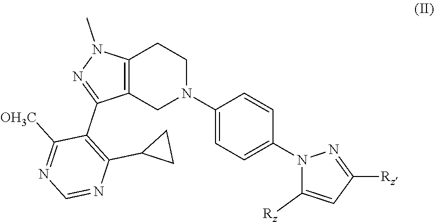
or a pharmaceutically acceptable salt thereof or a stereoisomer thereof, wherein:
R_{z} is selected from the group consisting of alkyl, alkoxy and dialkylamino; and
R_{z′} is CH₃ or CF₃.

10. The compound of claim 9, selected from the group consisting of: 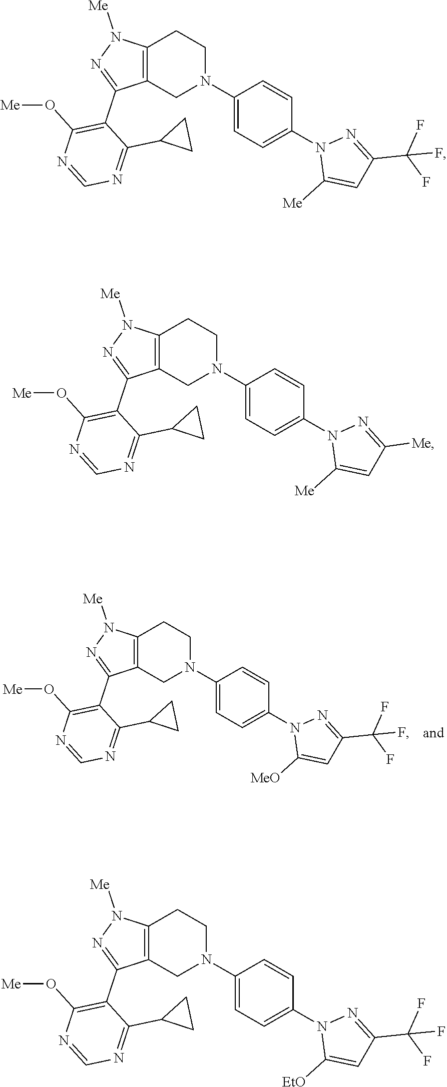 [SMILES: COc1ncnc(C2CC2)c1-c1nn(C)c2c1CN(c1ccc(-n3nc(C(F)(F)F)cc3C)cc1)CC2 ; COc1ncnc(C2CC2)c1-c1nn(C)c2c1CN(c1ccc(-n3nc(C)cc3C)cc1)CC2 ; COc1ncnc(C2CC2)c1-c1nn(C)c2c1CN(c1ccc(-n3nc(C(F)(F)F)cc3OC)cc1)CC2 ; "and" ; CCOc1cc(C(F)(F)F)nn1-c1ccc(N2CCc3c(c(-c4c(OC)ncnc4C4CC4)nn3C)C2)cc1]
or a pharmaceutically acceptable salt thereof or a stereoisomer thereof.

11. A compound selected from the group consisting of

| No. | Compound |
|---|---|
| 2. | 3-(2-cyclopropyl-6-methoxyphenyl)-1-methyl-5-(4-(1-methyl-4-(trifluoromethyl)-1H- |
| | imidazol-2-yl)phenyl)-4,5,6,7-tetrahydro-1H-pyrazolo[4,3-c]pyridine |
| 4. | 3-(2-isopropylpyridin-3-yl)-1-methyl-5-(6-(1-methyl-4-(trifluoromethyl)-1H-imidazol- |
| | 2-yl)pyridin-3-yl)-4,5,6,7-tetrahydro-1H-pyrazolo[4,3-c]pyridine |
| 5. | 3-(4-cyclopropyl-6-methoxypyrimidin-5-yl)-5-(4-(1-ethyl-4-(trifluoromethyl)-1H- |
| | imidazol-2-yl)phenyl)-1-methyl-4,5,6,7-tetrahydro-1H-pyrazolo[4,3-c]pyridine |
| 6. | 3-(4-cyclopropyl-6-methoxypyrimidin-5-yl)-5-(4-(1-isopropyl-4-(trifluoromethyl)-1H- |
| | imidazol-2-yl)phenyl)-1-methyl-4,5,6,7-tetrahydro-1H-pyrazolo[4,3-c]pyridine |
| 7. | 5-(4-(1-cyclopropyl-4-(trifluoromethyl)-1H-imidazol-2-yl)phenyl)-3-(4-cyclopropyl-6- |
| | methoxypyrimidin-5-yl)-1-methyl-4,5,6,7-tetrahydro-1H-pyrazolo[4,3-c]pyridine |
| 8. | 5-(4-(1-cyclobutyl-4-(trifluoromethyl)-1H-imidazol-2-yl)phenyl)-3-(4-cyclopropyl-6- |
| | methoxypyrimidin-5-yl)-1-methyl-4,5,6,7-tetrahydro-1H-pyrazolo[4,3-c]pyridine |
| 9. | 3-(4-cyclopropyl-6-methoxypyrimidin-5-yl)-5-(4-(1-(2-methoxyethyl)-4- |
| | (trifluoromethyl)-1H-imidazol-2-yl)phenyl)-1-methyl-4,5,6,7-tetrahydro-1H- |
| | pyrazolo[4,3-c]pyridine |
| 10. | 2-(2-(4-(3-(4-cyclopropyl-6-methoxypyrimidin-5-yl)-1-methyl-1,4,6,7-tetrahydro-5H- |
| | pyrazolo[4,3-c]pyridin-5-yl)phenyl)-4-(trifluoromethyl)-1H-imidazol-1-yl)-N,N- |
| | dimethylethan-1-amine |
| 11. | 3-(4-cyclopropyl-6-methoxypyrimidin-5-yl)-1-methyl-5-(4-(1-(1-methylazetidin-3-yl)- |
| | 4-(trifluoromethyl)-1H-imidazol-2-yl)phenyl)-4,5,6,7-tetrahydro-1H-pyrazolo[4,3- |
| | c]pyridine |
| 16. | 5-(5-(1-cyclobutyl-4-(trifluoromethyl)-1H-imidazol-2-yl)pyridin-2-yl)-3-(4- |
| | cyclopropyl-6-methoxypyrimidin-5-yl)-1-methyl-4,5,6,7-tetrahydro-1H-pyrazolo[4,3- |
| | clpyridine |
| 17. | 3-(4-cyclopropyl-6-methoxypyrimidin-5-yl)-1-methyl-5-(4-(5-methyl-3- |
| | (trifluoromethyl)-1H-pyrazol-1-yl)phenyl)-4,5,6,7-tetrahydro-1H-pyrazolo[4,3- |
| | c]pyridine |
| 18. | 3-(4-cyclopropyl-6-methoxypyrimidin-5-yl)-5-(4-(3,5-dimethyl-1H-pyrazol-1- |
| | yl)phenyl)-1-methyl-4,5,6,7-tetrahydro-1H-pyrazolo[4,3-c]pyridine |
| 19. | 3-(4-cyclopropyl-6-methoxypyrimidin-5-yl)-5-(4-(5-methoxy-3-(trifluoromethyl)-1H- |
| | pyrazol-1-yl)phenyl)-1-methyl-4,5,6,7-tetrahydro-1H-pyrazolo[4,3-c]pyridine |
| 20. | 3-(4-cyclopropyl-6-methoxypyrimidin-5-yl)-5-(4-(5-ethoxy-3-(trifluoromethyl)-1H- |
| | pyrazol-1-yl)phenyl)-1-methyl-4,5,6,7-tetrahydro-1H-pyrazolo[4,3-c]pyridine |
| 21. | 5-(4-(4-chloro-1-methyl-1H-imidazol-2-yl)phenyl)-3-(4-cyclopropyl-6- |
| | methoxypyrimidin-5-yl)-1-methyl-4,5,6,7-tetrahydro-1H-pyrazolo[4,3-c]pyridine |
| 22. | 3-(4-cyclopropyl-6-methoxypyrimidin-5-yl)-5-(2′-methoxy-[3,3′-bipyridin]-6-yl)-1- |
| | methyl-4,5,6,7-tetrahydro-1H-pyrazolo[4,3-c]pyridine |
| 23. | 4-(6-(3-(4-cyclopropyl-6-methoxypyrimidin-5-yl)-1-methyl-1,4,6,7-tetrahydro-5H- |
| | pyrazolo[4,3-c]pyridin-5-yl)pyridin-3-yl)-3,5-dimethylisoxazole |
| 24. | 3-(4-cyclopropyl-6-methoxypyrimidin-5-yl)-1-ethyl-5-(4-(1-methyl-4- |
| | (trifluoromethyl)-1H-imidazol-2-yl)phenyl)-4,5,6,7-tetrahydro-1H-pyrazolo[4,3- |
| | c]pyridine |
| 25. | 3-(4-cyclopropyl-6-methoxypyrimidin-5-yl)-1-ethyl-5-(6-(1-methyl-4- |
| | (trifluoromethyl)-1H-imidazol-2-yl)pyridin-3-yl)-4,5,6,7-tetrahydro-1H-pyrazolo[4,3- |
| | c]pyridine |
| 27. | 3-(4-cyclopropyl-6-methoxypyrimidin-5-yl)-1-isopropyl-5-(4-(1-methyl-4- |
| | (trifluoromethyl)-1H-imidazol-2-yl)phenyl)-4,5,6,7-tetrahydro-1H-pyrazolo[4,3- |
| | c]pyridine |
| 28. | 3-(4-cyclopropyl-6-methoxypyrimidin-5-yl)-1-isopropyl-5-(6-(1-methyl-4- |
| | (trifluoromethyl)-1H-imidazol-2-yl)pyridin-3-yl)-4,5,6,7-tetrahydro-1H-pyrazolo[4,3- |
| | c]pyridine |
| 29. | 3-(4-cyclopropyl-6-methoxypyrimidin-5-yl)-1-isopropyl-5-(5-(1-methyl-4- |
| | (trifluoromethyl)-1H-imidazol-2-yl)pyridin-2-yl)-4,5,6,7-tetrahydro-1H-pyrazolo[4,3- |
| | c]pyridine |
| 30. | 1-cyclopropyl-3-(4-cyclopropyl-6-methoxypyrimidin-5-yl)-5-(4-(1-methyl-4- |
| | (trifluoromethyl)-1H-imidazol-2-yl)phenyl)-4,5,6,7-tetrahydro-1H-pyrazolo[4,3- |
| | c]pyridine |
| 31. | 1-cyclopropyl-3-(4-cyclopropyl-6-methoxypyrimidin-5-yl)-5-(6-(1-methyl-4- |
| | (trifluoromethyl)-1H-imidazol-2-yl)pyridin-3-yl)-4,5,6,7-tetrahydro-1H-pyrazolo[4,3- |
| | c]pyridine |
| 32. | 1-cyclopropyl-3-(4-cyclopropyl-6-methoxypyrimidin-5-yl)-5-(5-(1-methyl-4- |
| | (trifluoromethyl)-1H-imidazol-2-yl)pyridin-2-yl)-4,5,6,7-tetrahydro-1H-pyrazolo[4,3- |
| | c]pyridine |
| 33. | 1-cyclobutyl-3-(4-cyclopropyl-6-methoxypyrimidin-5-yl)-5-(4-(1-methyl-4- |
| | (trifluoromethyl)-1H-imidazol-2-yl)phenyl)-4,5,6,7-tetrahydro-1H-pyrazolo[4,3- |
| | c]pyridine |
| 34. | 1-cyclobutyl-3-(4-cyclopropyl-6-methoxypyrimidin-5-yl)-5-(6-(1-methyl-4- |
| | (trifluoromethyl)-1H-imidazol-2-yl)pyridin-3-yl)-4,5,6,7-tetrahydro-1H-pyrazolo[4,3- |
| | c]pyridine |
| 35. | 1-cyclobutyl-3-(4-cyclopropyl-6-methoxypyrimidin-5-yl)-5-(5-(1-methyl-4- |
| | (trifluoromethyl)-1H-imidazol-2-yl)pyridin-2-yl)-4,5,6,7-tetrahydro-1H-pyrazolo[4,3- |
| | c]pyridine |
| 36. | 1-benzyl-3-(4-cyclopropyl-6-methoxypyrimidin-5-yl)-5-(4-(1-methyl-4-. |
| | (trifluoromethyl)-1H-imidazol-2-yl)phenyl)-4,5,6,7-tetrahydro-1H-pyrazolo[4,3- |
| | c]pyridine |
| 37. | 1-benzyl-3-(4-cyclopropyl-6-methoxypyrimidin-5-yl)-5-(5-(1-methyl-4- |
| | (trifluoromethyl)-1H-imidazol-2-yl)pyridin-2-yl)-4,5,6,7-tetrahydro-1H-pyrazolo[4,3- |
| | c]pyridine |
| 38. | 3-(4-cyclopropyl-6-methoxypyrimidin-5-yl)-1,7-dimethyl-5-(4-(1-methyl-4- |
| | (trifluoromethyl)-1H-imidazol-2-yl)phenyl)-4,5,6,7-tetrahydro-1H-pyrazolo[4,3- |
| | c]pyridine |
| 39. | 3-(4-cyclopropyl-6-methoxypyrimidin-5-yl)-1,7-dimethyl-5-(6-(1-methyl-4- |
| | (trifluoromethyl)-1H-imidazol-2-yl)pyridin-3-yl)-4,5,6,7-tetrahydro-1H-pyrazolo[4,3- |
| | c]pyridine |
| 40. | 3-(4-cyclopropyl-6-methoxypyrimidin-5-yl)-1,7-dimethyl-5-(5-(1-methyl-4- |
| | (trifluoromethyl)-1H-imidazol-2-yl)pyridin-2-yl)-4,5,6,7-tetrahydro-1H-pyrazolo[4,3- |
| | c]pyridine |
| 41. | 3-(4-cyclopropyl-6-methoxypyrimidin-5-yl)-1,7,7-trimethyl-5-(4-(1-methyl-4- |
| | (trifluoromethyl)-1H-imidazol-2-yl)phenyl)-4,5,6,7-tetrahydro-1H-pyrazolo[4,3- |
| | c]pyridine |
| 42. | 3-(4-cyclopropyl-6-methoxypyrimidin-5-yl)-1,7,7-trimethyl-5-(6-(1-methyl-4- |
| | (trifluoromethyl)-1H-imidazol-2-yl)pyridin-3-yl)-4,5,6,7-tetrahydro-1H-pyrazolo[4,3- |
| | c]pyridine |
| 43. | 3-(4-cyclopropyl-6-methoxypyrimidin-5-yl)-1,7,7-trimethyl-5-(5-(1-methyl-4- |
| | (trifluoromethyl)-1H-imidazol-2-yl)pyridin-2-yl)-4,5,6,7-tetrahydro-1H-pyrazolo[4,3- |
| | c]pyridine |
| 45. | 5-(3-chloro-4-(1-methyl-4-(trifluoromethyl)-1H-imidazol-2-yl)phenyl)-3-(4- |
| | cyclopropyl-6-methoxypyrimidin-5-yl)-1-methyl-4,5,6,7-tetrahydro-1H-pyrazolo[4,3- |
| | c]pyridine |
| 46. | 3-(4-cyclopropyl-6-methoxypyrimidin-5-yl)-5-(3-methoxy-4-(1-methyl-4- |
| | (trifluoromethyl)-1H-imidazol-2-yl)phenyl)-1-methyl-4,5,6,7-tetrahydro-1H- |
| | pyrazolo[4,3-c]pyridine |
| 47. | 3-(4-cyclopropyl-6-methoxypyrimidin-5-yl)-5-(3-ethoxy-4-(1-methyl-4- |
| | (trifluoromethyl)-1H-imidazol-2-yl)phenyl)-1-methyl-4,5,6,7-tetrahydro-1H- |
| | pyrazolo[4,3-c]pyridine |
| 48. | 5-(3-(4-cyclopropyl-6-methoxypyrimidin-5-yl)-1-methyl-1,4,6,7-tetrahydro-5H- |
| | pyrazolo[4,3-c]pyridin-5-yl)-N,N-dimethyl-2-(1-methyl-4-(trifluoromethyl)-1H- |
| | imidazol-2-yl)aniline |
| 49. | 3-(4-cyclopropyl-6-methoxypyrimidin-5-yl)-1-methyl-5-(5-(1-methyl-4- |
| | (trifluoromethyl)-1H-imidazol-2-yl)thiophen-3-yl)-4,5,6,7-tetrahydro-1H- |
| | pyrazolo[4,3-c]pyridine |
| 50. | 4-(3-(4-cyclopropyl-6-methoxypyrimidin-5-yl)-1-methyl-1,4,6,7-tetrahydro-5H- |
| | pyrazolo[4,3-c]pyridin-5-yl)-2-(1-methyl-4-(trifluoromethyl)-1H-imidazol-2- |
| | yl)thiazole |
| 51. | 3-(4-cyclopropyl-6-methoxypyrimidin-5-yl)-1-methyl-5-(2-(5-methyl-3- |
| | (trifluoromethyl)-1H-pyrazol-1-yl)pyrimidin-5-yl)-4,5,6,7-tetrahydro-1H- |
| | pyrazolo[4,3-c]pyridine |
| 53. | 3-(4-cyclopropyl-6-methoxypyrimidin-5-yl)-5-(4-(1,5-dimethyl-4,5,6,7-tetrahydro-1H- |
| | pyrazolo[4,3-c]pyridin-3-yl)phenyl)-1-methyl-4,5,6,7-tetrahydro-1H-pyrazolo[4,3- |
| | c]pyridine |
| 54. | 3-(4-cyclopropyl-6-methoxypyrimidin-5-yl)-5-(4-(1-methyl-4-(trifluoromethyl)-1H- |
| | imidazol-2-yl)phenyl)-4,5,6,7-tetrahydro-1H-pyrazolo[4,3-c]pyridine |
| 55. | 5-(4-(1-(azetidin-3-yl)-4-(trifluoromethyl)-1H-imidazol-2-yl)phenyl)-3-(4- |
| | cyclopropyl-6-methoxypyrimidin-5-yl)-1-methyl-4,5,6,7-tetrahydro-1H-pyrazolo[4,3- |
| | c]pyridine |
| 56. | 3-(4-cyclopropyl-6-methoxypyrimidin-5-yl)-1-methyl-5-(4-(1-methyl-4,5,6,7- |
| | tetrahydro-1H-pyrazolo[4,3-c]pyridin-3-yl)phenyl)-4,5,6,7-tetrahydro-1H- |
| | pyrazolo[4,3-c]pyridine |
| 57. | 3-(4-cyclopropyl-6-methoxypyrimidin-5-yl)-5-(3-ethyl-4-(1-methyl-4- |
| | (trifluoromethyl)-1H-imidazol-2-yl)phenyl)-1-methyl-4,5,6,7-tetrahydro-1H- |
| | pyrazolo[4,3-c]pyridine |
| 59. | 3-(4-cyclopropyl-6-methoxypyrimidin-5-yl)-1-methyl-5-(5-(1-methyl-4- |
| | (trifluoromethyl)-1H-imidazol-2-yl)pyrimidin-2-yl)-4,5,6,7-tetrahydro-1H- |
| | pyrazolo[4,3-c]pyridine |
| 60. | 3-(4-cyclopropyl-2-methoxypyridin-3-yl)-1-methyl-5-(4-(1-methyl-4- |
| | (trifluoromethyl)-1H-imidazol-2-yl)phenyl)-4,5,6,7-tetrahydro-1H-pyrazolo[4,3- |
| | c]pyridine |
| 61. | 3-(4-cyclopropyl-2-methoxypyridin-3-yl)-1-methyl-5-(4-(1-methyl-4- |
| | (trifluoromethyl)-1H-imidazol-2-yl)phenyl)-4,5,6,7-tetrahydro-1H-pyrazolo[4,3- |
| | c]pyridine |
| 62. | 3-(1-isopropyl-4-methyl-1H-pyrazol-5-yl)-1-methyl-5-(4-(1-methyl-4- |
| | (trifluoromethyl)-1H-imidazol-2-yl)phenyl)-4,5,6,7-tetrahydro-1H-pyrazolo[4,3- |
| | c]pyridine; and |

12. A compound of formula (VII): 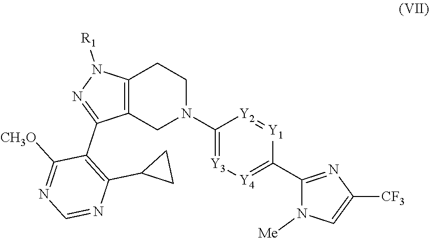
or a pharmaceutically acceptable salt thereof or a stereoisomer thereof, wherein:
R₁ is selected from the group consisting of alkoxyalkyl, dialkylaminoalkyl, cycloalkyl, heterocycloalkyl, arylalkyl substituted by one or more halogen, arylalkyl substituted by one or more alkoxy, and —CH₂-heteroaryl; and
Y₁, Y₂, Y₃, and Y₄ are CH or N.

13. The compound of claim 12, selected from the group consisting of:

| | |
|---|---|
| 63. | 3-(4-cyclopropyl-6-methoxypyrimidin-5-yl)-1-methyl-5-(6-(1-methyl-4- |
| | (trifluoromethyl)-1H-imidazol-2-yl)pyridazin-3-yl)-4,5,6,7-tetrahydro-1H- |
| | pyrazolo[4,3-c]pyridine |
| 64. | 3-(4-cyclopropyl-6-methoxypyrimidin-5-yl)-1-methyl-5-(2-(1-methyl-4- |
| | (trifluoromethyl)-1H-imidazol-2-yl)pyrimidin-5-yl)-4,5,6,7-tetrahydro-1H- |
| | pyrazolo[4,3-c]pyridine |
| 65. | 2-(3-(4-cyclopropyl-6-methoxypyrimidin-5-yl)-5-(5-(1-methyl-4-(trifluoromethyl)- |
| | 1H-imidazol-2-yl)pyridin-2-yl)-4,5,6,7-tetrahydro-1H-pyrazolo[4,3-c]pyridin-1-yl)- |
| | N,N-dimethylethan-1-amine |
| 66. | 3-(4-cyclopropyl-6-methoxypyrimidin-5-yl)-5-(5-(1-methyl-4-(trifluoromethyl)-1H- |
| | imidazol-2-yl)pyridin-2-yl)-1-(1-methylazetidin-3-yl)-4,5,6,7-tetrahydro-1H- |
| | pyrazolo[4,3-c]pyridine |
| 67. | 3-(4-cyclopropyl-6-methoxypyrimidin-5-yl)-5-(5-(1-methyl-4-(trifluoromethyl)-1H- |
| | imidazol-2-yl)pyridin-2-yl)-1-(oxetan-3-yl)-4,5,6,7-tetrahydro-1H-pyrazolo[4,3- |
| | c]pyridine |
| 68. | 3-(4-cyclopropyl-6-methoxypyrimidin-5-yl)-5-(5-(1-methyl-4-(trifluoromethyl)-1H- |
| | imidazol-2-yl)pyridin-2-yl)-1-(pyridin-2-ylmethyl)-4,5,6,7-tetrahydro-1H- |
| | pyrazolo[4,3-c]pyridine |
| 69. | 3-(4-cyclopropyl-6-methoxypyrimidin-5-yl)-5-(5-(1-methyl-4-(trifluoromethyl)-1H- |
| | imidazol-2-yl)pyridin-2-yl)-1-(pyridin-3-ylmethyl)-4,5,6,7-tetrahydro-1H- |
| | pyrazolo[4,3-c]pyridine |
| 70. | 3-(4-cyclopropyl-6-methoxypyrimidin-5-yl)-5-(5-(1-methyl-4-(trifluoromethyl)-1H- |
| | imidazol-2-yl)pyridin-2-yl)-1-(pyrimidin-2-ylmethyl)-4,5,6,7-tetrahydro-1H- |
| | pyrazolo[4,3-c]pyridine |
| 71. | 3-(4-cyclopropyl-6-methoxypyrimidin-5-yl)-5-(5-(1-methyl-4-(trifluoromethyl)-1H- |
| | imidazol-2-yl)pyridin-2-yl)-1-(pyrazin-2-ylmethyl)-4,5,6,7-tetrahydro-1H- |
| | pyrazolo[4,3-c]pyridine |
| 72. | 3-(4-cyclopropyl-6-methoxypyrimidin-5-yl)-5-(5-(1-methyl-4-(trifluoromethyl)-1H- |
| | imidazol-2-yl)pyridin-2-yl)-1-(pyridazin-3-ylmethyl)-4,5,6,7-tetrahydro-1H- |
| | pyrazolo[4,3-c]pyridine |
| 73. | 3-(4-cyclopropyl-6-methoxypyrimidin-5-yl)-1-(4-fluorobenzyl)-5-(5-(1-methyl-4- |
| | (trifluoromethyl)-1H-imidazol-2-yl)pyridin-2-yl)-4,5,6,7-tetrahydro-1H- |
| | pyrazolo[4,3-c]pyridine |
| 74. | 3-(4-cyclopropyl-6-methoxypyrimidin-5-yl)-1-(3-fluorobenzyl)-5-(5-(1-methyl-4- |
| | (trifluoromethyl)-1H-imidazol-2-yl)pyridin-2-yl)-4,5,6,7-tetrahydro-1H- |
| | pyrazolo[4,3-c]pyridine |
| 75. | 3-(4-cyclopropyl-6-methoxypyrimidin-5-yl)-1-(2-fluorobenzyl)-5-(5-(1-methyl-4- |
| | (trifluoromethyl)-1H-imidazol-2-yl)pyridin-2-yl)-4,5,6,7-tetrahydro-1H- |
| | pyrazolo[4,3-c]pyridine |
| 76. | 3-(4-cyclopropyl-6-methoxypyrimidin-5-yl)-1-(4-chlorobenzyl)-5-(5-(1-methyl-4- |
| | (trifluoromethyl)-1H-imidazol-2-yl)pyridin-2-yl)-4,5,6,7-tetrahydro-1H- |
| | pyrazolo[4,3-c]pyridine |
| 77. | 3-(4-cyclopropyl-6-methoxypyrimidin-5-yl)-1-(3-chlorobenzyl)-5-(5-(1-methyl-4- |
| | (trifluoromethyl)-1H-imidazol-2-yl)pyridin-2-yl)-4,5,6,7-tetrahydro-1H- |
| | pyrazolo[4,3-c]pyridine |
| 78. | 3-(4-cyclopropyl-6-methoxypyrimidin-5-yl)-1-(2-chlorobenzyl)-5-(5-(1-methyl-4- |
| | (trifluoromethyl)-1H-imidazol-2-yl)pyridin-2-yl)-4,5,6,7-tetrahydro-1H- |
| | pyrazolo[4,3-c]pyridine |
| 79. | 3-(4-cyclopropyl-6-methoxypyrimidin-5-yl)-1-(4-methoxybenzyl)-5-(5-(1-methyl-4- |
| | (trifluoromethyl)-1H-imidazol-2-yl)pyridin-2-yl)-4,5,6,7-tetrahydro-1H- |
| | pyrazolo[4,3-c]pyridine |
| 80. | 3-(4-cyclopropyl-6-methoxypyrimidin-5-yl)-1-(3-methoxybenzyl)-5-(5-(1-methyl-4- |
| | (trifluoromethyl)-1H-imidazol-2-yl)pyridin-2-yl)-4,5,6,7-tetrahydro-1H- |
| | pyrazolo[4,3-c]pyridine; and |
| 81. | 3-(4-cyclopropyl-6-methoxypyrimidin-5-yl)-1-(2-methoxybenzyl)-5-(5-(1-methyl-4- |
| | (trifluoromethyl)-1H-imidazol-2-yl)pyridin-2-yl)-4,5,6,7-tetrahydro-1H- |
| | pyrazolo[4,3-c]pyridine |

or a pharmaceutically acceptable salt or a stereoisomer thereof.

14. A compound of formula (IJ): 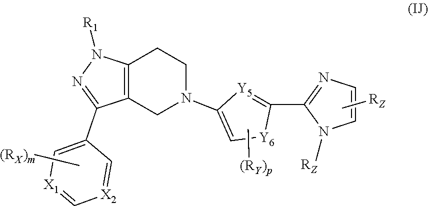
or a pharmaceutically acceptable salt thereof or a stereoisomer thereof, wherein:
each X₁ and X₂ independently is N or C;
each Y₅ and Y₆ independently is N, C or S;
R_{X} at each occurrence independently is alkyl, alkoxy or cycloalkyl;
R_{Y} is halo, alkyl, alkoxy or alkylamino;
R_{Z} at each occurrence is independently selected from hydrogen, halo, alkyl, haloalkyl, alkoxy, cycloalkyl, alkylaminoalkyl, alkoxyalkyl, and unsubstituted or alkyl-substituted heterocycloalkyl;
R₁ is selected from hydrogen, alkyl, cycloalkyl, alkylaminoalkyl, unsubstituted or alkyl-substituted heterocycloalkyl, unsubstituted or substituted arylalkyl, and unsubstituted or substituted heteroarylalkyl, wherein the arylalkyl and heteroarylalkyl substituents are selected from halo, alkyl and alkoxy;
m is 1, 2 or 3; and
p is 0 or 1.

15. The compound of claim 14 which is:
3-(4-cyclopropyl-6-methoxypyrimidin-5-yl)-1-methyl-5-(5-(1-methyl-4-(trifluoromethyl)-1H-imidazol-2-yl)thiophen-3-yl)-4,5,6,7-tetrahydro-1H-pyrazolo[4,3-c]pyridine; or
4-(3-(4-cyclopropyl-6-methoxypyrimidin-5-yl)-1-methyl-1,4,6,7-tetrahydro-5H-pyrazolo[4,3-c]pyridin-5-yl)-2-(1-methyl-4-(trifluoromethyl)-1H-imidazol-2-yl)thiazole;
or a pharmaceutically acceptable salt thereof or a stereoisomer thereof.

16. A pharmaceutical composition comprising a compound of any one of claims 1 to 15, or a pharmaceutically acceptable salt thereof or a stereoisomer thereof, and one or more pharmaceutically acceptable carriers or excipients.

17. The compound of any one of claims 1 to 15, or a pharmaceutically acceptable salt or a stereoisomer thereof, for use as a medicament.

18. The compound of any one of claims 1 to 15, or a pharmaceutically acceptable salt or a stereoisomer thereof, for use in the treatment of cancer.

19. The compound of claim 18, wherein the cancer is a hematological cancer or a lymphatic cancer.

20. The compound of claim 18 or claim 19 wherein the cancer is a DNA damage repair pathway deficient cancer and/or a homologous-recombination deficient cancer.

21. The compound of one of claims 18 to 20, wherein the cancer comprises cancer cells with a mutation in a gene encoding p53.

22. The compound of claim 21, wherein the mutation in the gene encoding p53 is a loss of function mutation.

23. A method of inhibiting USP1 in a subject comprising administering to the subject an effective amount of a compound of any one of claims 1-15 or a pharmaceutically acceptable salt or a stereoisomer thereof.

24. A method of treating a disease or disorder mediated by USP1 in a subject comprising administering to the subject a therapeutically effective amount of a compound of any one of claims 1 to 15 or a pharmaceutically acceptable salt or a stereoisomer thereof.

25. The method of claim 24, wherein the said disease or disorder is cancer.

26. The method of claim 25, wherein the cancer is a hematological cancer or a lymphatic cancer.

27. The method of claim 25 or claim 26, wherein the cancer is a DNA damage repair pathway deficient cancer and/or a homologous-recombination deficient cancer.

28. The method of any one of claims 24-27, wherein the cancer comprises cancer cells with a mutation in a gene encoding p53.

29. The method of claim 28, wherein the mutation in the gene encoding p53 is a loss of function mutation.
